(19) United States
(12) Patent Application Publication
(10) Pub. No.: US 2026/0258296 A1
(43) Pub. Date: Sep. 3, 2026

(54) ORGANIC MULTI-LEVEL QUANTUM SYSTEM AND ITS APPLICATIONS THEREOF

(71) Applicants: THE TRUSTEES OF INDIANA UNIVERSITY, Bloomington, IN (US); University of Notre Dame du Lac, South Bend, IN (US)

(72) Inventors: Bidisha BISWAS, Bloomington, IN (US); Ricardo Javier VAZQUEZ, Bloomington, IN (US); Sarah COX-VAZQUEZ, Bloomington, IN (US); Kingsley BORTEY, Bloomington, IN (US); Aliaksandra LISOUSKAYA, South Bend, IN (US)

(21) Appl. No.: 19/554,322

(22) Filed: Mar. 2, 2026

Related U.S. Application Data
(60) Provisional application No. 63/766,148, filed on Mar. 3, 2025.

(51) Int. Cl. C09K 11/07; C07D 403/10; G01N 21/64; G01N 21/76; G16C 10/00

(52) U.S. Cl. CPC C09K 11/07; C07D 403/10; G01N 21/6456; G01N 21/76; G16C 10/00

(57) ABSTRACT

The present disclosure relates to charge transfer oligomers, such as halogenated or ionic charge transfer oligomers (HCTOs/ICTOs), useful as multi-level quantum systems. Provided are compositions comprising non-ionic and ionic organic molecules with accessible multiple quantum states. In addition, also provided are compositions comprising a HCTO/ICTO, such as a conjugated oligoelectrolyte (COE), which is water soluble, and methods of using an example COE in compositions in aqueous and biological applications.

### RELATED APPLICATIONS

This application claims the benefit of U.S. Provisional Application No. 63/766,148, filed Mar. 3, 2025, the entire disclosure of which is incorporated herein by reference.

### STATEMENT OF GOVERNMENT RIGHTS

This invention was made with government support under R35GM157146 awarded by the National Institute of General Medical Sciences of the National Institutes of Health. The Government has certain rights in the invention.

### TECHNICAL FIELD

The present disclosure relates to charge transfer oligomers, such as halogenated charge transfer oligomers (HCTOs) and ionic charge transfer oligomers (ICTOs), useful as quantum systems. Provided are compositions comprising non-ionic and ionic organic molecules with multiple energetically overlapping and accessible quantum states. Also provided are compositions comprising the charge transfer oligomers (e.g., HCTOs/ICTOs), which could be water soluble, and methods of using an example ICTO in compositions in aqueous and biological applications.

### BACKGROUND

Quantum information processing relies on two key types of quantum systems: qubits and qudits. Qubits operate with two discrete states, forming the basis of most quantum logic operations. On the other hand, qudits extend beyond two level, with three-level systems known as qutrits—a particularly important subclass. Qutrits offer greater information density per unit and improved fault tolerance, making them advantageous for quantum error correction and more efficient quantum algorithms. While qubits remain the standard, qudits could provide enhanced computational capabilities, positioning them as key components in the evolution of quantum technologies.

The common denominator among these quantum systems is their ability to access multiple quantum states, namely, singlet states (Sₙ), doublet states (Dₙ), and triplet states (Tₙ), upon photophysical excitation or operation. The Sₙ state represents a high-probability quantum mechanical transition with a singular orientation, resulting in a relatively short lifetime and less suitable for quantum coherence applications. In contrast, Dₙ and Tₙ states possess multiple spatial orientations that can be simultaneously occupied, providing access to multi-level quantum phenomena. For Dₙ and Tₙ, these spatial projections are highly desirable and associated with electron spins. Still, these low-probable quantum mechanical transitions are directly linked to long-lived spin quantum coherence. Although Sₙ plays a role in multilevel quantum systems, Dₙ and Tₙ are more strongly associated with spin quantum coherence, as the lifetimes of their multiple spin projections are orders of magnitude longer than the single projection of Sn. Despite their significance, minimal scaffolds could access their Dₙ, Tₙ, or a combination thereof upon photophysical excitation or operation. Maintaining long-lived quantum coherence is crucial because it reduces error accumulation during quantum operations, thereby enhancing the overall efficiency and reliability of quantum information processing. Note that multi-component long-lived decays of such multi-level quantum states are associated with quantum-coherence decay (loss), ergo decoherence.

The future success of quantum biosensing depends on developing probes with quantum character capable of extracting information in biological-relevant microenvironments that is often unattainable using traditional probes governed by classical mechanics. Current understanding suggests that qubits can deliver high-resolution optical or electronic readouts (analytical signals) sensitive to subtle electric field effects (EFEs) and magnetic field effects (MFEs), including those often associated with biological events. A rare example of such a quantum bioprobe operable at room temperature is the negatively charged nitrogen-vacancy (NV⁻) center in diamond. This stable point defect hosts an extra electron at the vacancy site, forming a ground-state triplet (Spin=1). Such a ground state can be manipulated in two superimposed spin states, thus constituting an archetypal solid-state qubit. Consequently, NV⁻-based qubits have enabled the detection of DNA duplexes by leveraging nuclear magnetic resonance (NMR) and continuous-wave electron paramagnetic resonance (CW-EPR) principles. However, NV⁻-based qubits typically operate as solid-state sensors, necessitating the immobilization of intact biomolecules onto the diamond surface, which compromises their application in freely diffusive biological systems.

Recently, photogenerated, transient but polarizable spin-correlated radical pairs (SCRP) derived from solution-processable organic chromophores have emerged as promising qubit candidates. Their two entangled spin states form a superimposed quantum state, constituting a prototypical two-level quantum system, or qubit. For context, in SCRP photophysics (see FIG. 1), a subnanosecond photoinduced electron transfer occurs from an electron-rich donor to an electron-deficient acceptor, generating two spatially separated but spin-entangled radicals. Each charge-separated radical ion bears a doublet spin multiplicity (Dₙ−Spin=1/2), forming a radical pair with initial singlet character (¹SCR-P). In some cases, a charge recombination step associated with back electron transfer (bCRS) can regenerate the emissive singlet from ¹SCRP. Through radical-pair intersystem crossing (RP-ISC), this ¹SCRP can convert into a spin-polarized triplet (³SCRP), enabling magnetic polarization of the charge-separated spin state and rendering it CW-EPR detectable and MFE and EFE susceptible. This ³SCRP can then recombine via a back electron transfer mechanism (bCRT), yielding a molecular triplet that could also be CW-EPR active, which relaxes via non-radiative ISC to the ground state. Such radical pair photophysics are often based on naphthalene-diimide (NDI), perylenediimide (PDI), and BODIPY derivatives, due to their intrinsic triplet-mediated photophysics. Another emerging strategy for generating quantum addressable chromophores involves incorporating nitronyl nitroxide radical moieties (Spin=1/2) into the molecular backbone. This design yields synthetically tunable chromophores featuring open-shell exchange-coupled radicals (ECRs). A common feature for SCRPs and ECRs is their ability to access multiple photogenerated spin states, primarily at cryogenic temperatures, where reduced molecular motions help preserve quantum coherence. In these regimes, transient spins can be manipulated and addressed primarily through radical pair exchange coupling (J_{RP}) and dipolar coupling (D) interactions.

Based on principles consistent with Anderson's perturbation framework, the energy levels of singlet and triplet radical pairs reflect their local electronic configurations when both share the same donor-acceptor orbital origin, with J_{RP} being proportional to the energy splitting between these spin states. Therefore, J_{RP} is proportional to the electron exchange constant (K_{HL}) associated with the energy gap between the singlet (S₁) and triplet (T₁) manifolds (J_{RP}~ΔE_{ST}/2 KHL), a relationship that also applies to excitonic systems such as those exhibiting thermally activated delayed fluorescence (TADF) characteristics. The current understanding suggests that minimizing J_{RP} is beneficial for obtaining qubits, as a small J_{RP} facilitates photoinduced spin-manifold mixing, resulting in a larger spin-polarized signal. Matching J_{RP} values to the magnitude of the hyperfine coupling constant (Aₓ) is desirable, as this condition enhances the sensitivity of both EPR spectral features and radical recombination kinetics to EEFs and MFEs. Such effects can occur even under magnetic fields below 1 T, where the magnetic energy scale is approximately four orders of magnitude smaller than room temperature Boltzmann thermal energy (k_{BT}≈25 meV). Despite recent progress in solution-processable quantum probes, achieving biocompatible systems that retain room-temperature spin-polarized properties while remaining structurally stable in aqueous environments for sensing freely diffusing biomolecules remains a significant challenge.

### BRIEF SUMMARY OF THE DISCLOSURE

Disclosed herein are charge transfer oligomers, a class of molecules with widespread applications ranging from biology to energy transduction. The charge transfer oligomer may be a halogenated charge transfer oligomer (HCTO) or an ionic charge transfer oligomer (ICTO). For example, the charge transfer oligomer may be a conjugated oligoelectrolyte (COE).

In one aspect, the disclosure provides a compound of the formula I,

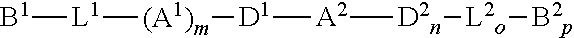

or a salt thereof, wherein
   each of A¹ and A² is independently an electron acceptor;
   each of D¹ and D² is independently an electron donor;
   each of L¹ and L² is independently a π-conjugated linker;
   each of B¹ and B² is independently an aryl substituted with one to four alkyl or alkoxy chains, wherein each of the alkyl or alkoxy chains are substituted with halogens, ionic groups, or water-solubilizing groups;
   m is 0 or 1;
   n is 0 or 1;
   o is 0 or 1; and
   p is 0 or 1.

In some embodiments, the disclosure provides a compound of Formula I, provided that:
(i) m is 0 and n, o, and p are each 1;
(ii) n is 1 and m, o, and p are each 0;
(iii) each of m, n, o, and p are 0; or
(iv) each of m, n, o, and p are 1.

In another aspect, the disclosure provides a composition comprising a compound according to the disclosure, and a liposome.

In another aspect, the disclosure provides a method of delivering a compound to a cell comprising contacting a cell with a composition of the disclosure or a compound of the disclosure.

In another aspect, the disclosure provides a method of imaging a biological sample, the method comprising
(i) contacting a biological sample with a composition of the disclosure or a compound of the disclosure;
(ii) exposing the biological sample to light of a first wavelength; and
(iii) detecting an emission of a second wavelength in the biological sample.

Additional embodiments, features, and advantages of the disclosure will be apparent from the following detailed description and through practice of the disclosure. It will be understood that any of the embodiments described herein can be used in connection with any other embodiments described herein to the extent that the embodiments do not contradict one another.

### BRIEF DESCRIPTION OF THE FIGURES

FIG. 1 is a schematic representation of an energy diagram for COE-CbzBP (ICTO-BPC) illustrating the electronic transitions of a prototypical photogenerated spin-correlated radical pair (SCRP).

FIG. 2 shows the steady-state absorption properties of HCTO-BPC in different organic solvents.

FIG. 3A shows the steady-state fluorescence properties of HCTO-BPC compounds in different organic solvents, and FIG. 3B shows the representative steady-state gated emission properties of HCTO-BPC compounds to determine the singlet and triplet energy levels.

FIG. 4A shows the transient absorption profile of the HCTO-BPC compound in dichloromethane. FIG. 4B shows the representative transient absorption decay profile at 560 nm of the HCTO-BPC compound in dichloromethane (DCM) show long-lived multi-component decay.

FIGS. 5A and 5B show plots illustrating the (FIG. 5A) absorption and (FIG. 5B) emission response of ICTO-BPC (10 μM) in different aqueous solutions.

FIG. 6A shows a plot illustrating gated phosphorescence measurement of ICTO-BPC (10 μM) at 77 K showing energetically overlapping quantum states; and FIGS. 6B-6F show plots illustrating the emission response of ICTO-BPC (10 μM) in (FIG. 6B) MillQ, (FIG. 6C) in 1×PBS, (FIG. 6D) in 0.5 M NaCl, (FIG. 6E) in 1 M NaCl and (FIG. 6F) in 3 M NaCl (λₑₓ=380 nm).

FIGS. 7A-7F show plots illustrating the fluorescence lifetime profiles of ICTO-BPC in (FIG. 7A) MillQ, (FIG. 7B) 1×PBS, (FIG. 7C) 0.5 M NaCl, (FIG. 7D) 1 M NaCl, (FIG. 7E) 2 M NaCl, and (FIG. 7F) 3 M NaCl, respectively, with excitation at 375 nm.

FIG. 8 shows a plot of the emission response of ICTO-BPC (10 μM) in pure water with increasing concentrations of Glycerol (0-60%).

FIGS. 9A-9F show plots illustrating the time-resolved transient absorption spectra of ICTO-BPC under purged and unpurged conditions in (FIG. 9A) MilliQ, (FIG. 9B) 1×PBS, (FIG. 9C) 0.5 M NaCl, (FIG. 9D) 1M NaCl, (FIG. 9E) 2M NaCl, and (FIG. 9F) 3M NaCl.

FIGS. 10A and 10B show plots illustrating the time-resolved decay plots of ICTO-BPC in (FIG. 10A) pure water and 3 M NaCl and (FIG. 10B) in MilliQ and PBS under purged and unpurged conditions. The multi-component long-lived decays are associated with quantum-coherence decay, ergo decoherence. FIGS. 10C-10F show plots illustrating the transient decay kinetics plot of ICTO-BPC in (FIG. 10C) MilliQ, (FIG. 10D) MilliQ (purged), (FIG. 10E) 3M NaCl, and (FIG. 10F) 3M NaCl (purged).

FIGS. 11A-11C show plots illustrating the time-resolved transient absorption spectra of ICTO-BPC under purged and unpurged conditions in different percentages of glycerol (FIG. 11A) 20% gly, (FIG. 11B) 40% gly and (FIG. 11C) 60% gly under unpurged and purged conditions.

FIG. 12 shows a comparison of the time-resolved transient absorption decay of ICTO-BPC in different solutions and environments under unpurged conditions. The multi-component long-lived decays are associated with quantum-coherence decay, ergo decoherence.

FIG. 13 illustrates a DLS analysis of ICTO-BPC intercalated 100 nm extruded liposomes composed of a 3:1 ratio of POPC:POPG in various solvents. Concentrations for COE-CbzBP was 1 mol % when tested with liposomes.

FIGS. 14A and 14B show plots illustrating (FIG. 14A) fluorescence emission and (FIG. 14B) lifetime profiles of the ICTO-BPC intercalated 100 nm extruded liposomes composed of a 3:1 ratio of POPC:POPG. FIG. 14C shows a plot illustrating the absorption spectra of COE-CBz-BP:Lipo. Concentrations for COE-CbzBP was 1 mol % when tested with liposomes.

FIGS. 15A and 15B show plots illustrating time-resolved transient absorption spectra of ICTO-BPC intercalated 100 nm extruded liposomes in (FIG. 15A) MilliQ and (FIG. 15B) PBS and composed of 3:1 ratio of POPC and POPG. FIG. 15C shows a time-resolved decay plot of ICTO-BPC in MilliQ, PBS, and 2% PBS. The ICTO-BPC intercalated in liposomes shows a single component assignable to triplets, which means it loses its doublet state character in these environments.

FIGS. 16A-16C show plots illustrating the absorption spectra of ICTO-BPC (20 μM) in the presence of 0.25 and 0.5 mg/mL of DNA in (FIG. 16A) MilliQ, (FIG. 16B) 1×PBS and (FIG. 16C) 10 mM TRIS.

FIGS. 17A-17C show plots illustrating the emission spectra of ICTO-BPC (20 μM) in the presence of 0.25 and 0.5 mg/mL of DNA in (FIG. 17A) MilliQ, (FIG. 17B) 1×PBS and (FIG. 17C) 10 mM TRIS.

FIGS. 18A-18C show plots illustrating the fluorescence lifetime spectra of ICTO-BPC (20 μM) in the presence of 0.25 and 0.5 mg/mL of DNA in (FIG. 18A) MilliQ, (FIG. 18B) 1×PBS and (FIG. 18C) 10 mM TRIS.

FIGS. 19A-19I show plots illustrating the time-resolved transient absorption spectra and time-resolved decay plots of ICTO-BPC in the absence and presence of DNA. Time-resolved transient absorption spectra of ICTO-BPC in the absence and presence of DNA in (FIGS. 19A and 19B) MilliQ, (FIGS. 19D and 19E) 1×PBS and (FIGS. 19G and 19H) 10 mM Tris. Time-resolved decay plots of ICTO-BPC in the absence and presence of DNA in (FIG. 19C) MilliQ, (FIG. 19F) 1×PBS, and (FIG. 19I) 10 mM Tris. The multi-component long-lived decays are associated with quantum-coherence decay, ergo decoherence.

FIGS. 20A and 20B show plots illustrating the time-resolved transient absorption spectra of ICTO-BPC in the absence and presence of DNA in MilliQ, 1×PBS, and 10 mM Tris at (FIG. 20A) 6 ns and (FIG. 20B) 1 μs time-points. The multi-component long-lived decays are associated with quantum-coherence decay, ergo decoherence.

FIGS. 21A and 21B show plots illustrating (FIG. 21A) absorption and (FIG. 21B) emission of ICTO-BPC with BSA (15 μM) in 1×PBS.

FIG. 22A shows an energy diagram illustrating the electronic transitions of a prototypical photogenerated spin-correlated radical pair (SCRP). FIG. 22B shows the molecular structure of the COE-CbzBP. FIG. 22C shows the HOMO-LUMO spatial distribution of the COE-CbzBP, revealing minimal orbital overlap. FIG. 22D shows the resonance structure of the SCRP in a twisted conformation. FIG. 22E shows the biophysical integration of the COE-CbzBP into DNA, in close proximity with DNA's static electric field (E→). This illustration does not imply groove binding or intercalation interactions. FIG. 22F shows the resonance structure of the zwitterionic form in a planar conformation. FIG. 22G shows the spontaneous intercalation of the COE-CbzBP into lipid bilayers.

FIG. 23A shows a plot illustrating steady state absorption and emission spectra of the COE-CbzBP in pure water. The emission spectra were measured at 77K under oxygen-free conditions with a gated detector, revealing ΔE_{ST}~0.12 eV. FIG. 23B shows a plot illustrating normalized emission spectra of the COE-CbzBP at room temperature in different environments, demonstrating TICT character. All solution measurements were performed with 10-20 M COE-CbzBP and 1% mol in liposomes.

FIGS. 24A-24C show a plots illustrating continuous-wave electron paramagnetic resonance (CW-EPR) spectra of the COE-CbzBP at 1 mM concentration, unless indicated otherwise, under the following conditions: (FIG. 24A) light ON and OFF in pure water, (FIG. 24B) light ON at cryogenic and room temperatures in 60% glycerol, (FIG. 24C) light ON in the presence of the spin-selective trap 4-POBN (40×) in pure water. FIG. 24D shows a proposed mechanism for the formation of the 4-POBN/free radical adduct. FIG. 24E shows a plot illustrating continuous-wave electron paramagnetic resonance (CW-EPR) spectra of the COE-CbzBP at 2 mM in various room-temperature biological environments in 10 mM Tris-HCl solution. The ratio between the COE-CbzBP and DNA was 1:1, where the stock solution of DNA is 5 mg/ml relative to 2 mM of COE-CbzBP.

FIGS. 25A-25C show plots illustrating femtosecond transient absorption spectra of COE-CbzBP in (FIG. 25A) Tris-HCl aqueous solution, (FIG. 25B) with 40× of 4-POBN spin-trap added, and (FIG. 25C) their corresponding kinetic traces. FIGS. 25D-25F show plots illustrating femtosecond transient absorption spectra of COE-CbzBP in Tris-HCl aqueous solutions following (FIG. 25D) association with DNA, (FIG. 25E) intercalation into lipid bilayers, and (FIG. 25F) their respective kinetic traces. All measurements were performed at a concentration of 200 M COE-CbzBP under deoxygenated conditions. UV-Vis spectra before and after TAS confirmed sample stability under all conditions. The concentration of Tris-HCl is 10 mM.

FIGS. 26A and 26B show plots illustrating nanosecond transient absorption spectra snapshot (500 ns) of COE-CbzBP in (FIG. 26A) Tris-HCl aqueous solution and 40× of 4-POBN spin-trap added, with (FIG. 26B) their corresponding kinetic traces. FIGS. 26C and 26D show plots illustrating nanosecond transient absorption spectra snapshot (500 ns) of COE-CbzBP in (FIG. 26C) Tris-HCl aqueous solutions without a permanent magnet (−MF) and with a permanent magnet (+MF), with (FIG. 26D) their corresponding kinetic traces. A permanent magnet providing ~550 mT at its surface was used. The effective magnetic field at the pump-probe interaction region, considering the sample-to-magnet standoff distance, was measured to be ~270 mT using a Gaussmeter. FIGS. 26E and 26F show plots illustrating nanosecond transient absorption spectra snapshot (500 ns) of COE-CbzBP in (FIG. 26E) Tris-HCl aqueous solution, upon association with DNA, and intercalation into lipid bilayers, with (FIG. 26F) their corresponding kinetic traces. All measurements were performed at a concentration of 200 μM COE-CbzBP under deoxygenated conditions. UV-Vis spectra before and after TAS confirmed sample stability under all conditions. The concentration of Tris-HCl is 10 mM.

FIGS. 27A and 27B show energy diagrams of COE-CbzBP electronic transitions when (FIG. 27A) interfaced with DNA, showing SCRP behavior, and (FIG. 27B) intercalated into liposomes, where no SCRP behavior is observed. Rate constants were determined using quantum yield, fluorescence lifetimes, and nanosecond-scale transient absorption data.

FIGS. 28A and 28B show plots illustrating absorption spectra of the COE-CbzBP measured at 10 μM in (FIG. 28A) Ultra-Pure, 1×PBS and 10 mM Tris-HCl and (FIG. 28B) glycerol solutions. Excitation spectra of COE-CbzBP in Ultra-Pure water. FIG. 28C shows a plot illustrating excitation spectra of COE-CbzBP in Ultra-Pure water.

FIG. 29 shows a plot illustrating a representative cyclic voltammetry (CV) of immobilized COE-CbzBP in ACN with 0.5 M TBAPF₆.

FIGS. 30A and 30B show plots illustrating circular dichroism absorption profile of the COE-CbzBP, DNA, and COE-CbzBP/DNA complex. Concentration of COE-CbzBP is 14 uM.

FIG. 31A shows a plot illustrating aged CW-EPR spectra of the COE-CbzBP:DNA (1:1) complex recorded on three different days. Thirty scans were carried out for these measurements, for a total of ~45 minutes of constant irradiation. FIG. 31B shows a plot illustrating CW-EPR spectra of the COE-CbzBP:DNA (1:1) complex recorded over successive scans in aged solutions.

FIG. 32 shows a plot illustrating CW-EPR spectrum of 1 mM COE-CbzBP in 0-60% glycerol solutions irradiated with UV-VIS light in situ at room temperature.

FIGS. 33A-33E show plots illustrating UV-Vis measurements for the COE-CbzBP in different environments before and after transient absorption spectroscopy characterization. For the experiments, the concentration of the COE-CbzBP is 200 uM and 1% mol in liposome, POBN is 40×.

FIG. 34 shows plots illustrating spectroelectrochemistry of the COE-CbzBP precursor, product (5), in Acetonitrile.

FIG. 35 shows a plot illustrating absorption spectra of COE-CbzBP (200 uM) upon DNA association or liposome intercalation 5 ns after photoexcitation with 370 nm, characterized with the fs TAS.

FIG. 36 shows plots illustrating femtosecond transient absorption spectroscopy profile of the COE-CbzBP (200 uM) in glycerol.

FIGS. 37A-37C show plots illustrating nanosecond transient absorption spectra of COE-CbzBP in (FIG. 37A) Tris-HCl aqueous solution, (FIG. 37B) with 40× of 4-POBN spin-trap added, and (FIG. 37C) their corresponding kinetic traces. FIGS. 37D-37F show plots illustrating nanosecond transient absorption spectra of COE-CbzBP in Tris-HCl aqueous solutions following (FIG. 37D) association with DNA, (FIG. 37E) intercalation into lipid bilayers, and (FIG. 37F) their respective kinetic traces. All measurements were performed at a concentration of 200 M COE-CbzBP under deoxygenated conditions. UV-Vis spectra before and after TAS confirmed sample stability under all conditions. The concentration of Tris-HCl is 10 mM. These measurements were carried out under magnetic steering conditions. These experiments were under stirring conditions.

FIGS. 38A and 38B show plots illustrating multi-exponential decay fit for the ESA at 530 nm of COE-CbzBP in Tris (FIG. 38A) and COE-CbzBP:DNA in Tris (FIG. 38B), both at 200 uM concentrations.

FIG. 39 shows plots illustrating fluorescence lifetime spectra of COE-CbzBP in different biological environments and glycerol-containing solutions with 10 mM Tris as a buffer. The concentration of COE-CbzBP in only Tris and 40% glycerol was 10 uM, for DNA it was 20 uM, and for liposomes was 1 mol %.

FIGS. 40A and 40B shows plots illustrating nanosecond transient absorption spectroscopy of (FIG. 40A) COE-CbzBP:DNAtitrations in Tris and (FIG. 40B) their corresponding kinetic traces. The concentration of COE-CbzBP was 200 uM.

FIG. 41 shows a plot illustrating normalized nanosecond transient absorption spectra snapshot (500 ns) of COE-CbzBP in Tris-HCl aqueous solutions, upon association with DNA, and intercalation into lipid bilayers. All measurements were performed at a concentration of 200 M COE-CbzBP under deoxygenated conditions.

FIG. 42 shows a plot illustrating nanosecond transient absorption spectroscopy of COE-CbzBP:DNA at ambient and purged conditions. The concentration of COE-CbzBP was 200 uM.

FIG. 43 shows a plot illustrating mono-exponential decay fit for the ESA at 513 nm of COE-CbzBP in liposomes. The concentration of COE-CbzBP in liposome was 1 mol %.

FIG. 44A shows plots illustrating representative nanosecond transient broadband spectroscopy profiles of the COE-CbzBP (200 uM) in glycerol. FIG. 44B shows a plot illustrating multi-exponential decay fit for the ESA at 530 nm of COE-CbzBP in glycerol.

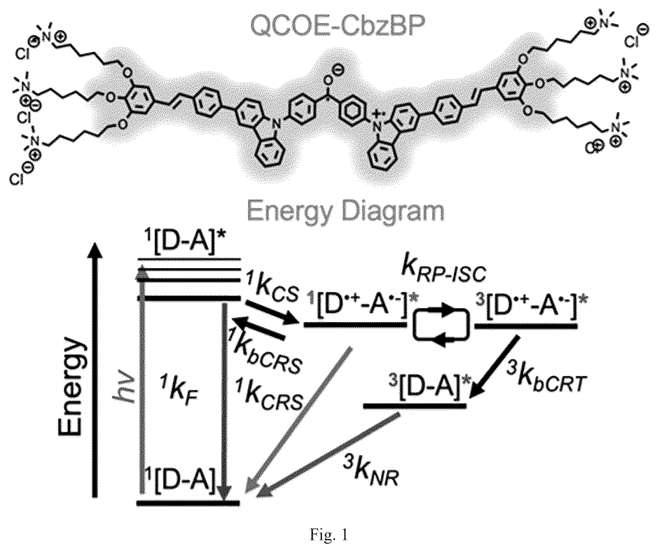

### DETAILED DESCRIPTION OF THE DISCLOSURE

The present disclosure highlights charge transfer oligomers, such as HCTOs/ICTOs, useful as quantum systems. Thus, described herein is a composition of a charge transfer oligomer (e.g., HCTO/ICTO) and methods of accessing and using the compositions described herein. The charge transfer oligomer may be a halogenated charge transfer oligomer (HCTO) or an ionic charge transfer oligomer (ICTO), such as a conjugated oligoelectrolyte (COE).

The compounds of the present disclosure can include a carbazole, a benzophenone, or a combination thereof. For example, the compound may include both a carbazole and a benzophenone.

The compounds of the present disclosure can exhibit magnetic-field sensitive charge-separated states consistent with SCRP character at room temperature. The compounds of the present disclosure can be useful as water-soluble molecular probes, which exhibit electronic and electrochemical properties for SCRP formation.

The developed charge transfer oligomers (e.g., HCTOs/ICTOs) were designed to minimize excited state energy levels (e.g., Sₙ, Dₙ, and Tₙ states), so that the energy penalty is minimal for effective communication between these quantum states, including in aqueous medium and aqueous solutions of various dielectric strengths.

The compounds of the present disclosure possess a spin polarization that can be perturbed by a static electric field. For example, the spin polarization of an exemplary compound of the present disclosure referred to as COE-CbzBP is perturbed by the static electric field of freely diffusing DNA biomolecules, but not by the static electric field of lipid bilayers, as revealed by CW-EPR and transient absorption spectroscopy (TAS).

### Definitions

The following definitions are meant to clarify, but not limit, the terms defined. If a particular term used herein is not specifically defined, such term should not be considered indefinite. Rather, terms are used within their accepted meanings.

As used herein and in the appended clauses, the singular forms “a,” “an,” and “the” include plural referents unless the context clearly dictates otherwise. It is further noted that the clauses may be drafted to exclude any optional element. As such, this statement is intended to serve as antecedent basis for use of such exclusive terminology as “solely,” “only” and the like in connection with the recitation of clause elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

To describe the quantum states associated with quantum coherence, excited state absorption (ESA), transient species, and quantum states are used interchangeably.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Chemical nomenclature for compounds described herein has generally been derived using the commercially-available ACD/Name 2014 (ACD/Labs) or ChemBioDraw Ultra 13.0 (Perkin Elmer).

As used herein, the terms “alkyl” and “alkylene” include a chain of carbon atoms, which is optionally branched and contains from 1 to 20 carbon atoms. The term “alkyl” refers to a straight- or branched-chain monovalent hydrocarbon group. The term “alkylene” refers to a straight- or branched-chain divalent hydrocarbon group. In some embodiments, it can be advantageous to limit the number of atoms in an “alkyl” or “alkylene” to a specific range of atoms, such as C₁-C₂₀ alkyl or C₁-C₂₀ alkylene, C₁-C₁₂ alkyl or C₁-C₁₂ alkylene, or C₁-C₆ alkyl or C₁-C₆ alkylene. Examples of alkyl groups include methyl (Me), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples. Examples of alkylene groups include methylene (—CH₂—), ethylene ((—CH₂—)₂), n-propylene ((—CH₂—)₃), iso-propylene ((—C(H)(CH₃)CH₂—)), n-butylene ((—CH₂—)₄), and the like. It will be appreciated that an alkyl or alkylene group can be unsubstituted or substituted as described herein.

An alkyl or alkylene group can be substituted with any of the substituents in the various embodiments described herein, including one or more of such substituents. It will be understood that “alkyl” or “alkylene” may be combined with other groups, such as those provided above, to form a functionalized alkyl. By way of example, the combination of an “alkyl” group, as described herein, with a “carboxylate” group may be referred to as an “alkylene-carboxylate” group as illustratively represented by the structure:

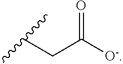

As used herein, the term “alkenyl” includes a chain of carbon atoms, which is optionally branched, and contains from 2 to 20 carbon atoms, and also includes at least one carbon-carbon double bond (i.e., C═C). It will be understood that in certain embodiments, alkenyl may be advantageously of limited length, including C₂-C₁₂, C₂-C₉, C₂-C₈, C₂-C₇, C₂-C₆, and C₂-C₄. Illustratively, such particularly limited length alkenyl groups, including C₂-C₈, C₂-C₇, C₂-C₆, and C₂-C₄ may be referred to as lower alkenyl. Alkenyl may be unsubstituted, or substituted as described for alkyl or as described in the various embodiments provided herein. Illustrative alkenyl groups include, but are not limited to, ethenyl, 1-propenyl, 2-propenyl, 1-, 2-, or 3-butenyl, and the like.

As used herein, the term “aryl” refers to an all-carbon monocyclic or fused-ring polycyclic groups of 6 to 14 carbon atoms having a completely conjugated pi-electron system. It will be understood that in certain embodiments, aryl may be advantageously of limited size such as C₆-C₁₀ aryl. Illustrative aryl groups include, but are not limited to, phenyl, naphthylenyl and anthracenyl. The aryl group may be unsubstituted, or substituted as described for alkyl or as described in the various embodiments provided herein.

As used herein, the term “heteroaryl” refers to a monocyclic or fused ring group of 5 to 12-ring atoms containing one, two, three or four-ring heteroatoms selected from nitrogen, oxygen and sulfur, the remaining ring atoms being carbon atoms, and also having a completely conjugated pi-electron system. It will be understood that in certain embodiments, heteroaryl may be advantageous of a limited size, such as 3- to 7-membered heteroaryl, 5- to 7-membered heteroaryl, and the like. Heteroaryl may be unsubstituted, or substituted as described for alkyl or as described in the various embodiments provided herein. Illustrative heteroaryl groups include heteroaryl groups, include but are not limited to pyrrolyl, furanyl, thiophenyl, imidazolyl, oxazolyl, thiazolyl, pyrazolyl, pyridinyl, pyrimidinyl, quinolinyl, isoquinolinyl, and the like. Illustrative examples of heteroaryl groups shown in graphical representations, include the following entities, in the form of properly bonded moieties:

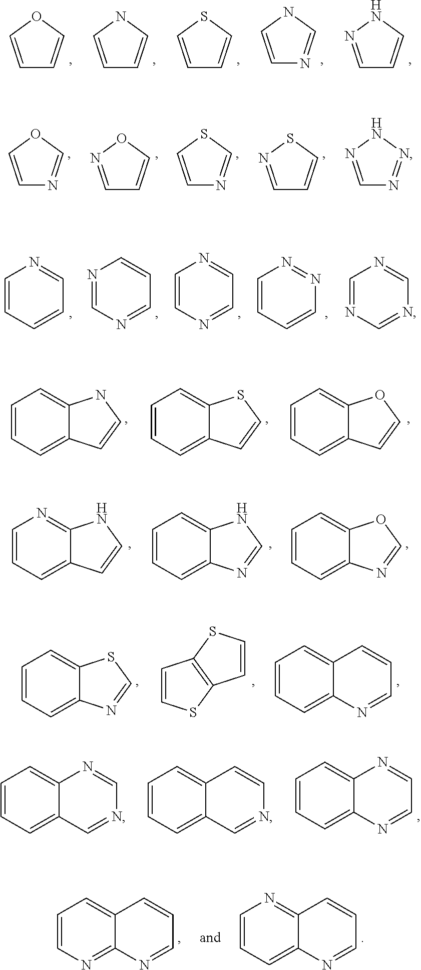

Moreover, the terms “heteroaryl” and “heteroaryl” as used throughout the specification, examples, and claims is intended to include both unsubstituted and substituted heteroaryl and heteroaryl groups, the latter of which refers to heteroaryl and heteroaryl moieties having substituents replacing a hydrogen. In an exemplary embodiment, a heteroaryl or bicyclic heteroaryl includes an oxindole, depicted by the structure

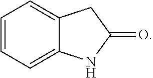

As used herein, “halo” or “halogen” refers to fluorine, chlorine, bromine, or iodine.

As used herein, “cyano” refers to a —CN group.

As used herein, “hydroxy” or “hydroxyl” refers to an —OH group.

As used herein, “carboxylate” refers to a salt or ester of a carboxylic acid represented by the formula —COO⁻.

The term “carboxy,” as used herein, refers to a group represented by the formula CO₂H.

The term “acyl” is art-recognized and refers to a group represented by the general formula alkylC(O)—.

The term “alkoxy” refers to an alkyl group having an oxygen attached thereto. Representative alkoxy groups include methoxy, ethoxy, propoxy, tert-butoxy and the like.

The term “oxo” represents a carbonyl oxygen.

The term “carbonyl” is art-recognized and refers to a group —C(O)— where a double bond exists between the carbon and oxygen.

As used herein, “bond” refers to a covalent bond.

The term “C_{x-y}” or “Cₓ-C_{y},” when used in conjunction with a chemical moiety, such as, acyl, acyloxy, alkyl, alkenyl, alkynyl, or alkoxy is meant to include groups that contain from x to y carbons in the chain. C₀ alkyl indicates a hydrogen where the group is in a terminal position, a bond if internal. A C₁₋₆ alkyl group, for example, contains from one to six carbon atoms in the chain.

As used herein, the suffix “-yl” refers to substituent groups where a radical that is bonded to a radical of an atom on molecule to form a covalent bond. For example, —CH₃ represents a methyl radical that is bonded to a radical atom so that a covalent bond is formed to form a methyl group bonded to the remaining portion of the molecule.

As used herein, the suffix “-ylene” refers to a diradical group that is bonded to two substituents to form two covalent bonds. For example, —CH₂— represents a methylene diradical that is bonded to a radical atom on each side of the carbon so that two covalent bonds are formed to the remaining portions of the molecule. It should be understood that functional groups and substituents described herein with the “-yl” suffix can be readily envisaged to the corresponding “-ylene” suffix to bond to two separate substituents, for example alkyl and alkenyl (forming one bond) would become alkylene and alkenylene, respectively, when forming two bonds.

The term “substituted” means that the specified group or moiety bears one or more substituents. The term “unsubstituted” means that the specified group bears no substituents. Where the term “substituted” is used to describe a structural system, the substitution is meant to occur at any valency-allowed position on the system. In some embodiments, “substituted” means that the specified group or moiety bears one, two, or three substituents. In other embodiments, “substituted” means that the specified group or moiety bears one or two substituents. In still other embodiments, “substituted” means the specified group or moiety bears one substituent. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaryl moiety. It will be understood by those skilled in the art that the moieties substituted on the hydrocarbon chain can themselves be substituted, if appropriate.

As used herein, “optional” or “optionally” means that the subsequently described event or circumstance may but need not occur, and that the description includes instances where the event or circumstance occurs and instances in which it does not. For example, “wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl is independently optionally substituted by C₁-C₆ alkyl” means that an alkyl may be but need not be present on any of the C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl by replacement of a hydrogen atom for each alkyl group, and the description includes situations where the C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl is substituted with an alkyl group and situations where the C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or mono- or bicyclic heteroaryl is not substituted with the alkyl group.

As used herein, “ion” refers to a charged atom or compound. As used herein, “cation” refers to a positively charged ion and “anion” refers to a negatively charged ion. Representative examples of cations include, but are not limited to, sodium, calcium, potassium, and ammonium. Representative examples of anions include, but are not limited to, halide, acetate, mesylate, tosylate, triflate, and bisulfate. Moreover, the term “ionic” may be used to describe a functional group, a structural moiety, or a compound having one or more charged atoms.

The term “salt” is art recognized and refers to a compound including an ion and a counter ion bound by ionic bonds. It will be understood that the chemical entities described herein can exist as a salt of an ion and a counter ion. Salts are typically formed when an acid or a sufficiently acidic group reacts with a base or a sufficiently basic group, resulting in the formation of a salt and water through a chemical reaction called neutralization. Examples of salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates.

The term “counter ion” is art-recognized and refers to an oppositely charged ion that accompanies an ionic species (i.e., “ion”) in order to maintain electric neutrality. A counter ion may represent an anion (negatively charged ion) or a cation (positively charged ion).

As used herein and in connection with chemical structures depicting the various embodiments described herein, “*”, “**”, and “ [symbol] ”, each represent a point of covalent attachment of the chemical group or chemical structure in which the identifier is shown to an adjacent chemical group or chemical structure. For example, in a hypothetical chemical structure A-B, where A and B are joined by a covalent bond, in some embodiments, the portion of A-B defined by the group or chemical structure A can be represented by “A-*”, “A-**”, or

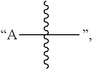

where each of “-*”, “-**”, and

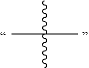

represents a bond to A and the point of covalent bond attachment to B. Alternatively, in some embodiments, the portion of A-B defined by the group or chemical structure B can be represented by “*—B”, “**—B”, or

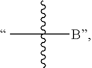

where each of “-*”, “-**” and

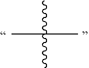

represents a bond to B and the point of covalent bond attachment to A.

As used herein, “independently” means that the subsequently described event or circumstance is to be read on its own relative to other similar events or circumstances. For example, in a circumstance where several equivalent hydrogen groups are optionally substituted by another group described in the circumstance, the use of “independently optionally” means that each instance of a hydrogen atom on the group may be substituted by another group, where the groups replacing each of the hydrogen atoms may be the same or different. Or for example, where multiple groups exist all of which can be selected from a set of possibilities, the use of “independently” means that each of the groups can be selected from the set of possibilities separate from any other group, and the groups selected in the circumstance may be the same or different.

As used herein, the phrase “electron acceptor” refers to a moiety that can accept electron density from a system, and the phrase “electron donor” refers to a moiety that donates electron density into a system. It is to be understood that electron donor and acceptor capabilities are relative; that is, a molecule which can lose an electron under certain experimental conditions will be able to accept an electron under different experimental conditions.

As used herein, an “electron-withdrawing group (EWG)” refers to a group that draws electron density toward itself from neighboring atoms or conjugated systems. Representative examples of EWGs include, but are not limited to, —NO₂, —CN, —CF₃, sulfonic acid (—SO₃H), a halogen, a carbonyl-containing group (e.g., an aldehyde (—CHO), a ketone (—C═O), a carboxylic acid (—COOH), or an ester (—COOalkyl).

As used herein, an “electron-donating group” refers to Representative examples of EWGs include, but are not limited to, hydroxy (—OH), an amine (primary (—NH₂), secondary (—NH—), or tertiary), an ether (—O—), an amide (—NHC(O)alkyl), or an alkyl.

As used herein, a “lipid vesicle” refers to a structure organized in lipid bilayers with an internal (e.g., aqueous) compartment. As used herein, a “liposome” refers to a spherical-shaped vesicle composed of one or more lipid bilayers, which closely resembles the structure of cell membranes. As used herein, a “lipid nanoparticle” or “LNP” refers to spherical vesicles made of ionizable lipids and that are taken up by cells via, endocytosis.

As used herein, the terms “water solubilizing group”, “water soluble group” and “WSG” are used interchangeably and refer to a group or substituent that is well solvated in aqueous environments e.g., under physiological conditions, and that imparts improved water solubility upon the molecules to which it is attached. In some embodiments, a WSG increases the solubility of the compound in a predominantly aqueous solution, as compared to a control compound that lacks the WSG. The water solubilizing groups may be any convenient hydrophilic group that is well solvated in aqueous environments. Illustrative WSG include, but are not limited to carboxylates, ammonium ions (e.g., quaternary amines or quaternary ammonium ions), phosphates, sulfates, choline, and nitrates.

As used herein, a component that is defined as “π-conjugated”, such as a “π-conjugated linker,” is a system of connected p-orbitals with delocalized electrons in a chemical moiety with alternating single and multiple bonds. A π-conjugated linker/system is a chemical moiety that has a region of overlapping p-orbitals, bridging the interjacent single bonds to allow a delocalization of pi electrons across all the adjacent aligned p-orbitals. The pi electrons do not belong to a single bond or atom, but rather to a group of atoms. Illustrative n-conjugated linker groups include, but are not limited to, 1,3-butadiene, beta-carotene, allylic carbocations, thiophene, furan, pyridine, porphyrins, 1,3,5-hexatriene, benzene, vinyl benzene, and other heteroaryl groups as defined above. Additional illustrative 7-conjugated systems include, but are not limited to, benzophenone, naphthalenedione, anthracenedione, benzoquinone, benzonitrile, terephthalonitrile, carbazole, phenoxazine, diphenylamine, and triphenylamine.

The term “biological sample,” as used herein, includes, without limitation, cell cultures or extracts thereof; biopsied material obtained from a mammal or extracts thereof; and blood, saliva, urine, feces, semen, tears, or other body fluids or extracts thereof.

### ILLUSTRATIVE EMBODIMENTS

In one aspect, the disclosure relates to a compound having a 71-conjugated backbone flanked by halogenated or ionic pendant side chains, or a salt thereof. In some embodiments, the compounds of the present disclosure have a partial break in 71-conjugation, such as between an electron donor (e.g., a carbazole) and an electron acceptor (e.g., a benzophenone). In some embodiments the partial break is located at a nitrogen-carbon junction.

In one aspect, the disclosure relates to a compound having the formula (I):

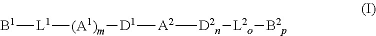

or a salt thereof, wherein
   each of A¹ and A² is independently an electron acceptor;
   each of D¹ and D² is independently an electron donor;
   each of L¹ and L² is independently a π-conjugated linker;
   each of B¹ and B² is independently an aryl substituted with one to four alkyl or alkoxy chains wherein the alkyl or alkoxy chains are substituted with halogens, ionic groups, or water-solubilizing group
   m is 0 or 1;
   n is 0 or 1;
   o is 0 or 1; and
   p is 0 or 1.

In some embodiments, m, n, o, and p are selected from:
(i) m is 0 and n, o, and p are each 1;
(ii) n is 1 and m, o, and p are each 0;
(iii) each of m, n, o, and p are 0; and
(iv) each of m, n, o, and p are 1.

In some embodiments, the compound is of formula

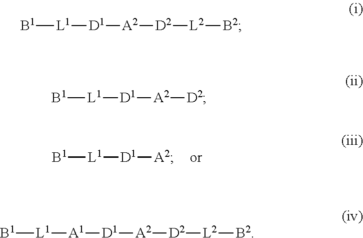

In some embodiments, each A (e.g., A¹ and/or A²) is independently an electron acceptor. In some embodiments, each A is a π-conjugated system comprising an electron-withdrawing group, such as —NO₂, —CN, —CF₃, sulfonic acid (—SO₃H), a halogen, a carbonyl-containing group (e.g., an aldehyde (—CHO), a ketone (—C═O), a carboxylic acid (—COOH), or an ester (—COOalkyl).

In some embodiments, each A (e.g., A¹ and/or A²) is independently of formula (II-A):

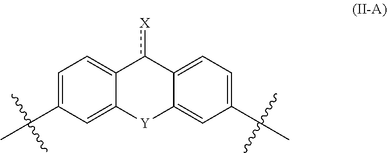

or a salt thereof, wherein
   X is O or CR¹R².
   Y is absent, a bond, C(O), or CR¹R²;
each R¹ is independently CN, C(O)OH, or C(O)—C₁-C₆ alkyl; and
each R² is independently CN, C(O)OH, or C(O)—C₁-C₆ alkyl.

In some embodiments, A (e.g., A¹ and/or A²) is of formula (II-A), or a salt thereof, wherein
X is O or CR¹R².
Y is absent, a bond, C(O), or CR¹R²;
each R¹ is independently CN, C(O)OH, or C(O)O—C₁-C₆ alkyl; and
each R² is independently CN, C(O)OH, or C(O)O—C₁-C₆ alkyl.

In some embodiments, Y is absent, a bond, C(O), or CR¹R². In some embodiments, Y is absent. For example, when Y is absent, A is of the formula

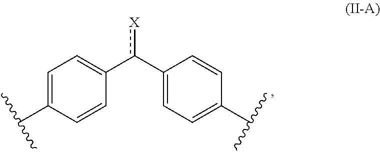

or a salt thereof. In some embodiments, Y is a bond. In some embodiments, Y is C(O). In some embodiments, Y is CR¹R².

In some embodiments, each R¹ is independently CN, C(O)OH, or C(O)—C₁-C₆ alkyl. In some embodiments, each R¹ is independently CN, C(O)OH, or C(O)O—C₁-C₆ alkyl. In some embodiments, one R¹ is CN. In some embodiments, two R¹ are CN. In some embodiments, one R¹ is C(O)OH. In some embodiments, two R¹ are C(O)OH. In some embodiments, one R¹ is C(O)—C₁-C₆ alkyl. In some embodiments, one R¹ is C(O)O—C₁-C₆ alkyl. In some embodiments, two R¹ are C(O)—C₁-C₆ alkyl. In some embodiments, two R¹ are C(O)O—C₁-C₆ alkyl. In some embodiments, one R¹ is C(O)OCH₂CH₃. In some embodiments, two R¹ are C(O)OCH₂CH₃. In some embodiments, one R¹ is CN and one R¹ is C(O)OH. In some embodiments, one R¹ is CN and one R¹ is C(O)OCH₂CH₃. In some embodiments, one R¹ is C(O)OH, and one R¹ is C(O)OCH₂CH₃.

In some embodiments, each A (e.g., A¹ and/or A²) is independently of formula (II-B):

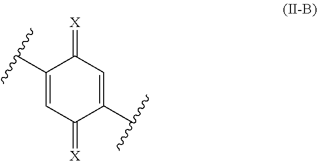

or a salt thereof, wherein
   each X is independently O or CR¹R²;
   each R¹ is independently CN, C(O)OH, or C(O)—C₁-C₆ alkyl; and
   each R² is independently CN, C(O)OH, or C(O)—C₁-C₆ alkyl.

In some embodiments, A (e.g., A¹ and/or A²) is of formula (II-B), or a salt thereof, wherein
each X is independently O or CR¹R²;
each R¹ is independently CN, C(O)OH, or C(O)O—C₁-C₆ alkyl; and
each R² is independently CN, C(O)OH, or C(O)O—C₁-C₆ alkyl.

In some embodiments, each A (e.g., A¹ and/or A²) is independently of formula (II-C):

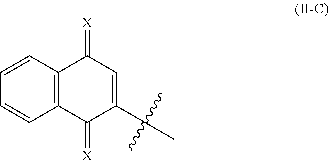

or a salt thereof, wherein
   each X is independently O or CR¹R²;
   each R¹ is independently CN, C(O)OH, or C(O)—C₁-C₆ alkyl; and
   each R² is independently CN, C(O)OH, or C(O)—C₁-C₆ alkyl.

In some embodiments, A (e.g., A¹ and/or A²) is of formula (II-C), or a salt thereof, wherein
each X is independently O or CR¹R²;
each R¹ is independently CN, C(O)OH, or C(O)O—C₁-C₆ alkyl; and
each R² is independently CN, C(O)OH, or C(O)O—C₁-C₆ alkyl.

In some embodiments, X is O or CR¹R². In some embodiments, X is O. In some embodiments, X is CR¹R², for example C(CN)C(O)OH or C(CN)CO(O)alkyl (e.g., C(CN)C(O)OEt).

In some embodiments, each R¹ is independently CN, carboxylate (e.g., C(O)OH), or ester (e.g., C(O)O—C₁-C₆ alkyl). In some embodiments, one R¹ is CN. In some embodiments, two R¹ are CN. In some embodiments, one R¹ is C(O)OH. In some embodiments, two R¹ are C(O)OH. In some embodiments, one R¹ is C(O)—C₁-C₆ alkyl. In some embodiments, one R¹ is C(O)O—C₁-C₆ alkyl. In some embodiments, two R¹ are C(O)—C₁-C₆ alkyl. In some embodiments, two R¹ are C(O)O—C₁-C₆ alkyl. In some embodiments, one R¹ is C(O)OCH₂CH₃. In some embodiments, two R¹ are C(O)OCH₂CH₃. In some embodiments, one R¹ is CN and one R¹ is C(O)OH. In some embodiments, one R¹ is CN and one R¹ is C(O)OCH₂CH₃. In some embodiments, one R¹ is C(O)OH, and one R¹ is C(O)OCH₂CH₃.

In some embodiments, each A (e.g., A¹ and/or A²) is independently of formula (II-D):

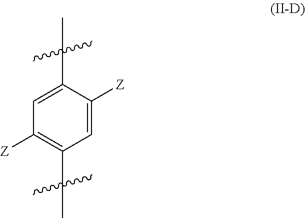

or a salt thereof, wherein
   each Z is independently CN, OH, or OCH₃.

In some embodiments, Z is CN. In some embodiments, Z is OCH₃. In some embodiments, one Z is CN. In some embodiments, one Z is OCH₃. In some embodiments, two Z are CN. In some embodiments, two Z are OCH₃. In some embodiments, one Z is CN and one Z is OCH₃.

In some embodiments, each A (e.g., A¹ and/or A²) is independently of formula (II-E):

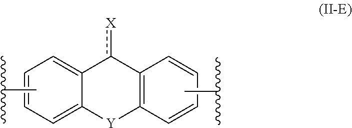

or a salt thereof, wherein
   X is O or CR¹R².
   Y is absent, a bond, O, S, NH, Se, Te, C(O), or CR¹R²;
   each R¹ is independently H, CN, C(O)OH, or C(O)—C₁-C₆ alkyl; and
   each R² is independently H, CN, C(O)OH, or C(O)—C₁-C₆ alkyl.

In some embodiments, X is O or CR¹R². In some embodiments, X is O. In some embodiments, X is CR¹R², for example C(CN)C(O)OH or C(CN)CO(O)alkyl (e.g., C(CN)C(O)OEt).

In some embodiments, Y is absent, a bond, O, S, NH, Se, Te, C(O), or CR¹R². In some embodiments, Y is absent, a bond, C(O), or CR¹R². In some embodiments, Y is absent. For example, when Y is absent, A is of the formula

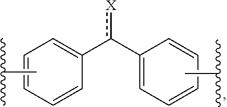

or a salt thereof. In some embodiments, Y is a bond. In some embodiments, Y is O. In some embodiments, Y is S. In some embodiments, Y is NH. In some embodiments, Y is Se. In some embodiments, Y is Te. In some embodiments, Y is C(O). In some embodiments, Y is CR¹R².

In some embodiments, each R¹ is independently CN, C(O)OH, or C(O)O—C₁-C₆ alkyl. In some embodiments, one R¹ is CN. In some embodiments, two R¹ are CN. In some embodiments, one R¹ is C(O)OH. In some embodiments, two R¹ are C(O)OH. In some embodiments, one R¹ is C(O)O—C₁-C₆ alkyl. In some embodiments, two R¹ are C(O)O—C₁-C₆ alkyl. In some embodiments, one R¹ is C(O)OCH₂CH₃. In some embodiments, two R¹ are C(O)OCH₂CH₃. In some embodiments, one R¹ is CN and one R¹ is C(O)OH. In some embodiments, one R¹ is CN and one R¹ is C(O)OCH₂CH₃. In some embodiments, one R¹ is C(O)OH, and one R¹ is C(O)OCH₂CH₃.

In some embodiments, each A (e.g., A¹ and/or A²) is independently a C₆-C₁₄ aryl ring system substituted with or comprising one or more electron-withdrawing group, such as —NO₂, —CN, —CF₃, sulfonic acid (—SO₃H), a halogen, a carbonyl-containing group (e.g., an aldehyde (—CHO), a ketone (—C═O), a carboxylic acid (—COOH), or an ester (—COOalkyl).

In some embodiments, each A (e.g., A¹ and/or A²) is independently a C₆-C₁₄ aryl ring system optionally substituted with halogen, oxo, alkyene, cyano, acyl, alkoxy, and carboxyl. In some embodiments, A is a C₆ aryl ring system optionally substituted with oxo, alkyene, cyano, acyl, alkoxy, and carboxyl. In some embodiments, A is a C₁₀ aryl ring system optionally substituted with oxo, alkyene, cyano, acyl, alkoxy, or carboxyl. In some embodiments, A is a C₁₄ aryl ring system optionally substituted with oxo, alkyene, cyano, acyl, alkoxy, or carboxyl. In some embodiments, A is a C₁₃ aryl ring system optionally substituted with oxo, alkyene, cyano, acyl, alkoxy, or carboxyl.

In some embodiments, each A (e.g., A¹ and/or A²) is independently a benzophenone. For example A may be of the formula

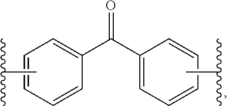

such as

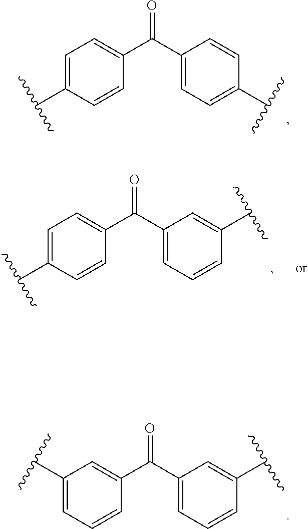 [SMILES: *c1ccc(C(=O)c2ccc(*)cc2)cc1 ; *c1ccc(C(=O)c2cccc(*)c2)cc1 ; *c1cccc(C(=O)c2cccc(*)c2)c1]

In some embodiments, each A (e.g., A¹ and/or A²) is independently a monovalent radical or a bivalent radical. For example, A¹, when present, is a bivalent radical, which forms a covalent bond with L¹ and a covalent bond with D¹. In some embodiments, A² is a monovalent radical, which forms a covalent bond with D¹. In some embodiments, A² is a bivalent radical, which forms a covalent bond with D¹ and a covalent bond with (i) D² when n is 1, (ii) L² when n is 0 and o is 1, or (iii) B² when each of n and o is 0, and p is 1.

In some embodiments, each A (e.g., A¹ and/or A²) is independently selected from Table A.

TABLE A
Electron Acceptor (A) Group Structures

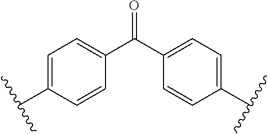 [SMILES: *c1ccc(C(=O)c2ccc(*)cc2)cc1]

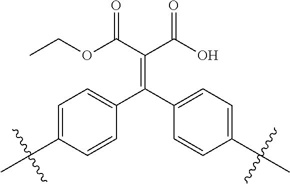 [SMILES: *c1ccc(C(=C(C(=O)O)C(=O)OCC)c2ccc(*)cc2)cc1]

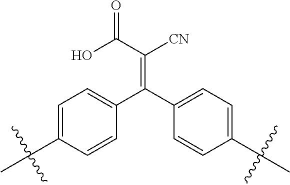 [SMILES: *c1ccc(C(=C(C#N)C(=O)O)c2ccc(*)cc2)cc1]

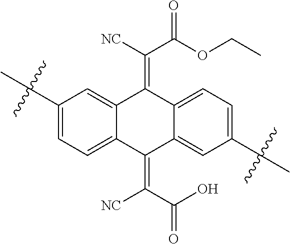 [SMILES: *c1ccc2/c(=C(/C#N)C(=O)OCC)c3cc(*)ccc3/c(=C(\C#N)C(=O)O)c2c1]

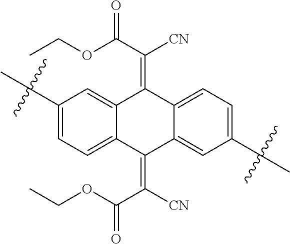 [SMILES: *c1ccc2/c(=C(/C#N)C(=O)OCC)c3cc(*)ccc3/c(=C(\C#N)C(=O)OCC)c2c1]

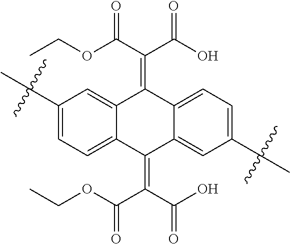 [SMILES: *c1ccc2/c(=C(/C(=O)O)C(=O)OCC)c3cc(*)ccc3/c(=C(\C(=O)O)C(=O)OCC)c2c1]

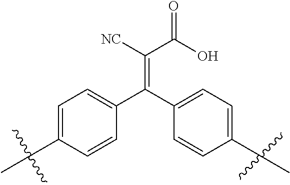 [SMILES: *c1ccc(C(=C(C#N)C(=O)O)c2ccc(*)cc2)cc1]

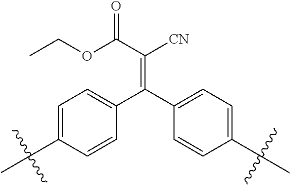 [SMILES: *c1ccc(C(=C(C#N)C(=O)OCC)c2ccc(*)cc2)cc1]

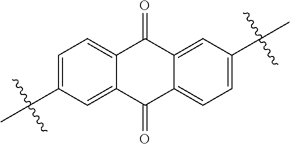 [SMILES: *c1ccc2c(c1)C(=O)c1ccc(*)cc1C2=O]

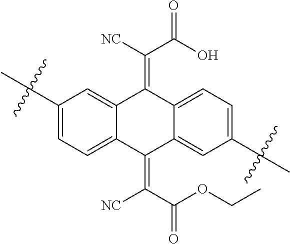 [SMILES: *c1ccc2/c(=C(/C#N)C(=O)O)c3cc(*)ccc3/c(=C(\C#N)C(=O)OCC)c2c1]

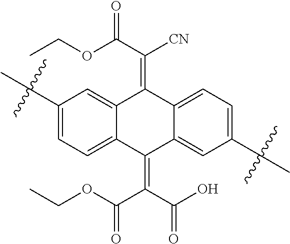 [SMILES: *c1ccc2/c(=C(/C(=O)O)C(=O)OCC)c3cc(*)ccc3/c(=C(\C#N)C(=O)OCC)c2c1]

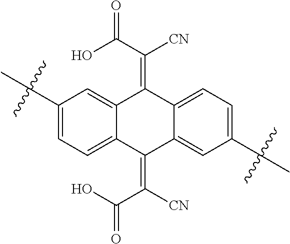 [SMILES: *c1ccc2/c(=C(/C#N)C(=O)O)c3cc(*)ccc3/c(=C(\C#N)C(=O)O)c2c1]

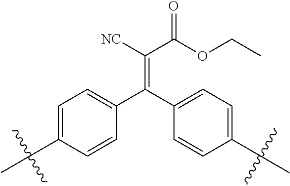 [SMILES: *c1ccc(C(=C(C#N)C(=O)OCC)c2ccc(*)cc2)cc1]

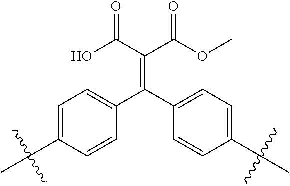 [SMILES: *c1ccc(C(=C(C(=O)O)C(=O)OC)c2ccc(*)cc2)cc1]

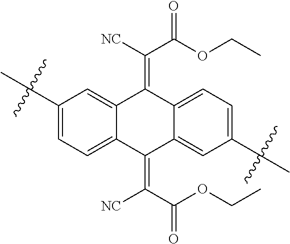 [SMILES: *c1ccc2/c(=C(/C#N)C(=O)OCC)c3cc(*)ccc3/c(=C(\C#N)C(=O)OCC)c2c1]

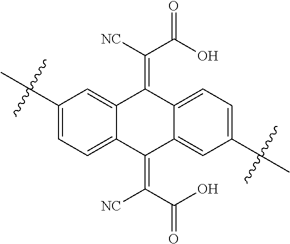 [SMILES: *c1ccc2/c(=C(/C#N)C(=O)O)c3cc(*)ccc3/c(=C(\C#N)C(=O)O)c2c1]

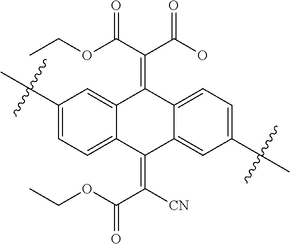 [SMILES: *c1ccc2/c(=C(/C#N)C(=O)OCC)c3cc(*)ccc3/c(=C(\C(=O)O)C(=O)OCC)c2c1]

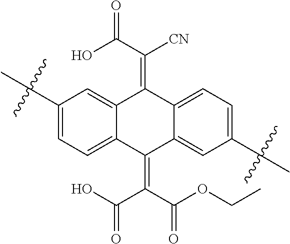 [SMILES: *c1ccc2/c(=C(\C(=O)O)C(=O)OCC)c3cc(*)ccc3/c(=C(\C#N)C(=O)O)c2c1]

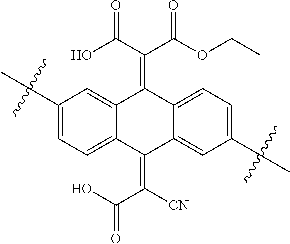 [SMILES: *c1ccc2/c(=C(/C(=O)O)C(=O)OCC)c3cc(*)ccc3/c(=C(/C#N)C(=O)O)c2c1]

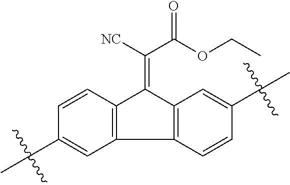 [SMILES: *c1ccc2c(c1)/C(=C(/C#N)C(=O)OCC)c1ccc(*)cc1-2]

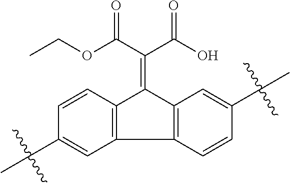 [SMILES: *c1ccc2c(c1)/C(=C(\C(=O)O)C(=O)OCC)c1ccc(*)cc1-2]

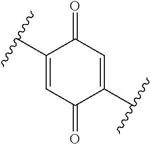 [SMILES: *C1=CC(=O)C(*)=CC1=O]

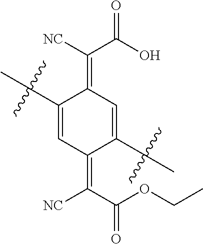 [SMILES: *c1c/c(=C(/C#N)C(=O)O)c(*)c/c1=C(\C#N)C(=O)OCC]

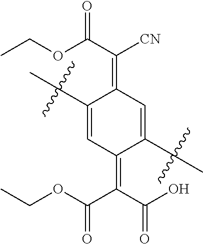 [SMILES: *c1c/c(=C(/C(=O)O)C(=O)OCC)c(*)c/c1=C(\C#N)C(=O)OCC]

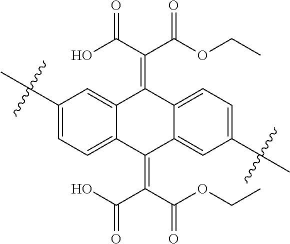 [SMILES: *c1ccc2/c(=C(/C(=O)O)C(=O)OCC)c3cc(*)ccc3/c(=C(\C(=O)O)C(=O)OCC)c2c1]

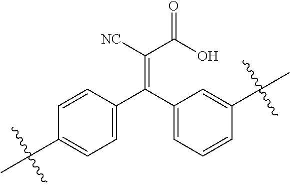 [SMILES: *c1ccc(/C(=C(\C#N)C(=O)O)c2cccc(*)c2)cc1]

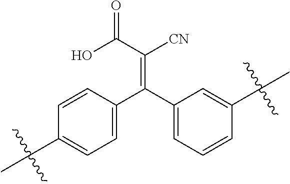 [SMILES: *c1ccc(/C(=C(/C#N)C(=O)O)c2cccc(*)c2)cc1]

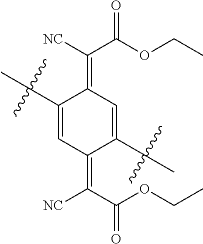 [SMILES: *c1c/c(=C(/C#N)C(=O)OCC)c(*)c/c1=C(\C#N)C(=O)OCC]

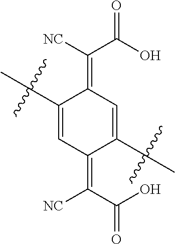 [SMILES: *c1c/c(=C(/C#N)C(=O)O)c(*)c/c1=C(\C#N)C(=O)O]

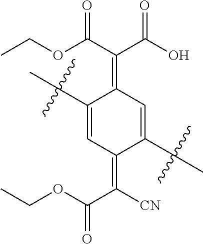 [SMILES: *c1c/c(=C(/C#N)C(=O)OCC)c(*)c/c1=C(\C(=O)O)C(=O)OCC]

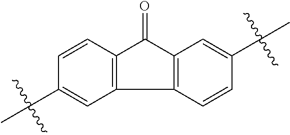 [SMILES: *c1ccc2c(c1)C(=O)c1ccc(*)cc1-2]

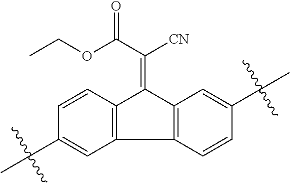 [SMILES: *c1ccc2c(c1)/C(=C(\C#N)C(=O)OCC)c1ccc(*)cc1-2]

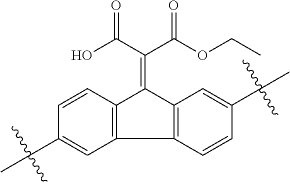 [SMILES: *c1ccc2c(c1)/C(=C(/C(=O)O)C(=O)OCC)c1ccc(*)cc1-2]

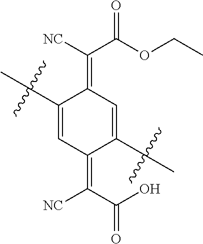 [SMILES: *c1c/c(=C(/C#N)C(=O)OCC)c(*)c/c1=C(\C#N)C(=O)O]

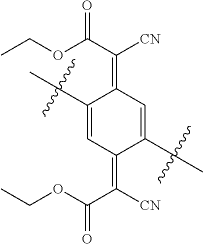 [SMILES: *c1c/c(=C(/C#N)C(=O)OCC)c(*)c/c1=C(\C#N)C(=O)OCC]

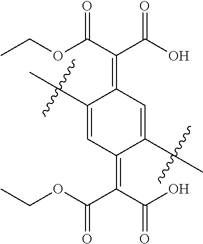 [SMILES: *c1c/c(=C(/C(=O)O)C(=O)OCC)c(*)c/c1=C(\C(=O)O)C(=O)OCC]

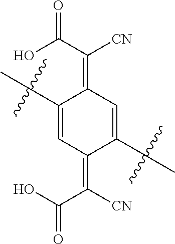 [SMILES: *c1c/c(=C(/C#N)C(=O)O)c(*)c/c1=C(\C#N)C(=O)O]

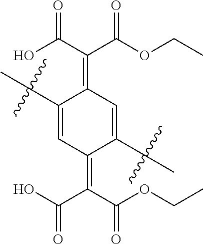 [SMILES: *c1c/c(=C(/C(=O)O)C(=O)OCC)c(*)c/c1=C(\C(=O)O)C(=O)OCC]

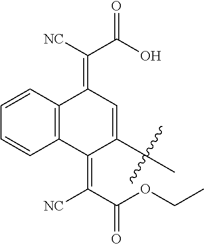 [SMILES: *c1c/c(=C(/C#N)C(=O)O)c2ccccc2/c1=C(\C#N)C(=O)OCC]

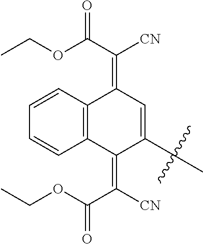 [SMILES: *c1c/c(=C(\C#N)C(=O)OCC)c2ccccc2/c1=C(/C#N)C(=O)OCC]

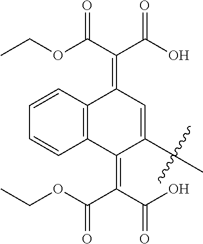 [SMILES: *c1c/c(=C(\C(=O)O)C(=O)OCC)c2ccccc2/c1=C(/C(=O)O)C(=O)OCC]

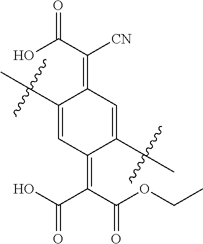 [SMILES: *c1c/c(=C(\C(=O)O)C(=O)OCC)c(*)c/c1=C(\C#N)C(=O)O]

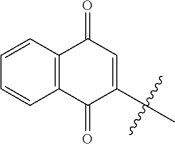 [SMILES: *C1=CC(=O)c2ccccc2C1=O]

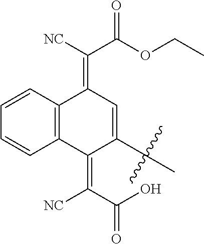 [SMILES: *c1c/c(=C(/C#N)C(=O)OCC)c2ccccc2/c1=C(\C#N)C(=O)O]

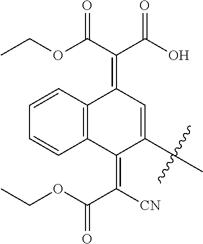 [SMILES: *c1c/c(=C(\C(=O)O)C(=O)OCC)c2ccccc2/c1=C(/C#N)C(=O)OCC]

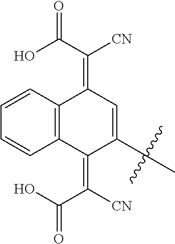 [SMILES: *c1c/c(=C(\C#N)C(=O)O)c2ccccc2/c1=C(/C#N)C(=O)O]

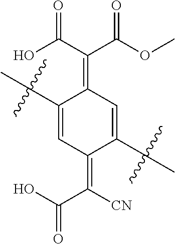 [SMILES: *c1c/c(=C(/C(=O)O)C(=O)OC)c(*)c/c1=C(/C#N)C(=O)O]

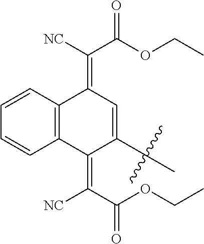 [SMILES: *c1c/c(=C(/C#N)C(=O)OCC)c2ccccc2/c1=C(\C#N)C(=O)OCC]

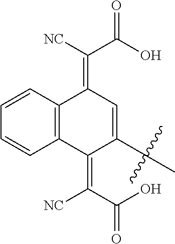 [SMILES: *c1c/c(=C(/C#N)C(=O)O)c2ccccc2/c1=C(\C#N)C(=O)O]

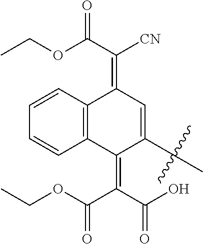 [SMILES: *c1c/c(=C(\C#N)C(=O)OCC)c2ccccc2/c1=C(/C(=O)O)C(=O)OCC]

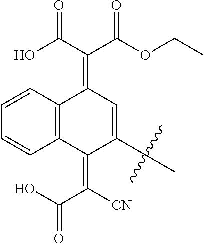 [SMILES: *c1c/c(=C(/C(=O)O)C(=O)OCC)c2ccccc2/c1=C(/C#N)C(=O)O]

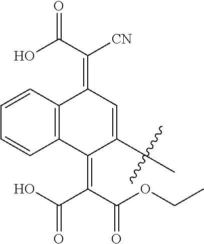 [SMILES: *c1c/c(=C(\C#N)C(=O)O)c2ccccc2/c1=C(\C(=O)O)C(=O)OCC]

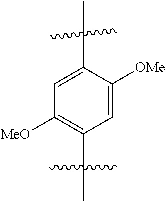 [SMILES: *c1cc(OC)c(*)cc1OC]

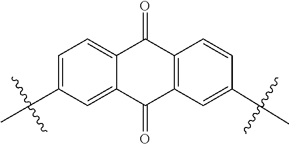 [SMILES: *c1ccc2c(c1)C(=O)c1cc(*)ccc1C2=O]

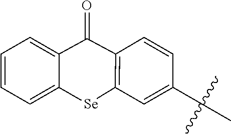 [SMILES: *c1ccc2c(=O)c3ccccc3[se]c2c1]

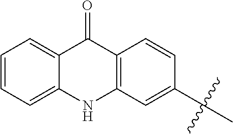 [SMILES: *c1ccc2c(=O)c3ccccc3[nH]c2c1]

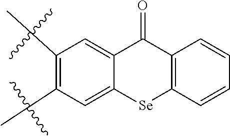 [SMILES: *c1cc2[se]c3ccccc3c(=O)c2cc1*]

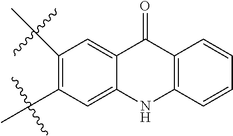 [SMILES: *c1cc2[nH]c3ccccc3c(=O)c2cc1*]

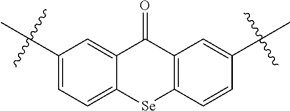 [SMILES: *c1ccc2[se]c3ccc(*)cc3c(=O)c2c1]

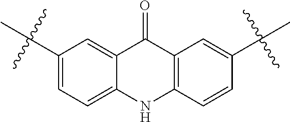 [SMILES: *c1ccc2[nH]c3ccc(*)cc3c(=O)c2c1]

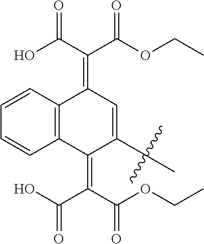 [SMILES: *c1c/c(=C(/C(=O)O)C(=O)OCC)c2ccccc2/c1=C(\C(=O)O)C(=O)OCC]

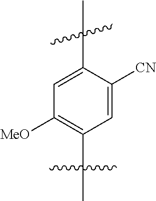 [SMILES: *c1cc(OC)c(*)cc1C#N]

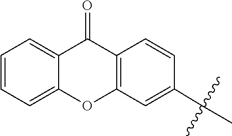 [SMILES: *c1ccc2c(=O)c3ccccc3oc2c1]

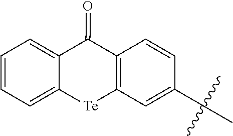

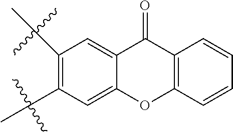 [SMILES: *c1cc2oc3ccccc3c(=O)c2cc1*]

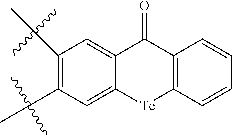

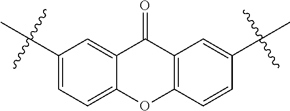 [SMILES: *c1ccc2oc3ccc(*)cc3c(=O)c2c1]

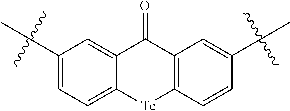

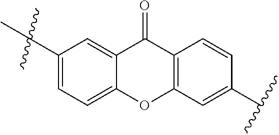 [SMILES: *c1ccc2c(=O)c3cc(*)ccc3oc2c1]

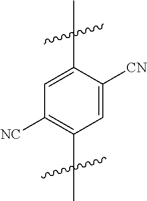 [SMILES: *c1cc(C#N)c(*)cc1C#N]

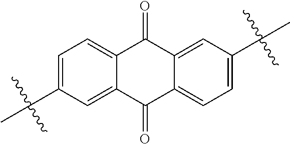 [SMILES: *c1ccc2c(c1)C(=O)c1ccc(*)cc1C2=O]

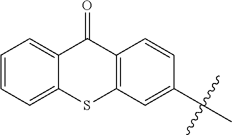 [SMILES: *c1ccc2c(=O)c3ccccc3sc2c1]

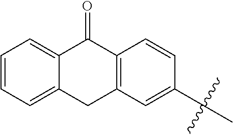 [SMILES: *c1ccc2c(c1)Cc1ccccc1C2=O]

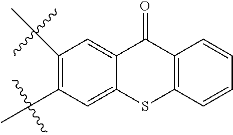 [SMILES: *c1cc2sc3ccccc3c(=O)c2cc1*]

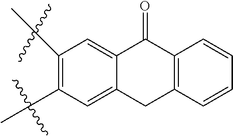 [SMILES: *c1cc2c(cc1*)C(=O)c1ccccc1C2]

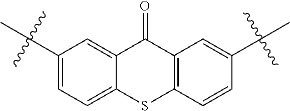 [SMILES: *c1ccc2sc3ccc(*)cc3c(=O)c2c1]

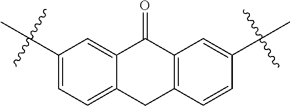 [SMILES: *c1ccc2c(c1)C(=O)c1cc(*)ccc1C2]

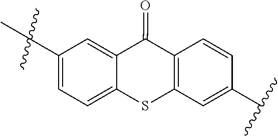 [SMILES: *c1ccc2c(=O)c3cc(*)ccc3sc2c1]

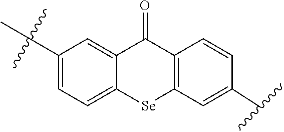 [SMILES: *c1ccc2c(=O)c3cc(*)ccc3[se]c2c1]

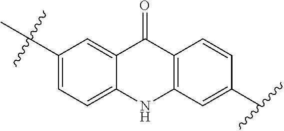 [SMILES: *c1ccc2c(=O)c3cc(*)ccc3[nH]c2c1]

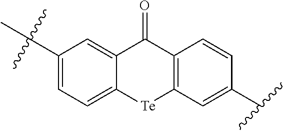

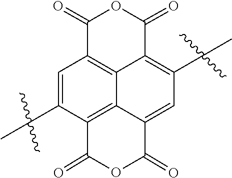 [SMILES: *c1cc2c3c(c(*)cc4c3c1C(=O)OC4=O)C(=O)OC2=O]

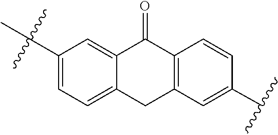 [SMILES: *c1ccc2c(c1)Cc1ccc(*)cc1C2=O]

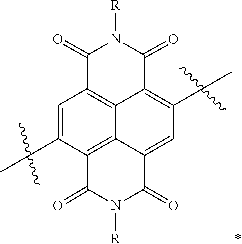

*R can be any compound with an amino group, including alkyl (saturated and unsaturated) and aromatic/non aromatic rings, including those with open-shell character.

In some embodiments, each D (e.g., D¹ and/or D²) is independently an electron donator. In some embodiments, each D is a π-conjugated system comprising an electron-donating group, such as —OH, an amine (primary (—NH₂), secondary (—NH—), or tertiary), an ether (—O—), an amide (—NHC(O)alkyl), or an alkyl.

In some embodiments, each D (e.g., D¹ and/or D²) is independently of formula (III-A):

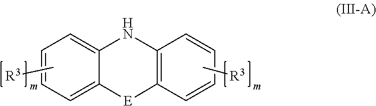

or a salt thereof, wherein
   E is absent, a bond, O, S, Se, or Te;
   each R³ is independently C₁-C₁₂ alkyl, C₁-C₆ alkoxy, 5-6 membered heteroaryl, or aryl,
   wherein aryl is optionally substituted with C₁-C₁₂ alkyl or C₁-C₆ alkoxy; and
   each m is independently 1 or more, for example 1, 2, 3, or 4. In some embodiments, alkyl groups can be halogenated.

In some embodiments, D (e.g., D¹ and/or D²) is of formula (III-A), or a salt thereof, wherein
E is absent, a bond, O, S, Se, or Te;
each R³ is independently C₁-C₁₂ alkyl, C₁-C₆ alkoxy, 5-6 membered heteroaryl, or aryl, wherein aryl is optionally substituted with C₁-C₁₂ alkyl or C₁-C₆ alkoxy; and
each m is independently 0, 1, 2, 3, or 4.

In some embodiments, E is absent, a bond, O, S, or Se. In some embodiments, E is absent. For example, when E is absent, D is of the formula

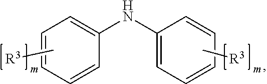

or a salt thereof, wherein R³ and m are as defined herein. In some embodiments, E is a bond. In some embodiments, E is O. In some embodiments, E is S. In some embodiments, E is Se. In some embodiments, E is Te.

When an A is attached to a D, a hydrogen on A is replaced with the bond to D. For example, in the formula III-A, a bond to D can be formed at a location on the phenyl ring by replacing a phenyl hydrogen or on the amine by replacing the amino hydrogen. When an A is attached to an L a hydrogen on A is replaced with the bond to L.

In some embodiments, each R³ is independently C₁-C₁₂ alkyl, C₁-C₆ alkoxy, 5-6 membered heteroaryl, or aryl wherein the aryl is optionally substituted with C₁-C₆ alkyl or C₁-C₆ alkoxy. In some embodiments, one R³ is C₁-C₁₂ alkyl. In some embodiments, two R³ are C₁-C₁₂ alkyl. In some embodiments, one R³ is CH₃. In some embodiments, two R³ are CH₃. In some embodiments, one R³ is C₁-C₆ alkoxy. In some embodiments, one R³ is aryl wherein the aryl is optionally substituted with C₁-C₆ alkyl. In some embodiments, two R³ are aryl wherein the aryl is optionally substituted with C₁-C₆ alkyl. In some embodiments, one R³ is phenyl substituted with one CH₃. In some embodiments, two R³ are phenyl substituted with one CH₃. In some embodiments, two R³ are C₁-C₁₂ alkyl and two R³ are aryl wherein the aryl is optionally substituted with C₁-C₆ alkyl. In some embodiments, two R³ are CH₃ and two R³ are phenyl substituted with one CH₃.

In some embodiments, each m is independently 0, 1, 2, 3, or 4. In some embodiments, each m is independently 0 or 1. In some embodiments, each m is independently 1 or 2.

In some embodiments, each D (e.g., D¹ and/or D²) is independently of formula (III-B):

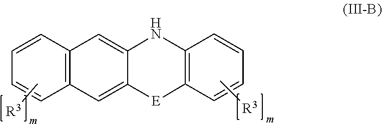

or a salt thereof, wherein
   E is absent, a bond, O, S, or Se;
   each R³ is independently C₁-C₆ alkyl, C₁-C₆ alkoxy, 5-6 membered heteroaryl, or aryl, wherein aryl is optionally substituted with C₁-C₆ alkyl or C₁-C₆ alkoxy; and
   each m is independently 1, 2, or 3.

In some embodiments, D (e.g., D¹ and/or D²) is of formula (III-B), or a salt thereof, wherein
E is absent, a bond, O, S, or Se;
each R³ is independently C₁-C₁₂ alkyl, C₁-C₆ alkoxy, 5-6 membered heteroaryl, or aryl, wherein aryl is optionally substituted with C₁-C₁₂ alkyl or C₁-C₆ alkoxy; and
each m is independently 0, 1, 2, or 3.

In some embodiments, each D (e.g., D¹ and/or D²) is independently of formula (III-C):

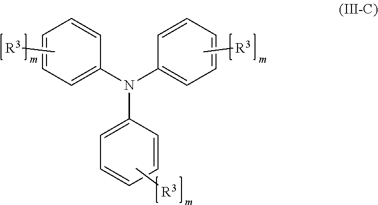

or a salt thereof, wherein
   each R³ is independently C₁-C₆ alkyl, C₁-C₆ alkoxy, 5-6 membered heteroaryl, or aryl, wherein aryl is optionally substituted with C₁-C₆ alkyl or C₁-C₆ alkoxy; and
   each m is independently 1, 2, or 3.

In some embodiments, D (e.g., D¹ and/or D²) is of formula (III-C), or a salt thereof, wherein
each R³ is independently C₁-C₁₂ alkyl, C₁-C₆ alkoxy, 5-6 membered heteroaryl, or aryl, wherein aryl is optionally substituted with C₁-C₁₂ alkyl or C₁-C₆ alkoxy; and
each m is independently 0, 1, 2, or 3.

In some embodiments, E is absent, a bond, O, S, or Se. In some embodiments, E is absent. In some embodiments, E is a bond. In some embodiments, E is O. In some embodiments, E is S. In some embodiments, E is Se. In some embodiments, E is Te.

In some embodiments, each R³ is independently C₁-C₆ alkyl. In some embodiments, one R³ is C₁-C₆ alkyl. In some embodiments, two R³ are C₁-C₆ alkyl. In some embodiments, three R³ are C₁-C₆ alkyl. In some embodiments, one R³ is CH₃. In some embodiments, two R³ are CH₃. In some embodiments, three R³ are CH₃.

In some embodiments, each m is independently 0, 1, 2, 3, or 4. In some embodiments, each m is independently 0 or 1. In some embodiments, each m is independently 1 or 2.

In some embodiments, each D (e.g., D¹ and/or D²) is independently a C₆-C₁₄ aryl ring system substituted with or comprising one or more electron-donating group, such as —OH, an amine (primary (—NH₂), secondary (—NH—), or tertiary), an ether (—O—), an amide (—NHC(O)alkyl), or an alkyl.

In some embodiments, each D is the same. In some embodiments, each D is different. In some embodiments, each D is a carbazole optionally substituted with 1 to 3 alkyl groups.

In some embodiments, each D (e.g., D¹ and/or D²) is independently a carbazole. In some embodiments, each D is of the formula:

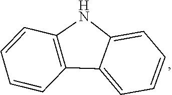 [SMILES: c1ccc2c(c1)[nH]c1ccccc12]

or a salt thereof.

In some embodiments, each D is of the formula:

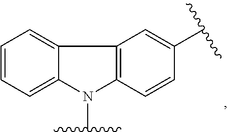 [SMILES: *c1ccc2c(c1)c1ccccc1n2*]

or a salt thereof.

In some embodiments, each D (e.g., D¹ and/or D²) is independently selected from Table B.

TABLE B
Electron Donor (D) Group Structures

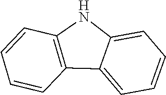 [SMILES: c1ccc2c(c1)[nH]c1ccccc12]

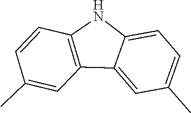

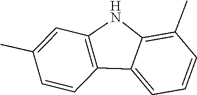

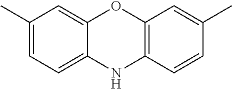

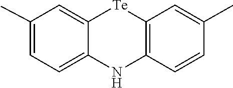

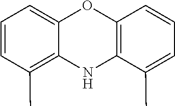 [SMILES: Cc1cccc2c1Nc1c(C)cccc1O2]

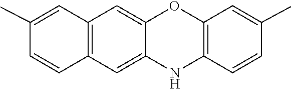

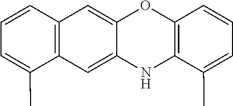 [SMILES: Cc1cccc2c1Nc1cc3c(C)cccc3cc1O2]

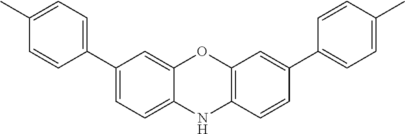

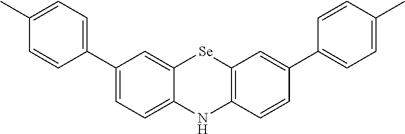

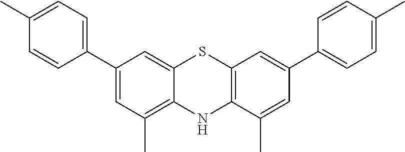

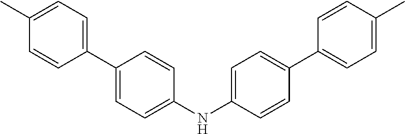

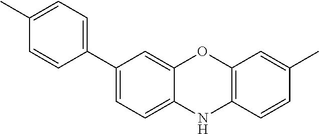

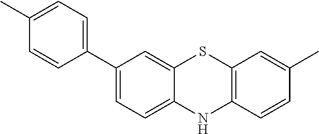

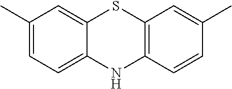

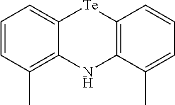

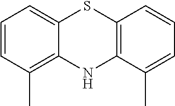 [SMILES: Cc1cccc2c1Nc1c(C)cccc1S2]

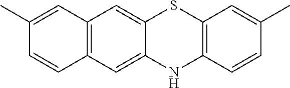

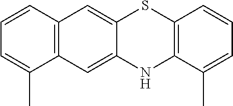 [SMILES: Cc1cccc2c1Nc1cc3c(C)cccc3cc1S2]

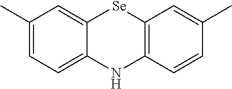

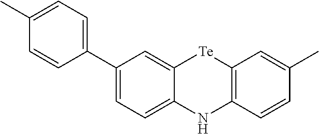

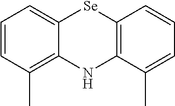 [SMILES: Cc1cccc2c1Nc1c(C)cccc1[Se]2]

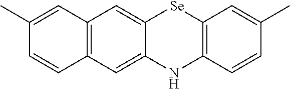

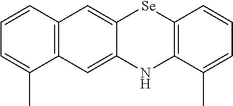 [SMILES: Cc1cccc2c1Nc1cc3c(C)cccc3cc1[Se]2]

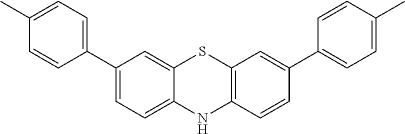

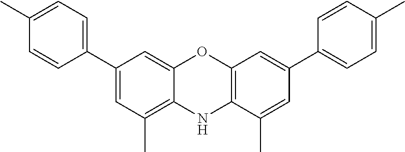

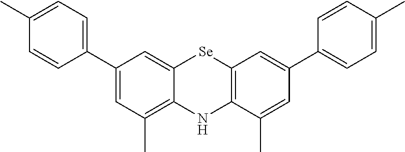

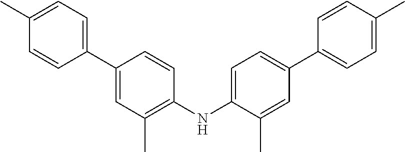

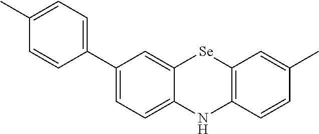

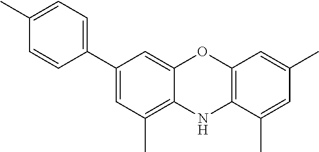

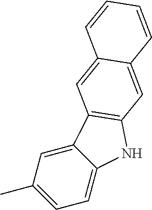 [SMILES: Cc1ccc2[nH]c3cc4ccccc4cc3c2c1]

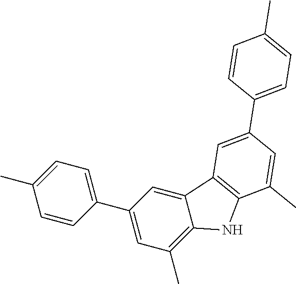

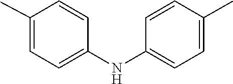

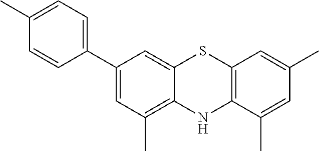

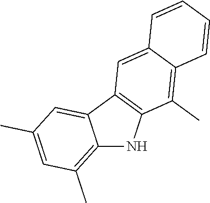 [SMILES: Cc1cc(C)c2[nH]c3c(C)c4ccccc4cc3c2c1]

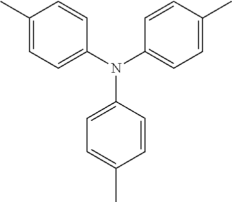

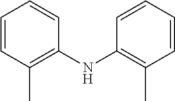

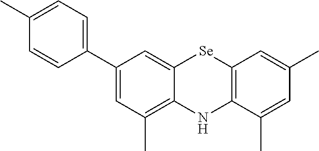

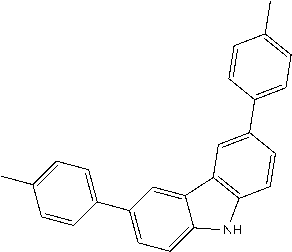 [SMILES: Cc1ccc(-c2ccc3[nH]c4ccc(-c5ccc(C)cc5)cc4c3c2)cc1]

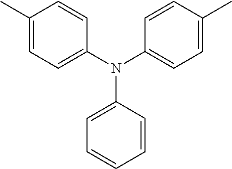

In some embodiments, each D (e.g., D¹ and/or D²) is independently selected from Table B-1.

TABLE B-1
Electron Donor (D) Group Structures

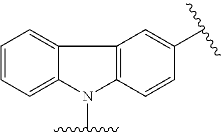 [SMILES: *c1ccc2c(c1)c1ccccc1n2*]

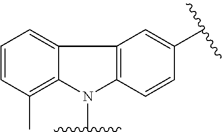 [SMILES: *c1ccc2c(c1)c1cccc(C)c1n2*]

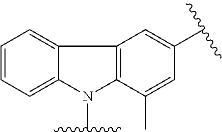 [SMILES: *c1cc(C)c2c(c1)c1ccccc1n2*]

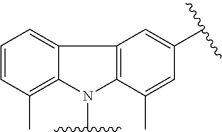 [SMILES: *c1cc(C)c2c(c1)c1cccc(C)c1n2*]

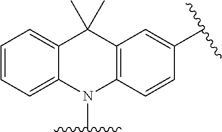 [SMILES: *c1ccc2c(c1)C(C)(C)c1ccccc1N2*]

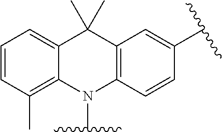 [SMILES: *c1ccc2c(c1)C(C)(C)c1cccc(C)c1N2*]

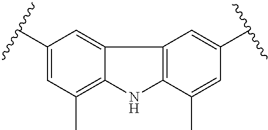 [SMILES: *c1cc(C)c2[nH]c3c(C)cc(*)cc3c2c1]

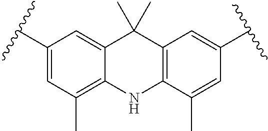 [SMILES: *c1cc(C)c2c(c1)C(C)(C)c1cc(*)cc(C)c1N2]

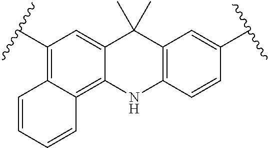 [SMILES: *c1ccc2c(c1)C(C)(C)c1cc(*)c3ccccc3c1N2]

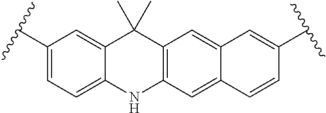 [SMILES: *c1ccc2c(c1)C(C)(C)c1cc3cc(*)ccc3cc1N2]

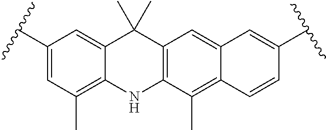 [SMILES: *c1cc(C)c2c(c1)C(C)(C)c1cc3cc(*)ccc3c(C)c1N2]

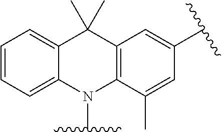 [SMILES: *c1cc(C)c2c(c1)C(C)(C)c1ccccc1N2*]

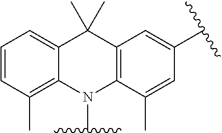 [SMILES: *c1cc(C)c2c(c1)C(C)(C)c1cccc(C)c1N2*]

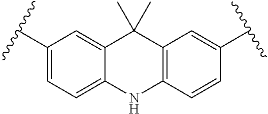 [SMILES: *c1ccc2c(c1)C(C)(C)c1cc(*)ccc1N2]

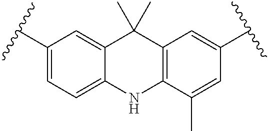 [SMILES: *c1ccc2c(c1)C(C)(C)c1cc(*)cc(C)c1N2]

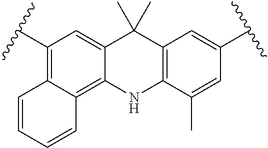 [SMILES: *c1cc(C)c2c(c1)C(C)(C)c1cc(*)c3ccccc3c1N2]

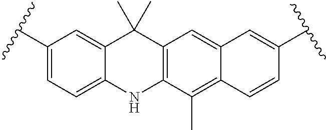 [SMILES: *c1ccc2c(c1)C(C)(C)c1cc3cc(*)ccc3c(C)c1N2]

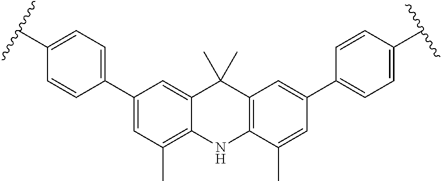 [SMILES: *c1ccc(-c2cc(C)c3c(c2)C(C)(C)c2cc(-c4ccc(*)cc4)cc(C)c2N3)cc1]

In some embodiments, the compound comprises a portion (e.g., an A-D, such as A¹-D¹, D¹-A², or A²-D²) having the formula IV-A

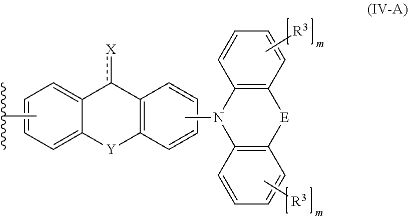

or a salt thereof, wherein
   X is O or CR¹R²;
   Y is absent, a bond, C(O), or CR¹R².
   E is absent, a bond, O, S, Se, or Te;
   each R¹ is independently CN, C(O)OH, or C(O)O—C₁-C₆ alkyl;
   each R² is independently CN, C(O)OH, or C(O)O—C₁-C₆ alkyl;
   each R³ is independently C₁-C₁₂ alkyl, C₁-C₆ alkoxy, 5-6 membered heteroaryl, or aryl wherein the aryl is optionally substituted with C₁-C₁₂ alkyl or C₁-C₆ alkoxy; and
   each m is independently 0, 1, 2, 3, or 4.

In some embodiments, each L (e.g., L¹ and/or L²) is independently a R-conjugated linker. In some embodiments, each L is an arylene-alkenylene (e.g., C₆-C₁₀ arylene-alkenylene, such as phenylene-C₂-C₆ alkenylene). In some embodiments, each L is an aryl-alkene (e.g., C₆-C₁₀ aryl-alkene such as phenyl-C₂-C₆ alkene). In some embodiments, each L is vinyl benzene.

In some embodiments, each L (e.g., L¹ and/or L²) is independently represented by the formula:

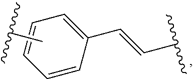

such as

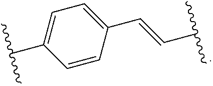 [SMILES: */C=C/c1ccc(*)cc1]

In some embodiments, each L is the same. In some embodiments, each L is different.

In some embodiments, each B (e.g., B¹ and/or B²) independently comprises an ionic group and/or a water solubilizing group. The water solubilizing group, for example, can be an ionic group. In some embodiments, each B independently comprises a quaternary amine/ammonium ion.

In some embodiments, each B (e.g., B¹ and/or B²) is independently an aryl (e.g., a C₆-C₁₀ aryl, such as phenyl) substituted with one to four alkyl (e.g., C₁-C₁₀ alkyl) or alkoxy (e.g., C₁-C₁₀ alkoxy) chains, each of which may be substituted with a water solubilizing group (e.g., a tertiary amine substituent). A group that includes a tertiary amine substituent (e.g., a tertiary amine bound to a carbon atom of an alkyl or an alkoxy group) can refer to group that comprises a quaternary amine. In some embodiments, each B is independently an aryl (e.g., a C₆-C₁₀ aryl, such as phenyl) substituted with one to four alkyl chains (e.g., C₁-C₁₀ alkyl) substituted with a water solubilizing group (e.g., a tertiary amine substituent). In some embodiments, each B is independently an aryl (e.g., a C₆-C₁₀ aryl such as phenyl) substituted with one to four alkoxy chains (e.g., C₁-C₁₀ alkoxy) substituted with a water solubilizing group (e.g., a tertiary amine substituent).

In some embodiments, each B (e.g., B¹ and/or B²) is independently phenyl substituted with three alkoxy chains (e.g., C₁-C₁₀ alkoxy) substituted with a water solubilizing group (e.g., a tertiary amine substituent). In some embodiments, the alkoxy-water solubilizing groups include a tertiary amine substituent (e.g., an amine substituted with three alkyl groups (e.g., C₁-C₆ alkyl, such as methyl)). A group that includes a tertiary amine substituent (e.g., a tertiary amine bound to a carbon atom of an alkoxy group) can refer to group that comprises a quaternary amine. In some embodiments, the alkoxy-water solubilizing groups include a quaternary amine. In some embodiments, each B is phenyl substituted with three alkoxy chains (e.g., C₁-C₁₀ alkoxy), each substituted with an ammonium ion (e.g., a quaternary amine/ammonium ion). In some embodiments, each B is phenyl substituted with three alkoxy chains (e.g., C₁-C₁₀ alkoxy), each substituted with a charged amine (e.g., a quaternary amine/ammonium ion).

In some embodiments, each B (e.g., B¹ and/or B²) is independently of the formula:

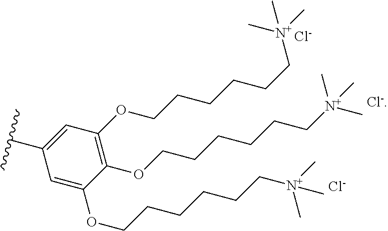

In some embodiments, each B is different. In some embodiments, each B is the same.

In some embodiments, m is 0 or 1. In some embodiments, m is 0. In some embodiments, m is 1.

In some embodiments n is 0 or 1. In some embodiments, n is 0. In some embodiments, n is 1.

In some embodiments, o is 0 or 1. In some embodiments, o is 0. In some embodiments, o is 1.

In some embodiments, p is 0 or 1. In some embodiments, p is 0. In some embodiments, p is 1.

In some embodiments, m, n, o, and p are selected from: m is 0 and n, o, and p are each 1; n is 1 and m, o, and p are each 0; each of m, n, o, and p are 0; and each of m, n, o, and p are 1.

In some embodiments, m is 0 and n, o, p are each 1. In some embodiments, when m is 0 and n, o, p are each 1, then formula (I) is B¹-L¹-D¹-A²-D²-L²-B².

In some embodiments, n is 1 and m, o, p are each 0. In some embodiments, when n is 1 and m, o, p are each 0, then formula (I) is B¹-L¹-D¹-A²-D.

In some embodiments, each of m, n, o, and p are 0. In some embodiments, when m, n, o, p are each 0, then formula (I) is B¹-L¹-D¹-A².

In some embodiments, each of m, n, o, and p are 1. In some embodiments, when m, n, o, p are each 1, then formula (I) is B¹-L¹-A¹-D¹-A²-D²-L²-B².

In some embodiments, the compound is cationic, anionic, or zwitterionic. In some embodiments, the compound has a net cationic charge of +1 to +12. In some embodiments, the compound has a net anionic charge of −1 to −12. The net cationic/anionic charge of the compound, for example, does not include the charge of an associated counterion.

In some embodiments, the compound is:

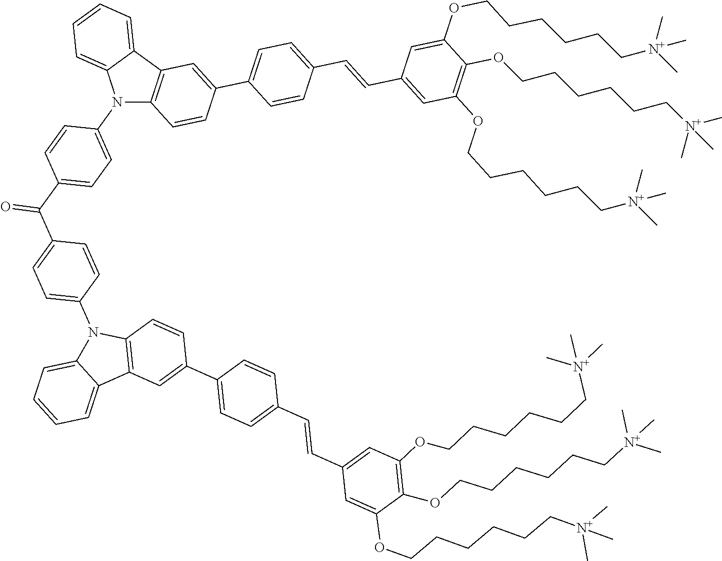 [SMILES: C[N+](C)(C)CCCCCCOc1cc(/C=C/c2ccc(-c3ccc4c(c3)c3ccccc3n4-c3ccc(C(=O)c4ccc(-n5c6ccccc6c6cc(-c7ccc(/C=C/c8cc(OCCCCCC[N+](C)(C)C)c(OCCCCCC[N+](C)(C)C)c(OCCCCCC[N+](C)(C)C)c8)cc7)ccc65)cc4)cc3)cc2)cc(OCCCCCC[N+](C)(C)C)c1OCCCCCC[N+](C)(C)C]

or a salt thereof.

In some embodiments, the compound is:

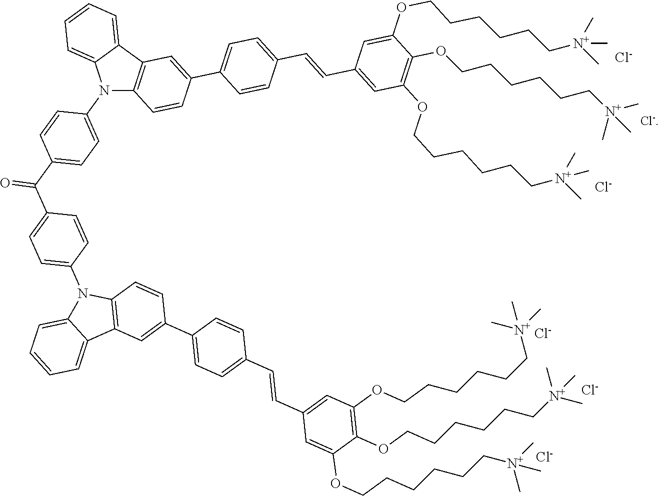 [SMILES: C[N+](C)(C)CCCCCCOc1cc(/C=C/c2ccc(-c3ccc4c(c3)c3ccccc3n4-c3ccc(C(=O)c4ccc(-n5c6ccccc6c6cc(-c7ccc(/C=C/c8cc(OCCCCCC[N+](C)(C)C)c(OCCCCCC[N+](C)(C)C)c(OCCCCCC[N+](C)(C)C)c8)cc7)ccc65)cc4)cc3)cc2)cc(OCCCCCC[N+](C)(C)C)c1OCCCCCC[N+](C)(C)C ; [Cl-] ; [Cl-] ; [Cl-] ; [Cl-] ; [Cl-]]

Those skilled in the art will recognize that the species listed or illustrated herein are not exhaustive, and that additional species within the scope of these defined terms may also be selected.

### Uses of Compounds and Compositions

In some aspects, the disclosure relates to a composition comprising a compound of the disclosure and a liposome. In some embodiments, the disclosure relates to a composition comprising a compound of the disclosure and a lipid-based carrier or lipid nanoformulation (e.g., liposome or LNP).

A variety of methods can be used for preparing the lipid carrier or lipid nanoformulation (e.g., liposomes or LNPs) described herein. Such methods are known in the art or disclosed herein, for example, the methods described in Lichtenberg and Barenholz in Methods of Biochemical Analysis, 33:337-462 (1988), which is incorporated herein by reference in its entirety. Small unilamellar vesicles (SUV, size <100 nm) can be prepared by a combination of standard methods of thin-film hydration and repeated extrusion.

Techniques for sizing the lipid carrier or lipid nanoformulations (e.g., liposomes or LNPs) to a desired size are well-known to one skilled in the art. See, e.g., U.S. Pat. No. 4,737,323, and Hope et al., Biochim. Biophys. Acta, 812: 55-65, which are incorporated by reference in their entirety.

Extrusion of lipid nanoformulations (e.g., liposomes or LNPs) through a small-pore polycarbonate membrane or an asymmetric ceramic membrane is a very effective method for reducing liposome or LNP sizes to a relatively well-defined size distribution. Typically, the suspension is cycled through the membrane one or more times until the desired liposome or LNP size distribution is achieved. The lipid-based carrier or lipid nanoformulations may be extruded through successively smaller-pore membranes, to achieve a gradual reduction in liposome or LNP size.

The lipid carrier or lipid nanoformulation may optionally include one or more coatings. In some embodiments, the lipid carrier or lipid nanoformulation (e.g., liposome or LNP) may be formulated in a capsule, film, or tablet having a coating. A capsule, film, or tablet including a composition described herein may have any useful size, tensile strength, hardness, or density.

In some aspects, the disclosure relates to a composition comprising a compound of the disclosure and a nucleic acid (e.g., DNA or RNA).

In some aspects, the disclosure features a method of delivering a compound to a cell comprising contacting a cell with a composition (e.g., a liposome) of the disclosure or a compound of the disclosure or a pharmaceutical composition thereof.

In some aspects, the disclosure features a method of quantifying an amount (e.g., a concentration) of a nucleic acid (e.g., DNA or RNA) in a biological sample, comprising:
(i) contacting a biological sample with a compound of the disclosure;
(ii) exposing the biological sample and the compound to light of a first wavelength; and
(iii) detecting an emission of a second wavelength from the compound in the biological sample.

In yet another aspect, the disclosure features a method of imaging a biological sample comprising
(i) contacting a biological sample (e.g., a cell) with a composition of the disclosure or a compound of the disclosure;
(ii) exposing the biological sample to light having a first wavelength; and
(iii) detecting an emission of a second wavelength from the biological sample.

In yet another aspect, the disclosure features a method of imaging a biological sample comprising
(i) contacting a biological sample with a compound of the disclosure;
(ii) exposing the biological sample and the compound to light having a first wavelength; and
(iii) detecting an emission of a second wavelength from the compound in the biological sample.

In some embodiments, the method is performed at about room temperature (e.g., about 20° C. to about 25° C.).

In some embodiments, the method is performed under aqueous conditions.

In some embodiments, the method is performed in the presence of a liposome.

In some embodiments, the biological sample comprises a nucleic acid, such as DNA or RNA. In some embodiments, the biological sample comprises DNA or RNA. In some embodiments, the biological sample comprises a lipid bilayer.

In some embodiments, the method further comprises quantifying an amount of a nucleic acid. In some embodiments, the method further comprises quantifying an amount of DNA or RNA.

In some embodiments, the method further comprises generating a spin-correlated radical pair (SCRP). For example, the SCRP may be generated by introducing a partial break in π-conjugation between the electron donor (e.g., carbazole) and the acceptor (e.g., benzophenone) in the compounds of the disclosure (e.g., via a nitrogen-carbon junction). In some embodiments, the radical pair comprises an excited singlet state and an excited triplet state, and a minimized singlet-triplet energy gap (ΔE_{ST}). In some embodiments, the ΔE_{ST} is about 0.3 eV (~2400 cm⁻¹) or lower, such as about 0.01 eV to about 0.30 eV. For example, an ΔE_{ST}<0.3 eV (~2400 cm⁻¹) is consistent with the Boltzmann energetic criteria for thermally activated spin mixing in chromophores exhibiting TADF characteristics.

In some embodiments, the SCRP is generated when the compound is in a diffuse environment, in the presence of freely diffuse biomolecules, or in contact with a nucleic acid. In some embodiments, the SCRP is generated in the presence of a nucleic acid and/or freely diffuse biomolecules.

In some embodiments, the SCRP is not generated when the compound is in a restricted environment or intercalated in a lipid bilayer.

In some embodiments, the method comprises contacting the compounds of the disclosure with a nucleic acid (e.g., DNA or RNA) and/or a lipid bilayer. In some embodiments, compounds of the disclosure interact with the nucleic acid to form a twisted intramolecular charge-transfer (TICT) geometry. In some embodiments, compounds of the disclosure intercalate into lipid bilayers to form a planar geometry.

In some embodiments, the SCRP is sensitive to the magnetic field of a nucleic acid and the electric field of freely diffuse biomolecules.

In some embodiments, the first wavelength is the absorption wavelength of a compound of the disclosure. In some embodiments, the first wavelength is about 350 nm. In some embodiments, the first wavelength is about 351 nm. In some embodiments, the first wavelength is about 315 nm. In some embodiments, the first wavelength may be from about 315 nm to about 460 nm. In some embodiments, the first wavelength may be about 315 nm, about 325 nm, about 340 nm, about 350 nm, about 365 nm, about 375 nm, about 380 nm, about 390 nm, about 400 nm, about 415 nm, about 420 nm, about 430 nm, about 440 nm, about 450 nm, or about 460 nm.

In some embodiments, the second wavelength is the emission wavelength of a compound of the disclosure. In some embodiments, the second wavelength is about 520 nm to 530 nm. In some embodiments, the second wavelength is about 520 nm, about 521 nm, about 522 nm, about 523 nm, about 524 nm, about 525 nm, about 526 nm, about 527 nm, about 528 nm, about 529 nm, and about 530 nm.

In some embodiments, the first wavelength is about 350 nm and the second wavelength is about 520 nm. In some embodiments, the first wavelength is about 351 nm, and the second wavelength is about 522 nm. In some embodiments, the first wavelength is about 351 nm, and the second wavelength is about 528 nm.

In some embodiments, the compounds of the disclosure are fluorescent. In some embodiments, the compounds of the disclosure are phosphorescent. In some embodiments, the compounds of the disclosure exhibit a thermally activated delayed fluorescent character. In some embodiments, the compounds of the disclosure have both fluorescent and phosphorescent properties. In some embodiments, the compounds of the disclosure have a minimal singlet (S₁)-triplet (T₁) energy gap. In some embodiments, the compounds of the disclosure have a triplet excited state. In some embodiments, the compounds of the disclosure have a doublet excited state. In some embodiments, the compounds of the disclosure have a singlet excited state. In some embodiments, the compounds of the disclosure have a photogenerated excited state.

In some embodiments, the compounds of the disclosure comprise two spatially separated, spin-entangled radicals. In some embodiments, the compounds of the disclosure comprise a spin-correlated radical pair (SCRP). For example, the compounds of the disclosure may have a SCRP in a singlet state (¹SCRP) and/or a triplet state (³SCRP). In some embodiments, the SCRP is polarizable. The emergence of polarizable SCRP behavior at room temperature may be provided by introducing a partial break in π-conjugation between the electron donor (e.g., carbazole) and the acceptor (e.g., benzophenone) in the compounds of the disclosure (e.g., via a nitrogen-carbon junction).

In some embodiments, the compounds of the disclosure have different electronic rates in different environments. The different environments can include a freely diffuse system, associated with DNA, or intercalated with a liposome.

In some embodiments, the compounds of the disclosure have a charge separation rate (¹k_{CS}). For example, the compounds of the disclosure may have a ¹k_{CS} of about 1×10¹² s⁻¹ to about 9×10¹² s⁻¹, such as about 5×10¹² s⁻¹.

In some embodiments, the compounds of the disclosure have a radical pair intersystem crossing rate (k_{RP-ISC}). For example, the compounds of the disclosure may have a k_{R-ISC} of about 1×10⁸ s⁻¹ to about 9×10⁸ s⁻¹, such as about 2×10⁸ s⁻¹.

In some embodiments, the compounds of the disclosure have a charge separation rate (³k_{CRT}). For example, the compounds of the disclosure may have a ³k_{CRT} of about 1×10⁷ s⁻¹ to about 9×10⁶ s⁻¹, such as about 5×10⁶ s⁻¹ or about 1×10⁷ s⁻¹.

In some embodiments, the compounds of the disclosure are water-soluble.

In some embodiments, compounds of the disclosure have an emission lifetime of about 0.5 ns to about 1 ns. In some embodiments, the emission lifetime is about 0.5 ns, about 0.6 ns, about 0.7 ns, about 0.8 ns, about 0.9 ns, or about 1 ns. In some embodiments, the emission lifetime is about 0.81 ns, about 0.82 ns, about 0.83 ns, about 0.84 ns, about 0.85 ns, about 0.86 ns, about 0.87 ns, about 0.88 ns, or about 0.89 ns.

In some embodiments, the compounds of the disclosure exhibit multi-component long-lived transient states, associated with quantum decoherence. In some embodiments, the compounds of the disclosure exhibit long-lived Tₙ states. In some embodiments, the compounds of the disclosure exhibit long-lived Dₙ states, associated with quantum decoherence.

In some embodiments, compounds of the disclosure have an excited singlet state and an excited triplet state. For example, compounds of the disclosure may have a radical pair (e.g., SCRP) having a singlet state and a triplet state. In some embodiments, compounds of the disclosure have a singlet-triplet energy gap (ΔE_{ST}) of about 0.3 eV (~2400 cm⁻¹) or lower, such as about 0.01 eV to about 0.30 eV (e.g., about 0.12 eV). For example, an ΔE_{ST}<0.3 eV (~2400 cm⁻¹) is consistent with the Boltzmann energetic criteria for thermally activated spin mixing in chromophores exhibiting TADF characteristics.

In some embodiments, the compounds of the disclosure have different optical properties and electrochemical properties in different environments. The different environments can include a freely diffuse system, associated with DNA, or intercalated with a liposome.

In some embodiments, compounds of the disclosure have a fluorescence maxima (S₁). For example, compounds of the disclosure may have a S₁ of about 2.0 eV to about 3.0 eV.

In some embodiments, compounds of the disclosure have a phosphorescence maxima (T₁). For example, compounds of the disclosure may have a T₁ of about 2.0 eV to about 3.0 eV.

In some embodiments, compounds of the disclosure have a negative Rehm-Weller calculation radical pair formation value (ΔG_{CS}). For example, compounds of the disclosure may have a ΔG_{CS} of about −0.01 eV to about −0.99 eV.

In some embodiments, compounds of the disclosure can be used in biocompatible quantum sensing and spin-selective photophysical systems, including those relevant to non-aqueous quantum information science (QIS).

In some embodiments, compounds of the disclosure can interact with a nucleic acid (e.g., DNA or RNA) and/or lipid bilayers. In some embodiments, compounds of the disclosure interact with the nucleic acid to form a twisted intramolecular charge-transfer (TICT) geometry. In some embodiments, compounds of the disclosure intercalate into lipid bilayers to form a planar geometry.

The present disclosure enables one of skill in the relevant art to make and use the inventions provided herein in accordance with multiple and varied embodiments. Various alterations, modifications, and improvements of the present disclosure that readily occur to those skilled in the art, including certain alterations, modifications, substitutions, and improvements are also part of this disclosure. Accordingly, the foregoing description are by way of example to illustrate the discoveries provided herein. Furthermore, the foregoing Description and Examples are exemplary of the present invention and not limiting thereof. The scope of the invention is therefore set out in the appended claims.

Although specific embodiments of the present disclosure are herein illustrated and described in detail, the disclosure is not limited thereto. The above detailed descriptions are provided as exemplary of the present disclosure and should not be construed as constituting any limitation of the disclosure. Modifications will be obvious to those skilled in the art, and all modifications that do not depart from the spirit of the disclosure are intended to be included with the scope of the appended claims.

Additional embodiments, features, and advantages of the disclosure will be apparent from the following detailed description and through practice of the disclosure. The compositions of the present disclosure can be described as embodiments in any of the following enumerated clauses. It will be understood that any of the embodiments or clauses described herein can be used in connection with any other embodiments or clauses described herein to the extent that the embodiments do not contradict one another.
1. A compound of formula (I):

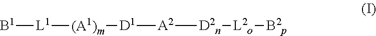

or a salt thereof, wherein
each of A¹ and A² is independently an electron acceptor;
each of D¹ and D² is independently an electron donor;
each of L¹ and L² is independently a π-conjugated linker;
each of B¹ and B² is independently an aryl substituted with one to four C₁-C₁₀ alkyl or C₁-C₁₀ alkoxy, wherein each of the alkyl or alkoxy is substituted with halogen, an ionic group, or a water-solubilizing group;
m is 0 or 1;
n is 0 or 1;
is 0 or 1; and
p is 0 or 1;
provided that:
(i) m is 0 and n, o, and p are each 1;
(ii) n is 1 and m, o, and p are each 0;
(iii) each of m, n, o, and p are 0; or
(iv) each of m, n, o, and p are 1.
2. The compound of clause 1, wherein m is 0 and n, o, and p are each 1.
3. The compound of clause 1, wherein n is 1 and m, o, and p are each 0.
4. The compound of clause 1, wherein each of m, n, o, and p are 0.
5. The compound of clause 1, wherein each of m, n, o, and p are 1.
6. The compound of any one of clauses 1 to 5, wherein each of A¹ and A² is independently of formula (II-E):

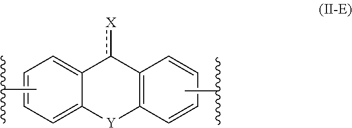

or a salt thereof, wherein
X is O or CR¹R².
Y is absent, a bond, O, S, NH, Se, Te, C(O), or CR¹R²;
each R¹ is independently H, CN, C(O)OH, or C(O)—C₁-C₆ alkyl; and
each R² is independently H, CN, C(O)OH, or C(O)—C₁-C₆ alkyl.
7. The compound of any one of clauses 1 to 6, wherein each of A¹ and A² is independently a C₆-C₁₄ aryl ring system optionally substituted with oxo, alkyene, cyano, alkoxy, acyl, halogen, or carboxyl.
8. The compound of any one of clauses 1 to 7, wherein each of A¹ and A² is independently selected from Table A.
9. The compound of any one of clauses 1 to 7, wherein each of A¹ and A² is independently a benzophenone.
10. The compound of any one of clauses 1 to 9, wherein each of D¹ and D² is independently of formula (III-A),

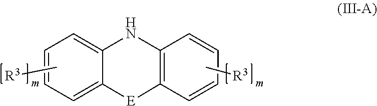

or a salt thereof, wherein
   E is absent, a bond, O, S, Se, or Te;
   each R³ is independently C₁-C₁₂ alkyl, C₁-C₆ alkoxy, 5- to 6-membered heteroaryl, or aryl, wherein aryl is optionally substituted with C₁-C₁₂ alkyl or C₁-C₆ alkoxy; and
   each m is independently 0, 1, 2, 3, or 4.
11. The compound of any one of clauses 1 to 10, wherein each of D¹ and D² is independently a carbazole, optionally substituted with 1 to 3 alkyl.
12. The compound of any one of clauses 1 to 9, wherein each of D¹ and D² is independently selected from Table B.
13. The compound of any one of clauses 1 to 11, wherein each of D¹ and D² is independently of formula:

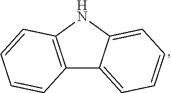 [SMILES: c1ccc2c(c1)[nH]c1ccccc12]

or a salt thereof.
14. The compound of any one of clauses 1 to 13, wherein each of L¹ and L² is independently an arylene-alkenylene.
15. The compound of any one of clauses 1 to 14, wherein each of L¹ and L² is vinyl benzene.
16. The compound of any one of clauses 1 to 15, wherein each of B¹ and B² is independently a phenyl substituted with three C₁-C₁₀ alkoxy, and wherein each alkoxy is substituted with a water solubilizing group.
17. The compound of any one of clauses 1 to 16, wherein each of B¹ and B² is independently of the formula:

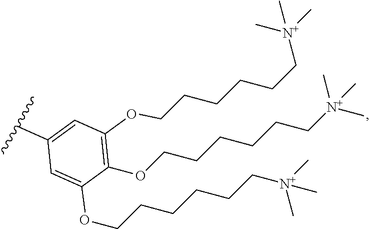 [SMILES: *c1cc(OCCCCCC[N+](C)(C)C)c(OCCCCCC[N+](C)(C)C)c(OCCCCCC[N+](C)(C)C)c1]

or a salt thereof.
18. The compound of any of one clauses 1 to 17, wherein each of B¹ and B² is independently of the formula:

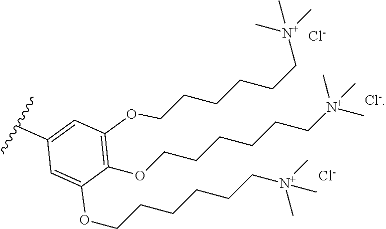 [SMILES: *c1cc(OCCCCCC[N+](C)(C)C)c(OCCCCCC[N+](C)(C)C)c(OCCCCCC[N+](C)(C)C)c1 ; [Cl-] ; [Cl-]]

19. The compound of clause 1, wherein the compound is:

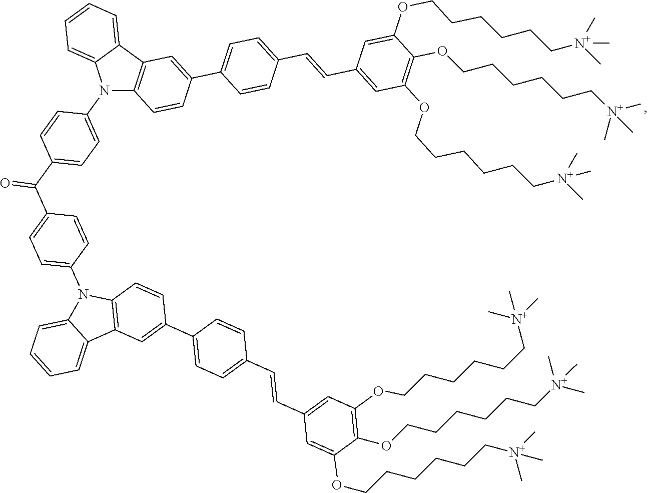 [SMILES: C[N+](C)(C)CCCCCCOc1cc(/C=C/c2ccc(-c3ccc4c(c3)c3ccccc3n4-c3ccc(C(=O)c4ccc(-n5c6ccccc6c6cc(-c7ccc(/C=C/c8cc(OCCCCCC[N+](C)(C)C)c(OCCCCCC[N+](C)(C)C)c(OCCCCCC[N+](C)(C)C)c8)cc7)ccc65)cc4)cc3)cc2)cc(OCCCCCC[N+](C)(C)C)c1OCCCCCC[N+](C)(C)C]

or a salt thereof.
20. The compound of clause 1, wherein the compound is:

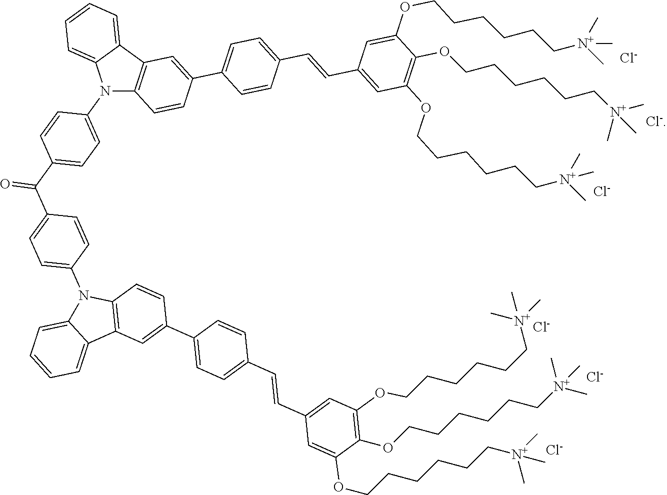 [SMILES: C[N+](C)(C)CCCCCCOc1cc(/C=C/c2ccc(-c3ccc4c(c3)c3ccccc3n4-c3ccc(C(=O)c4ccc(-n5c6ccccc6c6cc(-c7ccc(/C=C/c8cc(OCCCCCC[N+](C)(C)C)c(OCCCCCC[N+](C)(C)C)c(OCCCCCC[N+](C)(C)C)c8)cc7)ccc65)cc4)cc3)cc2)cc(OCCCCCC[N+](C)(C)C)c1OCCCCCC[N+](C)(C)C ; [Cl-] ; [Cl-] ; [Cl-] ; [Cl-] ; [Cl-]]

21. The compound of any one of clauses 1 to 20, wherein the compound has a triplet excited state.
22. The compound of any one of clauses 1 to 21, wherein the compound has spin correlated radical pair (SCRP).
23. The compound of any one of clauses 1 to 22, wherein the compound has an emission lifetime between 0.5 and 1 ns.
24. A composition comprising a compound of any one of clauses 1 to 23, and a liposome.
25. The composition of clause 24, wherein the liposome comprises phosphatidylglycerol and phosphatidylcholine.
26. A composition comprising a compound of any one of clauses 1 to 24, and a nucleic acid (e.g., DNA or RNA).
27. A method of delivering a compound to a cell comprising:
contacting a cell with the composition of clause 24 or 25 or the compound of any one of clauses 1 to 23.
28. A method of imaging a biological sample comprising:
(i) contacting a biological sample (e.g., a cell) with the composition of clause 24 or 25 or the compound of any one of clauses 1 to 23;
(ii) exposing the biological sample to light of a first wavelength; and
(iii) detecting an emission of a second wavelength in the biological sample.
29. The method of clause 28, wherein the method is performed at about room temperature.
30. The method of clause 28 or 29, wherein the method is performed under aqueous conditions.
31. The method of any one of clauses 28-30, wherein the biological sample comprises a nucleic acid (e.g., DNA or RNA).
32. The method of any one of clauses 28-31, wherein the first wavelength is about 351 nm.
33. The method of any one of clauses 28-32, wherein the second wavelength is about 490 nm to 530 nm.
34. The method of any one of clauses 28-33, wherein the first wavelength is about 351 nm and the second wavelength is about 520 nm.
35. The method of any one of clauses 28-33, wherein the method comprises contacting the nucleic acid with the compound.
36. The method of clause 35, wherein the compound interacts with the nucleic acid to form a twisted intramolecular charge-transfer (TICT) geometry.
37. The method of any one of clauses 28-36, wherein the method further comprises generating a spin-correlated radical pair (SCRP).
38. The method of clause 37, wherein the radical pair comprises an excited singlet state and an excited triplet state, and a singlet-triplet energy gap (ΔE_{ST}) of less than about 0.3 eV.

### EXAMPLES

### Materials and Methods

Nuclear Magnetic Resonance (NMR) Spectroscopy—All the NMR spectra (¹H and ¹³C-NMR) reported herein have been recorded using a Bruker 500 MHz Avance Neo NMR instrument. The NMR samples were submitted using deuterated solvents such as —CDCl₃ and DMSO-d₆ purchased from Sigma-Aldrich. The spectra were plotted in Mestre Nova software.

Quantum Chemical Simulations—The ground state optimization and the investigation of electronic structure can be performed using a Gaussian 16 software package.

UV-Vis Spectroscopy—The absorption spectra were recorded using a Cary 8454 Uv-Vis (Agilent, Santa Clara, CA) with 1×PBS (phosphate buffer saline; pH 7.4) and 1×TRIS (Tris aminomethane; pH 7.4) as solvents. The 1×PBS (—Ca and —Mg) was procured from Thermo Fischer Scientific and was used without any further purification. The 1×TRIS was prepared by dissolving the calculated amount of TRIS HCl (obtained from VWR) in Ultra Pure water.

Steady-State and Time-Resolved Emission Spectroscopy—The steady-state emission spectra reported in this study were recorded using photoluminescence spectrometer (FLS-1000) from Edinburgh Instruments upon excitation at 380 nm and with slit width 2 nm both for excitation and emission in Ultra Pure, 1×PBS and 1×TRIS as solvents. Fluorescence lifetime studies were measured using the same instrument at λₘₐₓ of the emission spectra, using an excitation source of 375 nm wavelength and within a collection window of 200 ns. Both the emission and lifetime measurements were recorded using a long-band pass filter of 395 nm. The phosphorescence measurement was performed at 77 K temperature using the same spectrometer but with a gated detector also obtained from Edinburgh Instruments, using a multipulsed lamp of excitation 405 nm at 250 μs delay time. The 77 K temperature was achieved using a finger dewar provided by Edinburgh Instruments, filled with liquid nitrogen, and contained in quartz EPR-like tubes.

Transient Absorption Spectroscopy (TAS)—Nanosecond Transient absorption measurements were conducted over a time window spanning 0 to 400 s with a temporal resolution of 800 μs, utilizing a commercially available Eos system from Ultrafast Systems. For this purpose, Hyperion, an integrated multi-kHz ultrafast Yb-based amplified laser system, was employed. The system delivers 6 W of output power with pulse energies of 500 J, which are directed to an optical parametric amplifier (OPA) module, APOLLO-Y, tunable across the ultraviolet (UV) to near-infrared (NIR) spectral regions. The pump pulses, characterized by a pulse width of approximately 250 fs, operate at a repetition rate of 1 kHz, with the pulse energy adjusted to ~4 J/cm². The supercontinuum probe is internally generated using a photonic crystal fiber (PCF), which is pumped by a low-jitter Nd:YAG laser operating at 2 kHz. The Eos system software records and processes the spectrum of each probe pulse in pairs, one with the pump excitation and one without, while electronically controlling the delay between the pump and probe pulses for precise temporal overlap and data acquisition. For the magnetic-field sensitivity studies, a permanent magnet with an approximate surface field of ~550 mT was employed. However, due to geometric decay and the sample-to-magnet separation, the magnetic field at the pump-probe interaction region was measured to be ~270 mT using a Gaussmeter.

Femtosecond Transient Absorption Spectroscopy was conducted over a time window of 8 ns with a temporal resolution of 350 fs, utilizing the commercially available Helios system from Ultrafast Systems. For these measurements, laser pulses from the Hyperion, an integrated multi-kHz ultrafast Yb-based amplified laser system, were split into two arms. One arm was directed into an optical parametric amplifier (OPA) module, APOLLO-Y, which is tunable across the ultraviolet (UV) to near-infrared (NIR) spectral regions. The pump pulses, with a pulse width of approximately 250 fs, operated at a repetition rate of 2 kHz and had their pulse energy adjusted to ~4 J/cm². The second arm was used to generate femtosecond supercontinuum probe pulses via the Helios system. The Helios system recorded and processed the spectrum of each probe pulse in pairs, one with pump excitation and one without, while a mechanical delay stage was used to introduce precise temporal offsets between the pump and probe pulses. This configuration enabled accurate temporal overlap and facilitated reliable data acquisition.

Electron Paramagnetic Resonance (EPR)—To remove atmospheric oxygen, the samples were saturated with argon and transferred into disposable BLAUBRAND® micropipettes. The tops were sealed with hot-melt glue to prevent leakage. For initial measurements, solutions were freshly prepared just before the experiments. Samples were then irradiated in situ in the cavity of the EPR spectrometer at room temperature using focused light from a 1 kW high-pressure mercury-xenon lamp (Hanovia 977 B-1) while EPR spectra were recorded. X-band (reported at 9.8 GHz) EPR spectra were recorded using a Bruker EMXplus spectrometer with ER4119HS standard resonators, employing a 1 Gauss modulation amplitude, 100 kHz field modulation, and 1 mW microwave power. For 77 K experiments, samples of COE-CbzBP in 60% glycerol were loaded into 4 mm EPR tubes, deoxygenated using the freeze-pump-thaw method, and then sealed under vacuum. For the simulations of the COE-CbzBP:4-POBN experiments, 0.5 Gauss modulation amplitude, better resolution of the spectrum, and the simulated parameters were: Radical 1 (POBN-C-centered radical): g=2.0063; line width=0.8; Line Shape=0.3; Area=0.13; aN=15; aH:=8. Radical 2 (triplet): g=2.005; line width=4.0; Line Shape=0; Area=0.45.

Regarding the COE-CbzBP:DNA experiments, a concentration of 2 mM COE-CbzBP was used in relation to a DNA concentration of 5 mg/mL, corresponding to a 1:1 COE-CbzBP:DNA ratio. The most consistent COE-CbzBP:DNA results were obtained after creating the solution, followed by overnight aging to maximize COE-CbzBP:DNA interactions. At these concentrations, the solution looks colloidal due to the formation of nanoplexes. X-band (reported at 9.8 GHz) EPR spectra were also recorded using a Bruker EMXplus spectrometer with ER4119HS standard resonators, employing a 2 Gauss modulation amplitude and 2 mW microwave power. To minimize damage, samples were irradiated in situ in the cavity of the EPR spectrometer at room temperature using an optical fiber focused light from an individual light source module at a 385 nm wavelength (15.95 mW—LED Single Channel Touchscreen Controller for LSM LED Product Line from Ocean Optics) while EPR spectra were recorded.

Liposome formation and DNA Interfacing—DNA was purchased from Thermo Fisher (Salmon Sperm DNA, sheared—10 mg/mL—Catalog number: AM9680). The concentration of DNA used in this study was 5 mg/ml for 1 equivalent of DNA relative to COE-CbzBP, 2.5 mg/ml for 0.5 equivalent of DNA relative to COE-CbzBP, 1.25 mg/ml for 0.25 equivalent of DNA relative to COE-CbzBP, and 0.625 mg/ml for 0.25 equivalent, respectively, assuming. All lipids used in this study were purchased from Avanti Polar Lipids. 1-palmitoyl-2-oleoyl-glycero-3-phosphocholine (POPC) and 1-palmitoyl-2-oleoyl-sn-glycero-3-phospho-(1′-racglycerol) sodium (POPG) were dissolved in chloroform and mixed in a glass vial at a 3:1 molar ratio. A 1% solution of COE-CbzBP was dissolved in ethanol and added to the vial. The lipid and dye mixture in chloroform was dried under a gentle stream of argon in a fume hood, followed by further drying in a vacuum oven set to 100° C. for 30 minutes to form a thin lipid film. To prepare liposomes, the dried lipid film was rehydrated with Ultra-Pure water and 1×PBS (phosphate-buffered saline, GIBCO) to a final lipid concentration of 3.125 mg/mL, and vortexed for 1 minute. Liposomes were then extruded using an Avanti Mini Extruder with a 100 nm membrane at room temperature for 21 passes. The resulting liposome preparation was stored at 4° C. until further use. All measurements were performed using freshly prepared solutions that were sonicated for at least 1 hour prior to the measurements.

Electrochemistry—Cyclic voltammetry (CV) was performed using a Biologic VSP-300 potentiostat by sweeping the working electrode potential from Eᵢₙᵢₜᵢₐₗ=0.0 V to Eᵥₒᵣₜₑₓ=2.5 V and back to E_{final}=0.0 V. All measurements were conducted in a sealable single-chamber electrochemical cell equipped with a PTFE lid. A platinum wire was used as the reference electrode, carbon paper served as the counter electrode, and ferrocene was added as an internal standard. CV measurements were carried out on both the COE-CbzBP and its non-quaternized molecular congener. Due to their poor solubility in acetonitrile, the compounds were immobilized on a glassy carbon electrode using a slurry-casting method. The slurry for each sample was prepared using a 50:40:10 ratio of COE-CbzBP (or congener), carbon black, and polytetrafluoroethylene (PTFE), respectively. Tetrabutylammonium hexafluorophosphate (TBAPF₆) was used as the supporting electrolyte.

Rehm-Weller Equation—The thermodynamic feasibility of photoinduced electron transfer between donor (D) and acceptor (A) moieties can be estimated using the Rehm-Weller equation, which calculates the Gibbs free energy change (ΔG_{CS}) associated with the formation of a radical ion pair in the excited state. The equation is given by:

$$Δ {G}_{CS} = {N}_{\text{?}} e ( {E}_{ox} - {E}_{red} ) + \frac{{e}^{2}}{4 π {\text{?}}_{0}} ( ( \frac{1}{2 {r}_{1}} + \frac{1}{2 {r}_{2}} ) ( \frac{1}{{\text{?}}_{\text{?}}} - \frac{\text{?}}{{\text{?}}_{\text{?}}} ) - \frac{1}{{τ}_{\text{?}} {\text{?}}_{\text{?}}} ) - {E}_{\text{?}}$$

$$\text{?} \text{indicates text missing or illegible when filed}$$

where N_{A} is Avogadro's number, e is the elementary charge, Eₒₓ and E_{red} are the oxidation and reduction potentials, respectively, E₀, is the vacuum permittivity, r₁ (~5 Å) and r₂ (~5 Å) are the effective ionic radii of the donor and acceptor respectively, Eₒₓ and E_{red} are the dielectric constant of the solvent, r₁₂ is the donor-Acceptor distance (10 Å) and the dielectric constant of the microevent (Water ~80, DNA ~8, and liposome ~4). This equation captures both the redox driving force and the electrostatic stabilization (or destabilization) of the ion pair, accounting for solvent and molecular geometry effects. A negative ΔG_{CS} indicates that the formation of the spin-correlated radical pair is thermodynamically favorable. The Rehm-Weller formalism was applied to rationalize the quantum entangled spin-pair generation observed in the COE-CbzBP system under biologically relevant conditions.

### Example 1

### Synthesis of HCTO-BPC (5) and ICTO-BPC (COE-CbzBP)

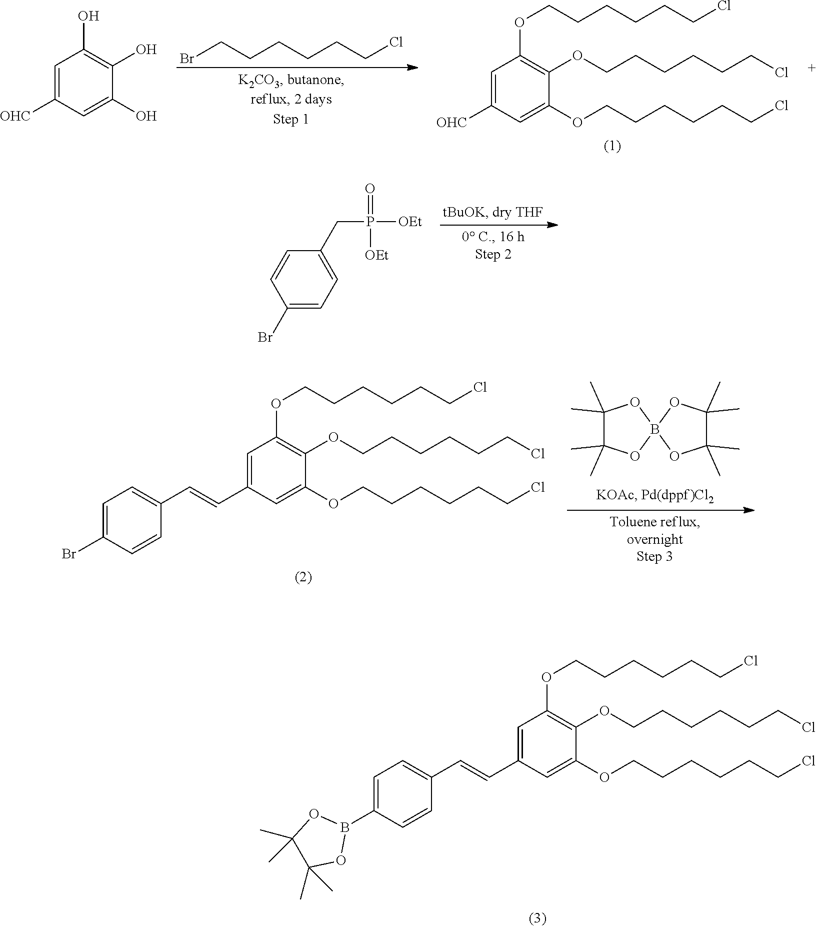

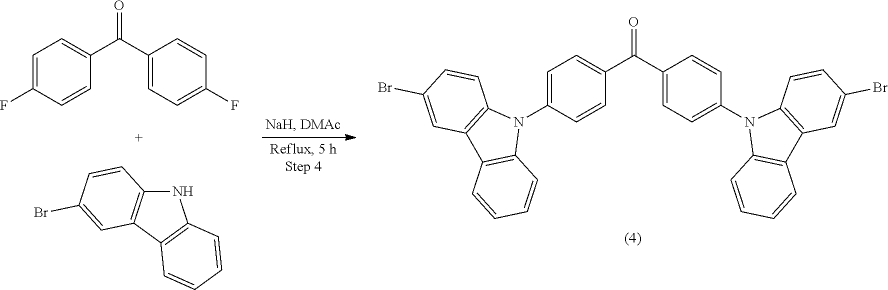 [SMILES: O=C(c1ccc(F)cc1)c1ccc(F)cc1 ; Brc1ccc2[nH]c3ccccc3c2c1 ; O=C(c1ccc(-n2c3ccccc3c3cc(Br)ccc32)cc1)c1ccc(-n2c3ccccc3c3cc(Br)ccc32)cc1]

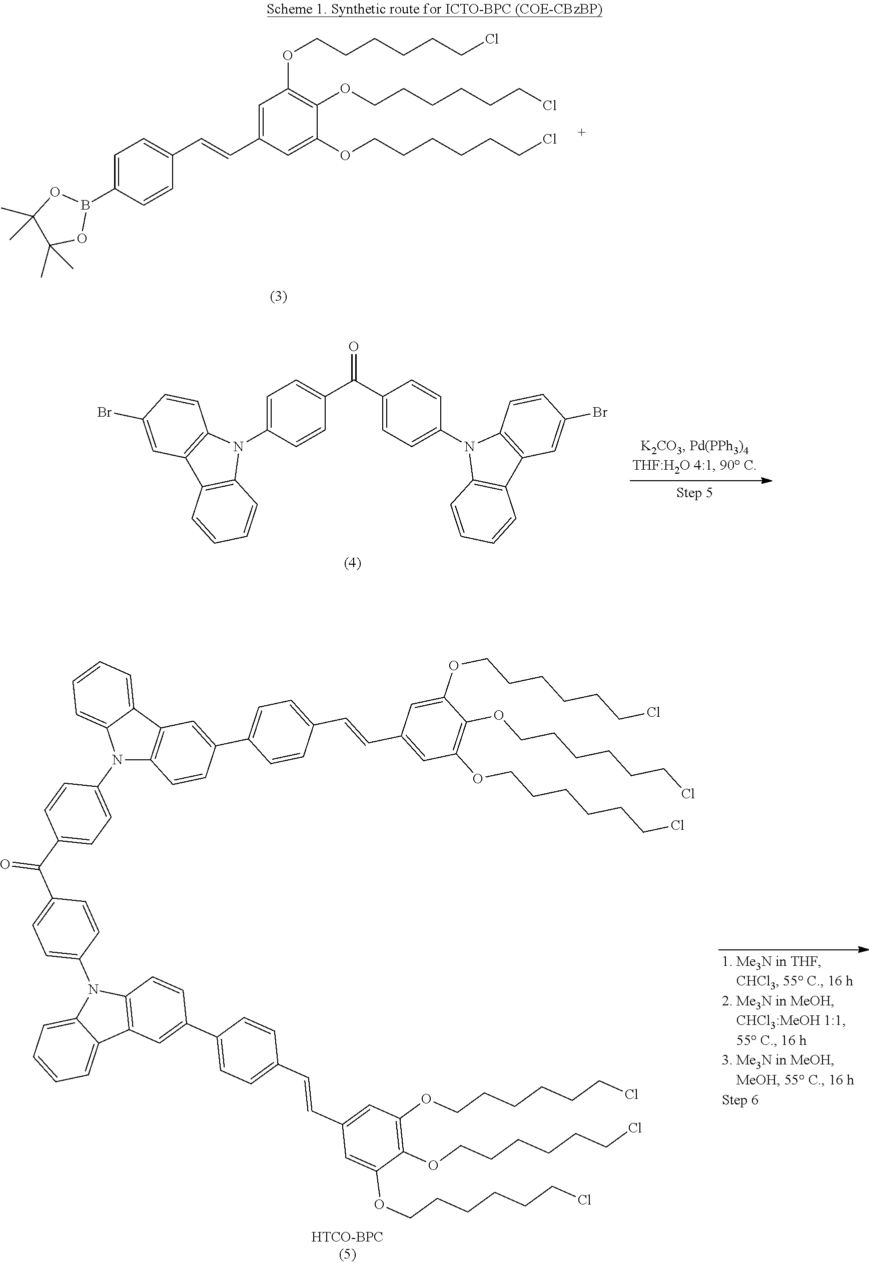 [SMILES: CC1(C)OB(c2ccc(/C=C/c3cc(OCCCCCCCl)c(OCCCCCCCl)c(OCCCCCCCl)c3)cc2)OC1(C)C ; O=C(c1ccc(-n2c3ccccc3c3cc(Br)ccc32)cc1)c1ccc(-n2c3ccccc3c3cc(Br)ccc32)cc1 ; O=C(c1ccc(-n2c3ccccc3c3cc(-c4ccc(/C=C/c5cc(OCCCCCCCl)c(OCCCCCCCl)c(OCCCCCCCl)c5)cc4)ccc32)cc1)c1ccc(-n2c3ccccc3c3cc(-c4ccc(/C=C/c5cc(OCCCCCCCl)c(OCCCCCCCl)c(OCCCCCCCl)c5)cc4)ccc32)cc1]

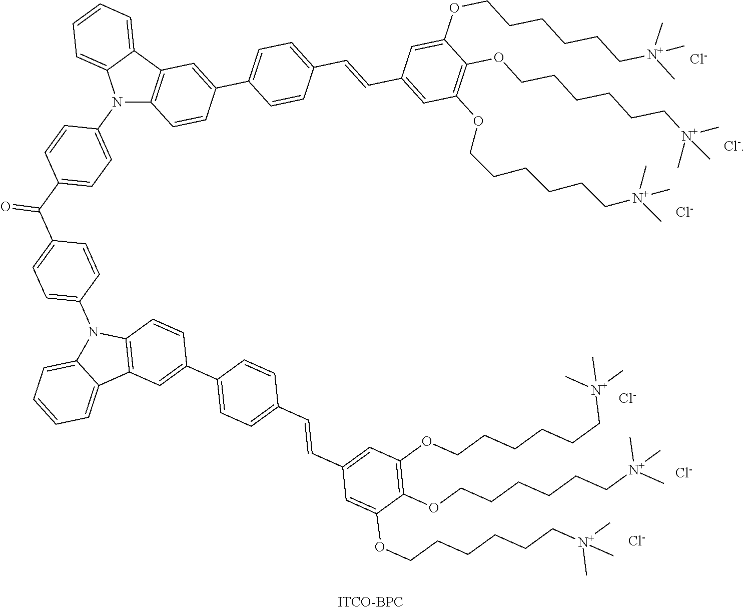

Step 1: In a dried round bottomed flask, 3,4,5-Trihydroxybenzaldehyde (1 equiv.) was dissolved in butanone (3 mL) under inert atmosphere. To this solution, potassium carbonate (6 equiv.) was added and stirred for 15 minutes at room temperature. 1-Bromo-6-chlorohexane (6 equiv.) was then added to this resultant mixture and stirred for another 15 minutes at room temperature. Finally, the reaction mixture was refluxed for 2 days. Once the reaction was complete, the resultant mixture was subjected to a rotary evaporator and was concentrated. The concentrated mixture was then extracted with dichloromethane and doubly deionized (DI) water. The organic layer was collected and dried over sodium sulfate. The solvent obtained after extraction was then concentrated using rotary evaporator to obtain a brownish liquid compound and was further purified using silica gel column chromatography using a mixture of hexane and ethyl acetate as eluent to obtain a pale-yellow liquid as the pure compound. The pure compound (1) was then characterized using ¹H-NMR and ¹³C-NMR spectroscopy.

¹H NMR (500 MHz, CDCl₃): δ 9.83 (s, 1H), 7.08 (s, 2H), 4.07-4.03 (m, 6H), 3.56-3.53 (m, 6H), 1.88-1.74 (m, 12H), 1.57-1.48 (m, 12H).

¹³C NMR (125 MHz, CDCl₃): δ 191.32, 153.55, 143.76, 131.70, 107.99, 73.46, 69.11, 45.16, 45.07, 32.74, 32.64, 30.28, 29.23, 26.85, 26.72, 25.53, 25.48.

Step 2: Potassium tert-butoxide (3 equiv.) in dry THF (2 mL) was taken in a dried sealed tube and stirred under argon atmosphere in ice-cold condition. To this solution Diethyl (4-Bromobenzyl) phosphonate (4 equiv.) was added and allowed to stir for 2-3 minutes. Following this, (1) dissolved in dry THF (2 mL) was added to the mixture and stirred for 15 minutes. After this, the resultant mixture was left for stirring for 16 hours at room temperature. The crude mixture obtained after completion of the reaction was extracted with dichloromethane and DI water. The organic layer was collected, dried over sodium sulfate and concentrated using a rotary evaporator. The pale-yellow liquid obtained was then purified using a silica gel column chromatography using mixture of ethyl acetate and hexane as eluent to yield a colourless liquid compound (2) as pure product. The product was then characterized using ¹H-NMR and ¹³C-NMR spectroscopy.

¹H NMR (500 MHz, CDCl₃): δ 7.47 (d, J=8.5 Hz, 2H), 7.35 (d, J=8.6 Hz, 2H), 6.99 (d, J=16.2 Hz, 1H), 6.90 (d, J=16.2 Hz, 1H), 4.03 (t, J=6.4 Hz, 4H), 3.97 (t, J=6.4 Hz, 2H), 3.57-3.54 (m, 6H), 1.87-1.74 (m, 12H), 1.57-1.49 (m, 12H).

¹³C NMR (125 MHz, CDCl₃): δ 153.36, 138.48, 136.42, 132.49, 131.92, 129.62, 127.98, 126.74, 121.30, 105.43, 73.38, 69.07, 45.22, 45.13, 32.80, 32.69, 30.29, 29.42, 26.94, 26.77, 25.59.

Step 3: In a round bottom flask under argon atmosphere, (2) (1 equiv.), Bis(Pinacolato)Diboron (2 equiv), potassium acetate (3 equiv.) and Pd(dppf)Cl₂ (0.05 equiv.) was added in dry toluene (5 mL) under argon atmosphere and the reaction mixture was left for stirring overnight at reflux. Once the reaction was complete, the mixture was extracted with dichloromethane and DI water and the collected organic layer was passed through sodium sulfate to dry any remaining water. The solution was then concentrated using a rotary evaporator and was purified using silica gel chromatography with different ratios of hexane and ethyl acetate as the eluent. The pure compound (3) was obtained as white solid which was then confirmed using ¹H-NMR and ¹³C-NMR spectroscopy.

¹H NMR (500 MHz, CDCl₃): δ 7.79 (d, J=8.1 Hz, 2H), 7.49 (d, J=8.0 Hz, 2H), 7.07 (d, J=16.2 Hz, 2H), 6.98 (d, J=16.2 Hz, 1H), 6.72 (s, 2H), 4.03 (t, J=6.4 Hz, 4H), 3.97 (t, J=6.4 Hz, 2H), 3.57-3.54 (m, 6H), 1.86-1.75 (m, 7H), 1.55-1.49 (m, 12H), 1.35 (s, 12H).

¹³C NMR (125 MHz, CDCl₃): δ 153.33, 140.14, 138.37, 135.31, 132.72, 129.83, 127.96, 125.82, 105.41, 83.94, 73.38, 69.03, 45.24, 45.15, 32.81, 32.71, 30.30, 29.43, 26.95, 26.79, 25.61, 25.03.

Step 4: 3-Bromocarbazole (3 equiv.) dissolved in 4 mL dry N,N-Dimethylacetamide (DMAc) was added in a double-neck round bottom flask under inert atmosphere. To this solution, sodium hydride (2.5 equiv.) was added slowly in ice-cold condition and was left to stir for 30 minutes. After 30 minutes, 4,4′-Difluorobenzophenone (1 equiv.) dissolved in 1 mL dry DMAc was added dropwise using a needle and was heated to reflux for approximately 5 hours. Once the reaction was stopped, it was quenched by pouring it over ice-water. The precipitate obtained was filtered and washed with acetone multiple times to yield a pale yellow solid as pure product. The final compound (4) was characterized using ¹H-NMR and ¹³C-NMR spectroscopy.

¹H NMR (500 MHz, CDCl₃): δ 8.40 (s, 2H), 8.24-8.19 (m, 6H), 7.84 (d, J=8.5 Hz, 4H), 7.75-7.73 (m, 6H), 7.64-7.59 (m, 8H), 7.51-7.48 (m, 2H), 7.39-7.36 (m, 1H), 7.09 (d, J=16.2 Hz, 2H), 7.05 (d, J=16.2 Hz, 2H), 6.76 (s, 4H), 4.06 (t, J=6.4 Hz, 8H), 3.99 (t, J=6.4 Hz, 4H), 3.59-3.55 (m, 12H), 1.89-1.76 (m, 24H), 1.59-1.50 (m, 24H)

¹³C NMR (125 MHz, CDCl₃): δ 194.54, 153.35, 141.95, 140.85, 140.84, 139.86, 138.23, 135.99, 133.79, 132.94, 132.07, 128.68, 127.62, 127.59, 127.05, 126.65, 126.48, 125.64, 124.63, 124.13, 121.03, 120.73, 118.85, 110.26, 110.12, 105.34, 73.39, 69.05, 45.24, 45.15, 32.80, 32.70, 30.30, 29.44, 26.94, 26.79, 25.60.

Step 5: Compounds (3) (2.3 equiv.) and (4) (1 equiv.) obtained from the previous steps were taken in a round bottom flask under argon atmosphere. To this, potassium carbonate (6 equiv.) and Tetrakis(triphenylphosphine)palladium (0.05 equiv.) was added. Tetrahydrofuran (THF) and water in 4:1 ratio was added to the mixture which was then heated to 90° C. with constant stirring overnight. Once the reaction was stopped the crude reaction mixture was extracted using dichloromethane and DI water and the organic layer was collected over sodium sulfate. The collected fraction was concentrated and further subjected to column chromatography using different ratios of hexane and ethyl acetate as eluent. Yellowish solid product (5) obtained from column was then characterized using ¹H-NMR and ¹³C-NMR spectroscopy. Note that this compound, which is HCTO-BPC, exhibits multi-level quantum character.

¹H NMR (500 MHz, CDCl₃): δ 8.28 (d, J=1.9 Hz, 2H), 8.18 (d, J=8.5 Hz, 4H), 8.13-8.11 (m, 2H), 7.77 (d, J=8.4 Hz, 4H), 7.55-7.53 (m, 4H), 7.53-7.47 (m, 2H), 7.42 (d, J=8.7 Hz, 2H), 7.37-7.34 (m, 2H).

¹³C NMR (125 MHz, CDCl₃): δ 194.41, 141.59, 140.74, 139.09, 136.20, 132.10, 129.09, 127.16, 126.60, 125.80, 123.45, 122.94, 121.23, 120.86, 113.60, 111.40, 110.14.

Step 6: In a dried, sealed tube, compound (5) (1 equiv.), which is also referred to as HCTO-BPC, was taken in dry dichloromethane (12 mL). 2 M Trimethylamine in THF (4 mL) was then added to this solution in the tube under ice-cold conditions and was left to stir for 1 hour. The mixture was then heated to 55° C. and kept stirring for 16 hours. After 16 hours, the reaction mixture was concentrated in rota evaporator and then redissolved in 1:1 ratio of dichloromethane and methanol (12 mL). To this, 3.2 M Trimethylamine in methanol (4 mL) was added in ice-cold conditions and the mixture was then heated to 55° C. and kept stirring for another 16 hours. Again after 16 hours, the reaction mixture was concentrated, followed by redissolving in methanol (12 mL) and addition of 3.2 M Trimethylamine (4 mL) in methanol. The resultant mixture was again heated to 55° C. with stirring for 16 hours. After stopping the reaction, the crude mixture was concentrated to obtain a sticky yellow compound. This compound was triturated and washed multiple times with diethyl ether until a bright yellow solid compound is obtained. The final compound (referred to as ICTO-BPC or COE-CbzBP) was characterized using ¹H-NMR and ¹³C-NMR spectroscopy.

¹H NMR (500 MHz, DMSO-d6): δ 8.70 (s, 2H), 8.43 (d, J=7.6 Hz, 2H), 8.23 (d, J=8.1 Hz, 4H), 7.97 (d, J=8.0 Hz, 4H), 7.88 (d, J=8.2 Hz, 6H), 7.73-7.63 (m, 8H), 7.54 (t, J=7.7 Hz, 2H), 7.42-7.38 (m, 2H), 7.29 (d, J=16.4 Hz, 2H), 7.25 (d, J=16.4 Hz, 2H), 6.96 (s, 4H), 4.06 (t, J=6.4 Hz, 8H), 3.91 (t, J=6.3 Hz, 4H), 3.37 (s, 12H), 3.09 (s, 54H), 1.81-1.68 (m, 24H), 1.54-1.33 (m, 24H)

### Example 2

### Photophysical Properties of HCTO-BPC (5)

HCTO-BPC (compound (5)) was subjected to various photophysical studies to understand the photophysical properties of the molecule. HCTO-BPC is a precursor of COE-CbzBP (ICTO-BPC) but is not water soluble. However, it also exhibits similar multi-level quantum phenomena. Steady-state absorption of HCTO-BPC in solvent with different polarity are shown in Table 1 and FIG. 2.

TABLE 1
Steady-state absorption properties of HCTO-
BPC compounds in different organic solvents.

| Solvents | At λ max | At 350 nm |
|---|---|---|
| Toluene | 73352.48 | 73282.194 |
| Chloroform | 103658.628 | 103576.708 |
| THF | 86590.0993 | 86580.4195 |
| DCM | 105887.794 | 105769.205 |

Minimal changes were obtained in the absorption maxima wavelength, but changes in the absorption coefficient were obtained depending on the solvent. Table 1 summarizes the absorption coefficient values.

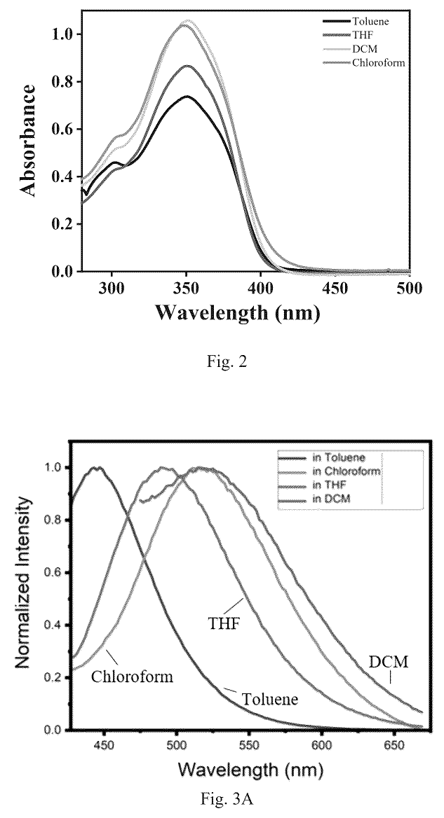

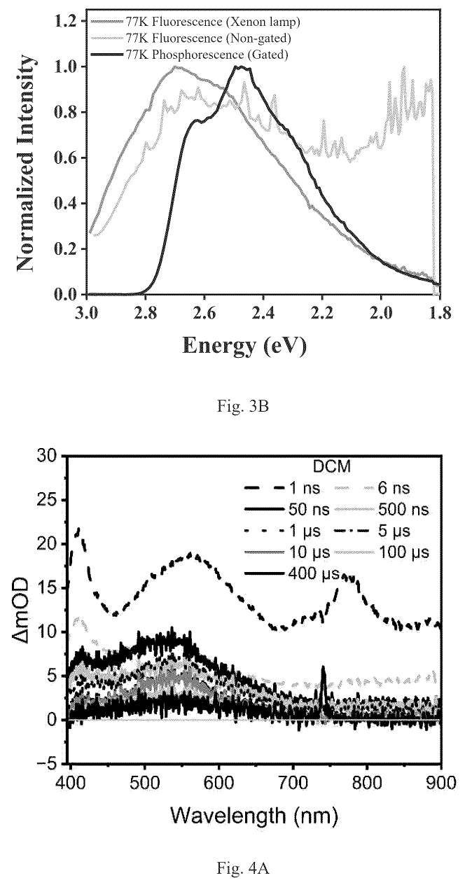

Steady-state emission of HCTO-BPC in solvents with different polarity are shown in FIGS. 3A-3B and Table 2. The emission wavelength maxima are more red-shifted with solvent polarity enhancement. Also, the quantum yield of HCTO-BPC is larger in solvents with lower polarities. Altogether, these results revealed that HCTO-BPC possesses strong charge-transfer characteristics. As FIG. 3B shows, HCTO-BPC also has overlapping singlet-triplet manifolds. Table 2 summarizes relevant photophysical properties such as quantum yield and the value for the singlet-triplet energy gap.

TABLE 2
Steady-state fluorescence properties of HCTO-BPC in different
organic solvents with steady-state gated emission properties
of HCTO-BPC to determine the singlet and triplet energy levels.

| | | | | | S₁ − T₁ |
|---|---|---|---|---|---|
| Solvents | RI | Slope | QY(%) | QY | Gap |
| Toluene | 1.496 | 588,499,546.21 | 34.80148 | 0.34805 | ~0.3 eV |
| Chloroform | 1.445 | 258,618,666.01 | 14.26869 | 0.142687 | ~0.3 eV |
| THF | 1.4072 | 894,125,577.54 | 37.5428 | 0.375428 | ~0.3 eV |
| DCM | 1.4244 | 58,438,206.15 | 2.51407 | 0.025141 | ~0.3 eV |

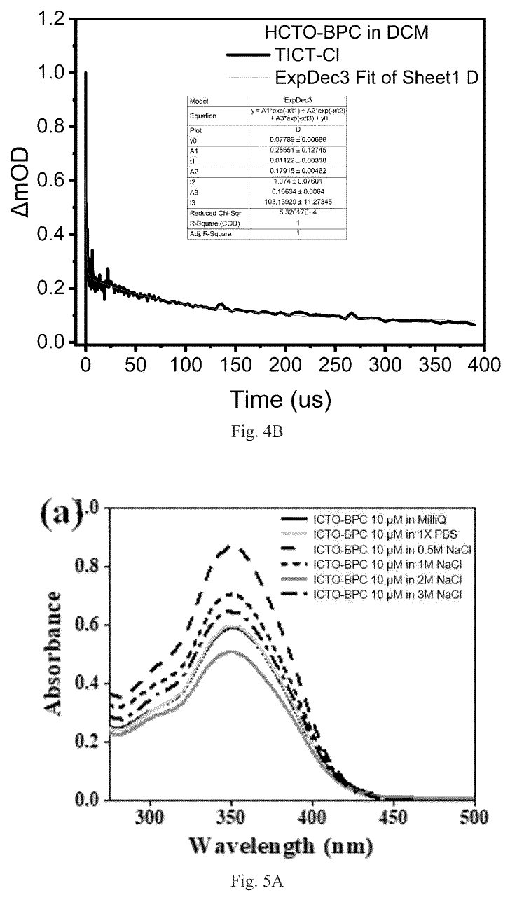

The transient absorption spectra of HCTO-BPC in DCM (FIG. 3A) revealed long-lived ESA up to 400 us. The ESA band tents towards 600 nm, indicating the presence of ketyl radicals, which have Dₙ character, see FIG. 4A. The transient absorption kinetic decay confirmed the multi-level quantum existence in HCTO-BPC, as a long-lived multicomponent quantum decay consistent with the simultaneous quantum coexistence of Tₙ and Dₙ was obtained at room temperature, FIG. 4B.

### Example 3A

### Molecular Design and Electronic Considerations

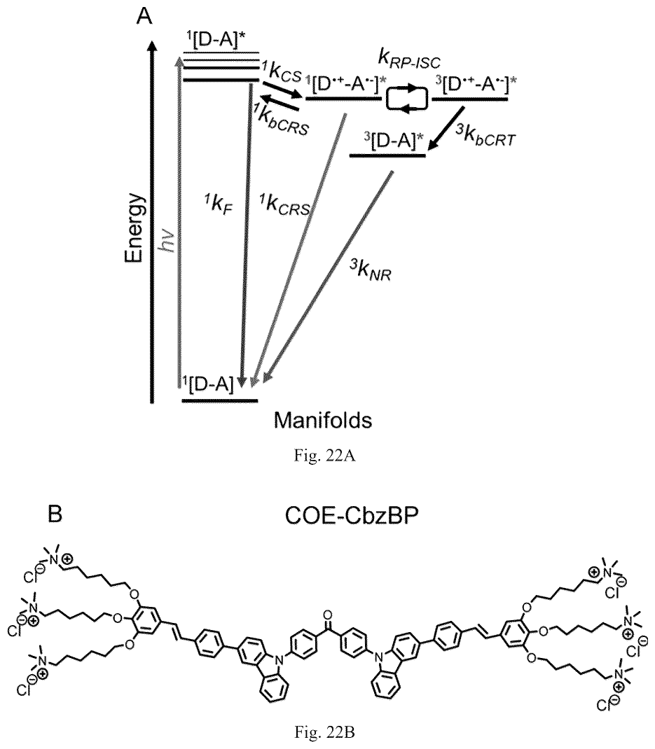

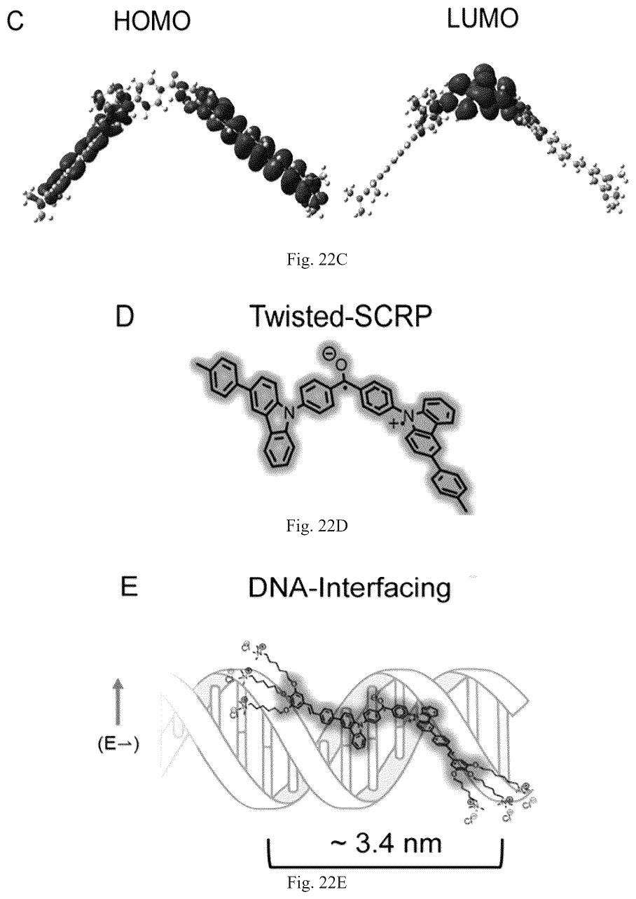

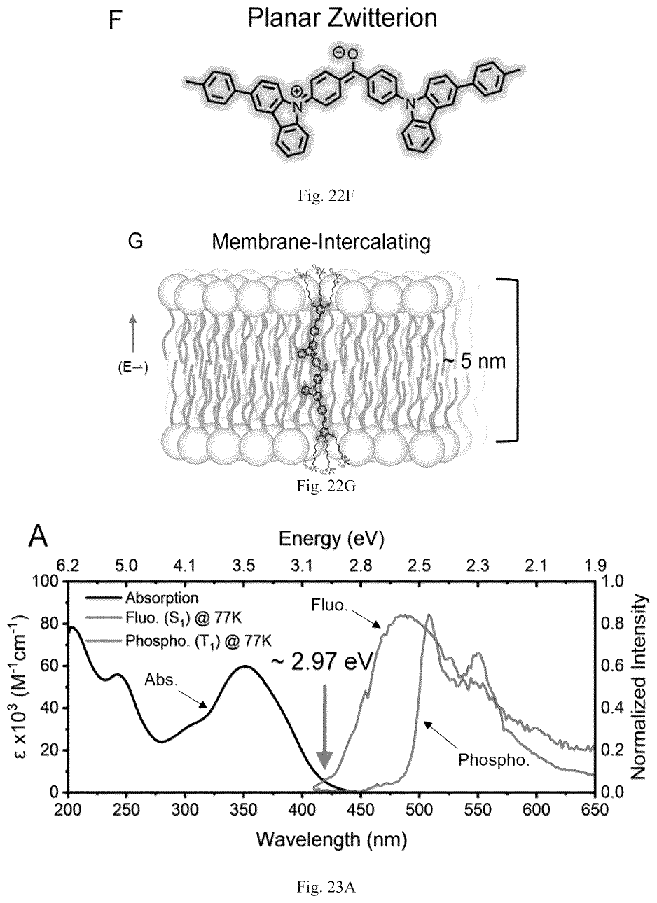

It has been established that introducing a partial break in 71-conjugation between an N-heteroarene and an electron-deficient acceptor, specifically at the N-C junction, one can promote spatial separation between the highest occupied molecular orbital (HOMO) and the lowest unoccupied molecular orbital (LUMO). This spatial orbital decoupling reduces their overlap and supports the formation of twisted intramolecular charge-transfer (TICT) states with minimized ΔE_{ST}. From a quantum mechanical standpoint, this HOMO-LUMO spatial separation minimizes both the Coulomb repulsion energy (J_{HL}) and the electron exchange energy (K_{HL}), with ΔE_{ST}=2K_{HL}, directly impacting singlet-triplet dynamics and spin correlation Consequently, it has been hypothesized that this strategy for minimizing K_{HL} is adaptable to SCRP systems based on Anderson's perturbation framework, where the K_{HL} correlates with J_{RP}, which modulates spin mixing and recombination yields in such systems. For further context, the ΔE_{ST} of a two-spin system correlates with J_{RP} when both share the same donor-acceptor orbital origin, as both reflect similar underlying orbital interactions. In this context, J_{RP} correlates with K_{HL}, following the relationship J_{RP}~ΔE_{ST}/2~K_{HL}. See Equations 1 and 2.

$$2 {J}_{R P} = Δ {E}_{S} - Δ {E}_{T} = \frac{{V}_{R P - S}^{2}}{Δ {E}_{S}} - \frac{{V}_{R P - T}^{2}}{Δ {E}_{T}} \qquad ( 1 )$$

$${J}_{R P} ∼ \frac{Δ {E}_{S T}}{2} ∼ {K}_{H L} \qquad ( 2 )$$

Where ΔE_{N} (ΔE_{S} and ΔE_{T}) expand to E_{RP}−E_{N}−λ_{T}, and each term represents the contributions from radical pairs, Frenkel exciton states, and reorganization energies, respectively, for each state. V_{RP-N} (V_{RP-S} and V_{RP-T}) is the electronic coupling between the singlet and triplet radical pairs and their neighboring states. For simplicity, it is proposed to treat ΔE_{S} and ΔE_{T} as the fluorescence and phosphorescence of the probe, respectively, ultimately equating K_{HL} and J_{RP}. The incorporation of carbazole and benzophenone with a partial break in the N-C junction yielded an electronic structure with a minimal HOMO-LUMO overlap, see FIG. 22C.

It is expected that the resulting COE-CbzBP emulates the electronic behavior of Michler's ketone, a symmetric TICT chromophore featuring dimethylamine donors instead of carbazoles. Michler's ketone is known to form photogenerated radical pairs in twisted geometries but exhibits zwitterionic charge-transfer character in its planar conformation. It was hypothesized that upon photoexcitation, a TICT-promoted cationic radical is formed primarily on the carbazole ([Cbz^{·+}]*) and an anionic radical formed primarily on the benzophenone ([BP^{·-}]*), generating a SCRP that can respond to electric fields, such as those present in DNA, see FIGS. 22D and 22E. Conversely, when intercalated into lipid bilayers, COE-CbzBP likely adopts a near-planar, zwitterionic conformation that suppresses the formation of SCRP, preventing electric field sensing (FIGS. 22F and 22G).

### Example 3B3

### Steady-State Electronic Spectroscopy for Photophysical Properties of ICTO-BPC (Also Referred to as COE-CbzBP)

ICTO-BPC was subjected to various photophysical studies to understand the photophysical properties of the molecule. Initially, the UV-vis technique was used to characterize the absorption properties of ICTO-BPC in various aqueous solutions. The results demonstrated a strong absorption band at ~351 nm attributed to the π-π* transitions and a weak absorption band at 303 nm recorded in the solutions (MilliQ, 1×PBS, 0.5 M NaCl, 1 M NaCl, 2M NaCl and 3 M NaCl) with varying dielectric constants (FIG. 5A). No changes with respect to the wavelength were noted for the absorption recorded in any of these solutions. However, the different absorption coefficients calculated in these solutions were indicative that the ground state characteristics of the ICTO-BPC in these solutions change based on the salt composition or the salt concentrations (FIG. 5A). The absorption coefficients calculated for ICTO-BPC in the respective solutions have been provided in Table 3.

TABLE 3
Photophysical properties of ICTO-BPC in different solutions.

| | Absorption | Absorption | Emission | |
|---|---|---|---|---|
| | Coefficient | Maxima | Maxima | |
| Solvents | (ε, M⁻¹cm⁻¹) | (λ_{abs}) | (λₑₘ) | |
| MilliQ | 59,236 | ~351 nm | ~528 | nm |
| MilliQ + DNA | N/A | ~351 nm | ~520 | nm |
| 1X PBS | 59,834 | ~351 nm | ~522 | nm |
| 1X PBS + DNA | N/A | ~351 nm | ~520 | nm |
| 0.5M NaCl | 86,700 | ~351 nm | ~521 | nm |
| 1M NaCl | 70,500 | ~351 nm | ~521.5 | nm |
| 2M NaCl | 50,622 | ~351 nm | ~522 | nm |
| 3M NaCl | 64,900 | ~351 nm | ~520 | nm |
| Liposome | n/a | n/a | ~460 | nm |
| 20% Glycerol | n/a | ~351 nm | ~500 | nm |
| 40% Glycerol | n/a | ~351 nm | ~500 | nm |
| 60% Glycerol | n/a | ~351 nm | ~495 | nm |
| Tris | n/a | ~351 nm | ~522 | nm |
| Tris + DNA | n/a | ~351 nm | ~520 | nm |

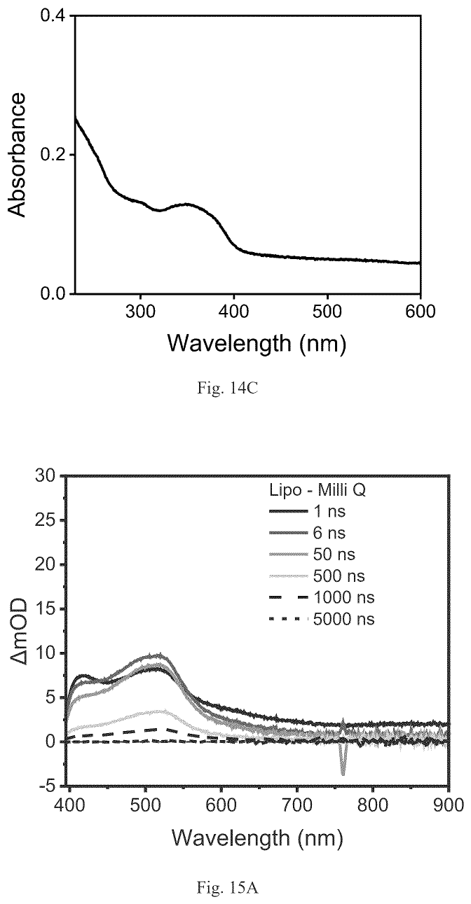

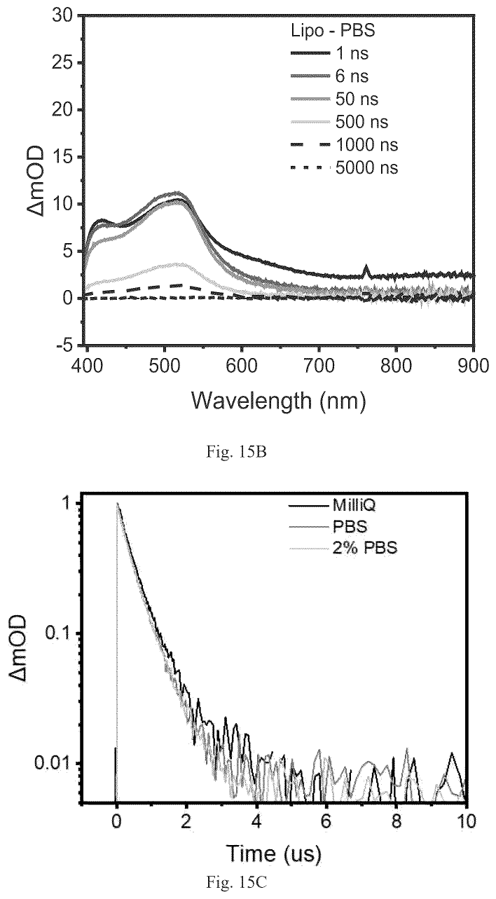

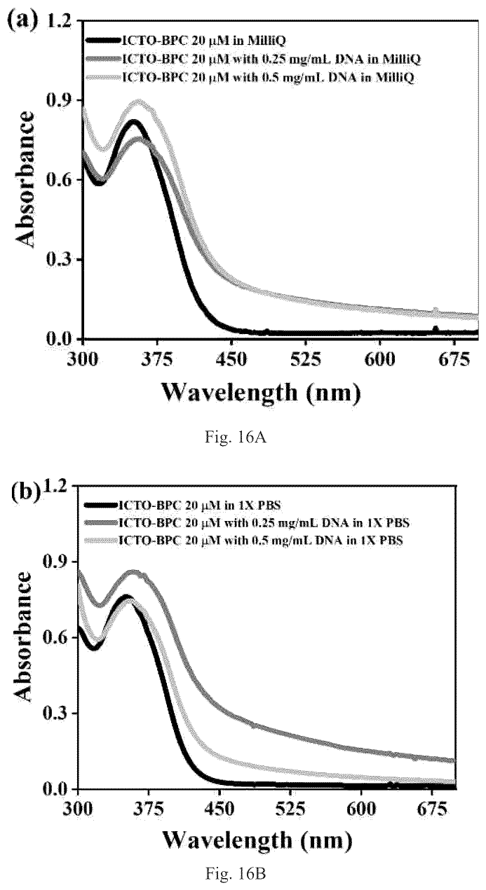

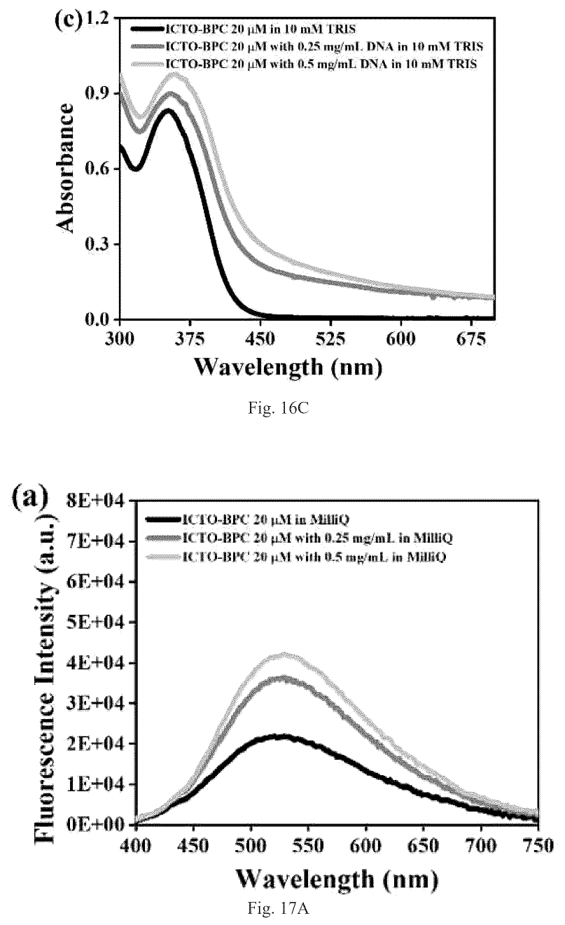

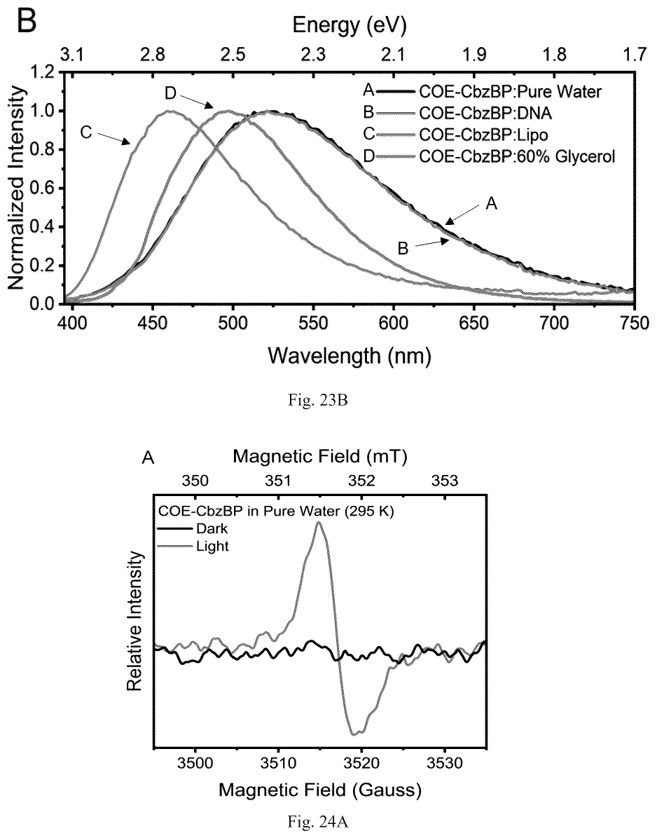

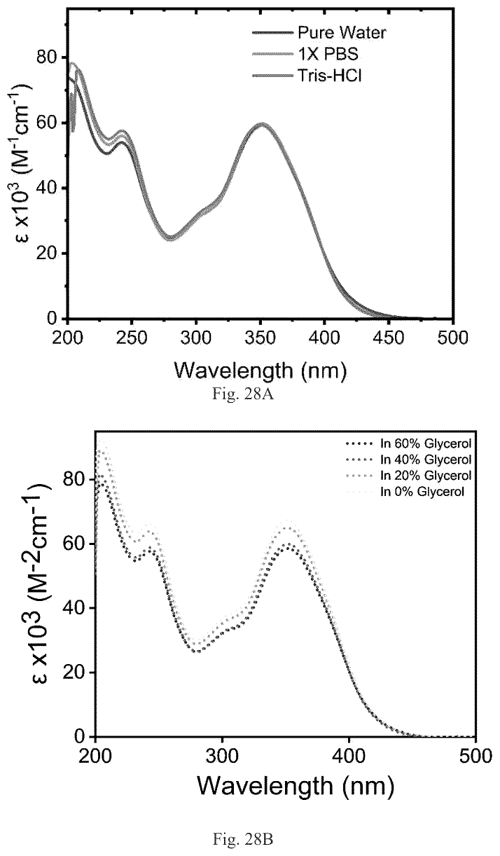

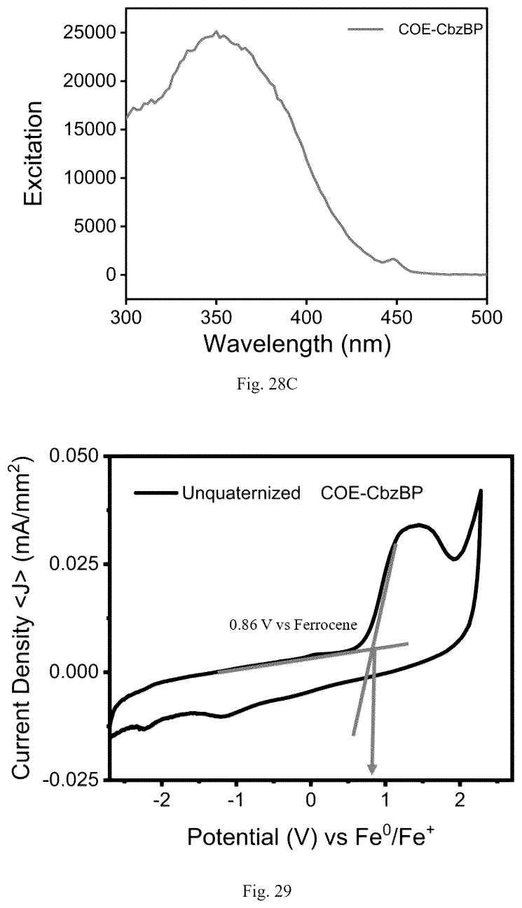

The absorption and emission spectra of COE-CbzBP were recorded in pure water at room temperature, see FIG. 23A. Quantitative spectroscopic data, including absorption maxima (λ_{AbsMax}), molar absorptivity coefficient (ε), and emission maxima (λ_{EmMax}) across different environments, are summarized in Table 4 and Table 5. In all cases, COE-CbzBP exhibited a high-energy band centered at wavelengths >275 nm, attributed to the localized excitation (π-π*; ε>10⁵ M⁻¹ cm⁻¹). A more prominent low-energy band, observed above 450 nm, is assigned to the charge-transfer transition (n-π*; ε>10⁵ M⁻¹ cm⁻¹), characteristic of donor-acceptor charge-transfer systems. Notably, the absorption profile remained consistent across all tested media, including biologically relevant environments such as 1×PBS and 10 mM Tris-HCl, demonstrating its spectral robustness, see FIGS. 28A-28C and Table 5. Note that UV-Vis spectral shifts upon DNA association were obtained, consistent with changes in the ground state dipole moment of the COE-CbzBP (Stark effects, FIG. 16).

Furthermore, to investigate the excited state properties of ICTO-BPC, steady-state fluorescence spectroscopic techniques were used. Upon excitation at 380 nm, the steady-state data displayed a remarkable emission response of ICTO-BPC characterized by an intense emission peak with maxima ranging between 520-528 nm in various solutions (FIG. 5B). It was observed that the fluorescence intensity of ICTO-BPC in the salt solutions demonstrated a significant increase in intensity compared to that recorded in MilliQ. The variation in the emission intensity of the compound suggested different characteristics of the excited state of the compound in the different solutions with varying salt composition and concentrations.

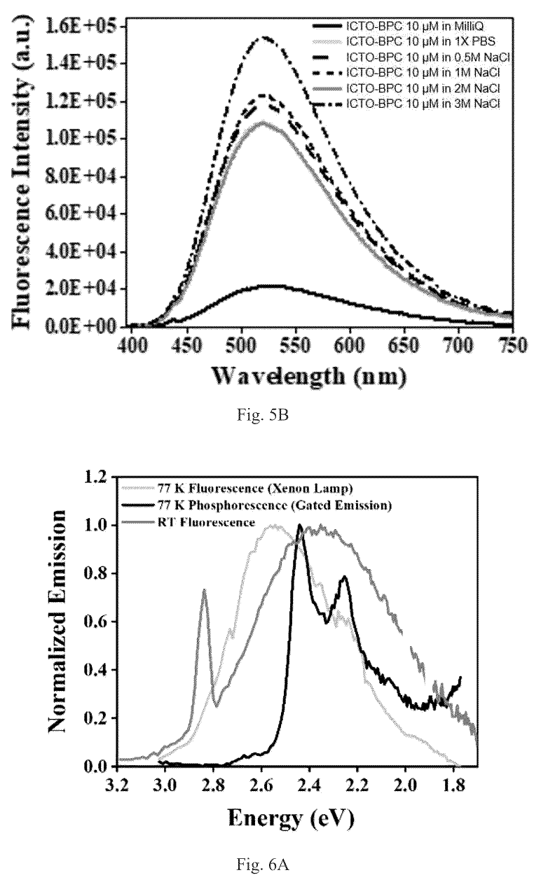

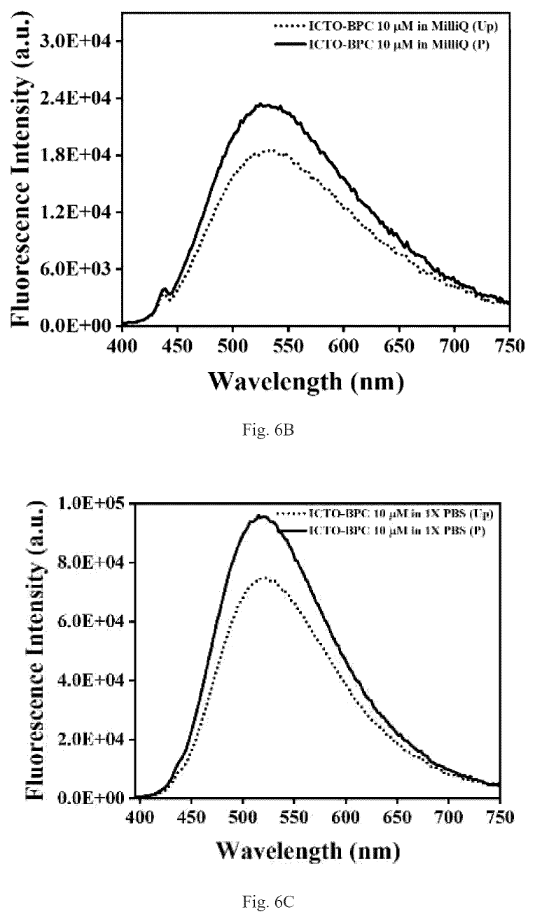

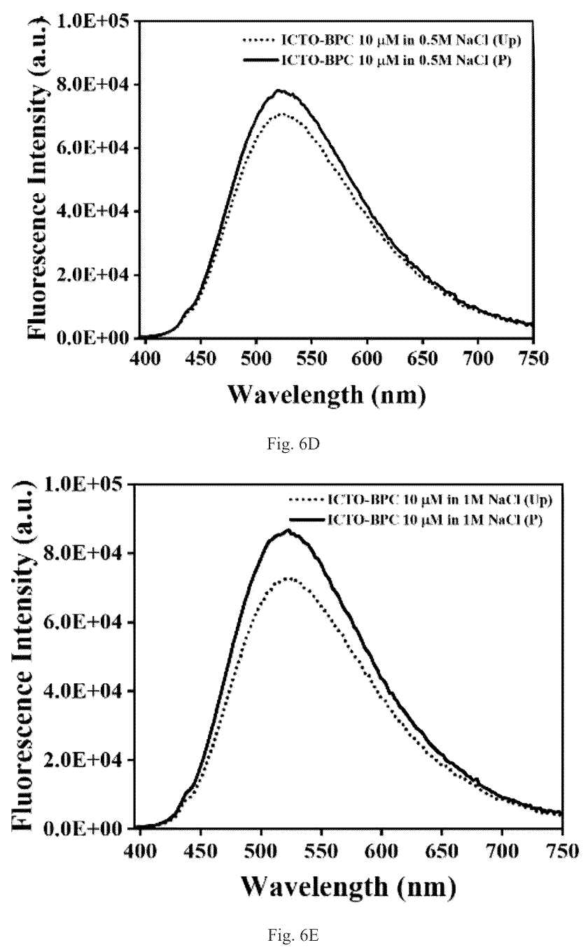

To obtain more insights into the excited state characteristics, more specifically the energies of the singlet and the triplet states of ICTO-BPC, phosphorescence measurement at 77K was conducted in solution (FIG. 6A). In this experiment, a multipulsed lamp of excitation 405 nm was used to excite the sample and the emission was detected after long delay to obtain the phosphorescence signal. The recorded data, when plotted with the emission response at 77 K, displayed overlapping fluorescence and phosphorescence spectra, exemplifying a minimal singlet (S1)-triplet (T1) energy gap and demonstrating overlapping quantum states. The minimal energy gap obtained between the S₁-T₁ states of ICTO-BPC indicates efficient singlet-triplet mixing and promotes thermally activated delayed fluorescence and multi-level quantum state characteristics. The emission response of ICTO-BPC was also monitored by purging the solution with argon to remove the dissolved oxygen levels in the solution, as oxygen is a known quencher of triplet and doublet states. Comparison of the emission properties of ICTO-BPC in each of the solutions in the presence and absence of oxygen have been demonstrated (FIGS. 6B-6F). The data indicated a noticeable increment in the emission intensities of the compound in each of the solutions when individually excited at 380 nm (FIGS. 6B-6F).

Determining the ΔE_{ST} of the COE-CbzBP experimentally was prioritized, which can be done using gated photoluminescence spectroscopy, see FIG. 23A. This technique allows spectral discrimination between fluorescence (S₁) and phosphorescence (T₁) of the material. Measurements were conducted under oxygen-free conditions at 77 K in pure water, using a pulsed 405 nm excitation source. The fluorescence spectra of COE-CbzBP exhibit an emission maximum at ~487 nm (~2.56 eV), while its phosphorescence spectrum exhibits an emission maximum at 508 nm (~2.44 eV). These values yield a ΔE_{ST} of 0.12 eV and a corresponding K_{HL} of 0.06 eV, which, based on Equation 2, gives a J_{RP} value of 0.06 eV. This experimental estimation of J_{RP} is only ~2.4× larger than k_{BT} (~25 meV), suggesting that thermally activated singlet-triplet manifold mixing might be possible, which is a highly desirable feature for spin-polarizable SCRP formation at room temperature. This becomes more apparent when considering the Rehm-Weller equation, which reveals that the radical pair formation step is positive but approaching thermoneutral in water (ΔG_{CS}=0.13 eV), yet becomes thermodynamically favorable in DNA (ΔG_{CS}=−0.19 eV) and liposomes (ΔG_{CS}=−0.55 eV). This thermodynamic profile suggests that electron transfer from the donor to the acceptor is feasible, enabling the formation of a quantum-entangled SCRP (see FIG. 29 for details on the Rehm-Weller calculation). Without wishing to be bound by any theory, this represents a water-soluble molecular probe exhibiting both the requisite electronic and electrochemical properties for SCRP formation. Given that the Rehm-Weller calculations revealed that the radical pair generation step seems to be thermodynamically favorable in the biological environments tested, COE-CbzBP offers a unique opportunity to investigate how TICT character influences SCRP formation and stability.

TABLE 4
Summary of the optical and electrochemical properties of COE-CbzBP.

| | λ_{AbsMax} | E_{g} | λ_{EmMax} | Φ | ΔG_{CS} | S₁ | T₁ | ΔE_{ST} | J_{RP} |
|---|---|---|---|---|---|---|---|---|---|
| Environment | (nm) | (eV) | (nm) | (%) | (eV) | (eV) | (eV) | (eV) | (eV) |
| COE-CbzBP | 351 | 3.1 | 522 nm | 3.3 | 0.13 | ~2.56 | ~2.44 | ~0.12 | 0.06 |
| COE- | 351 | 3.1 | 522 nm | 4.5 | −0.19 | ~2.38^{b} | n/a | n/a | n/a |
| CbzBP:DNA | | | | | | | | | |
| COE- | 351 | 3.1 | 461 nm | 8 | −0.55 | ~2.7^{b} | n/a | n/a | n/a |
| CbzBP:Lipo | | | | | | | | | |

Absorption maxima: λ_{AbsMax}; Bandgap (E_{g}); Emission maxima (λ_{EmMax}); Rehm-Weller calculation radical pair formation (ΔG_{CS}); fluorescence maxima (S₁); Phosphorescence maxima (T₁), Singlet-triplet energy splitting (ΔE_{ST}), electron exchange coupling (K_{HL}); and radical-pair exchange coupling (J_{RP}).
Note that the ΔE_{ST} was determined at 77K, where ice dominates the microenvironment of COE-CbzBP, constraining its geometry and preventing twisting.
Measurements at room temperature.^{b}

TABLE 5
The table summarizes the absorption coefficient values, and absorption
and emission maxima for COE-CbzBP recorded in free solution, in 60%
glycerol, in DNA, upon intercalation into liposomes, and in the
presence of DNA. K_{HL} was estimated with Equation
1 and 2 by assuming a V_{RP-N} average value of 0.1 eV

| | λ_{AbsMax} | E_{g} | λ_{EmMax} | S₁ | T₁ | ΔE_{ST} | |
|---|---|---|---|---|---|---|---|
| Environment | (nm) | (eV) | (nm) | (eV) | (eV) | (eV) | K_{HL}~J |
| Pure Water | 351 | 3.1 | 487 nm | 2.56 eV | 2.44 eV | 0.12 eV | 0.06 eV |
| (77K) | | | | | | | |
| Pure Water | 351 | 3.1 | 523 nm | 2.38 eV | 2.44 eV | −0.06 eV | −0.003 eV |
| (RT) | | | | | | | |
| Pure Water + DNA | 351 | 3.1 | 522 nm | 2.38 eV | | | |
| (RT) | | | | | | | |
| Tris | 351 | 3.1 | 522 nm | 2.38 eV | | | |
| (RT) | | | | | | | |
| Tris + DNA | 351 | 3.1 | 522 nm | 2.38 eV | | | |
| (RT) | | | | | | | |
| Liposome | 351 | 3.1 | 461 nm | 2.69 eV | | | |
| (RT) | | | | | | | |
| Glycerol | 351 | 3.1 | 500 nm | 2.48 eV | | | |
| (RT) | | | | | | | |

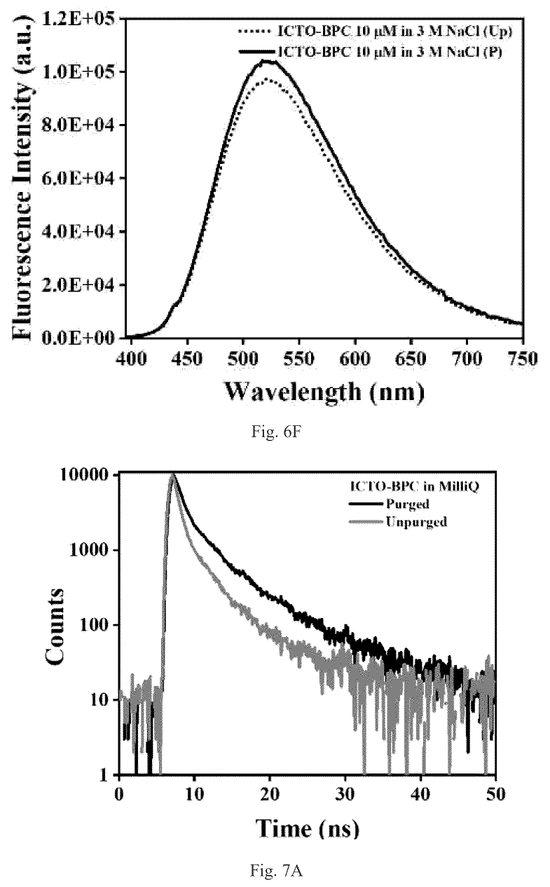

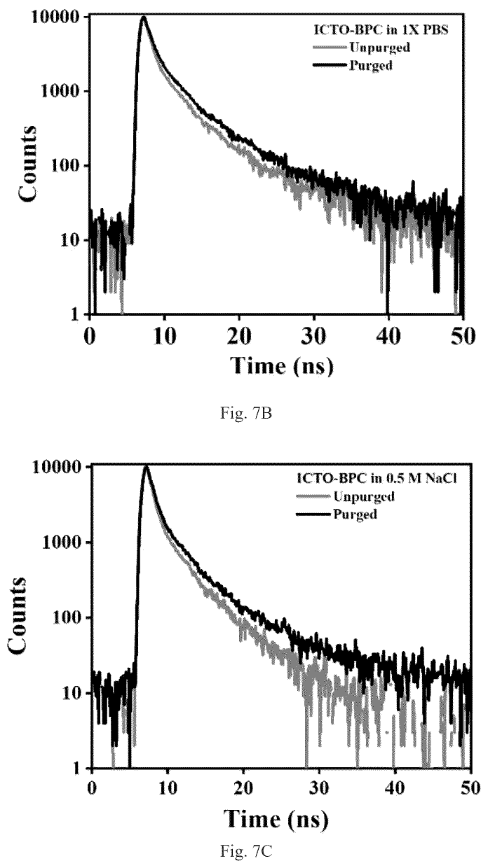

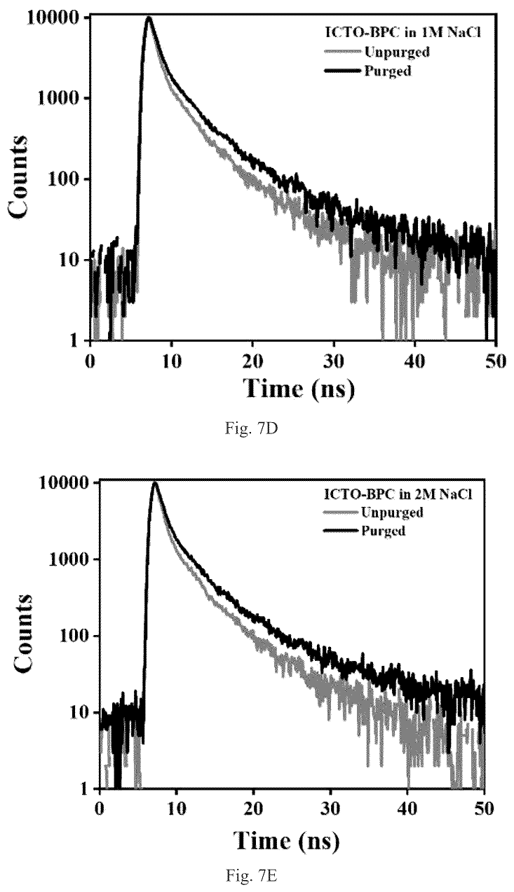

To further understand the excited state characteristics of ICTO-BPC, Time-correlated single-photon counting (TCSPC), an established time-resolved fluorescence spectroscopic technique for measuring fluorescence lifetimes of emissive materials was used. The fluorescence lifetime measurements were conducted for ICTO-BPC in various solutions both in the presence and absence of oxygen (FIGS. 7A-7F). The lifetime data of ICTO-BPC demonstrated bi-exponential decay in all the recorded solvents with lifetime (c) values ranging between 0.81 ns to 0.89 ns. Although, the lifetime of the compound in the absence of dissolved oxygen increased evidently, with calculated lifetime (c) values remaining around 1 ns. The shorter fluorescence lifetime values obtained here were attributed to the transfer of the singlet excited state photons into the triplet state owing to the lower S₁-T₁ energy barrier, which further corroborated the findings in the phosphorescence measurements of the compound at 77 K.

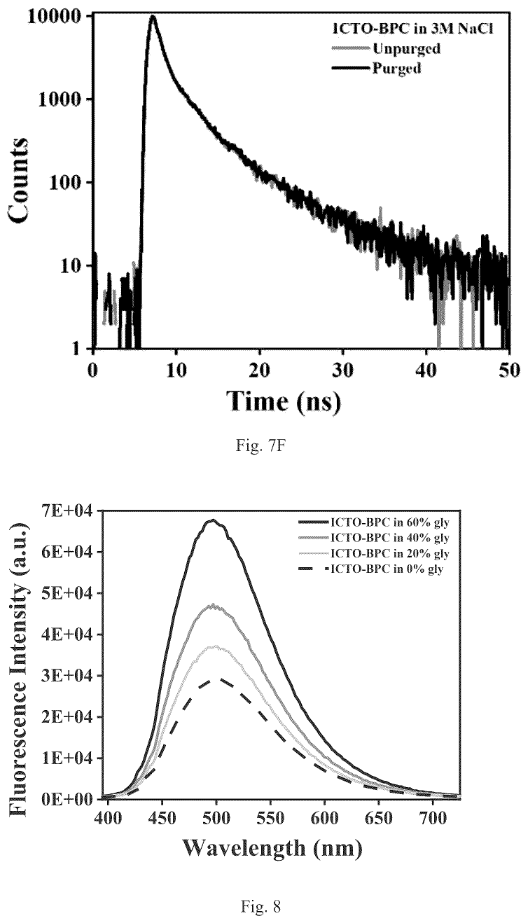

ICTO-BPC is also responsive to the microenvironment of the medium, for example, towards viscosity. This microenvironment responsiveness of ICTO-BPC is attributed to the twisted intramolecular charge transfer (TICT) design, which was validated by the glycerol-water experiment. Increasing the percentage of glycerol from 0 to 60% contributed to an enhancement in the emission intensity alongside a degree of hypsochromic shift in ICTO-BPC (FIG. 8). This was accredited to the enhanced viscosity of the medium, which resulted in minimizing rotational freedom across the single bonds in the molecule, accentuating the microenvironment responsiveness of the developed ICTO-BPC.

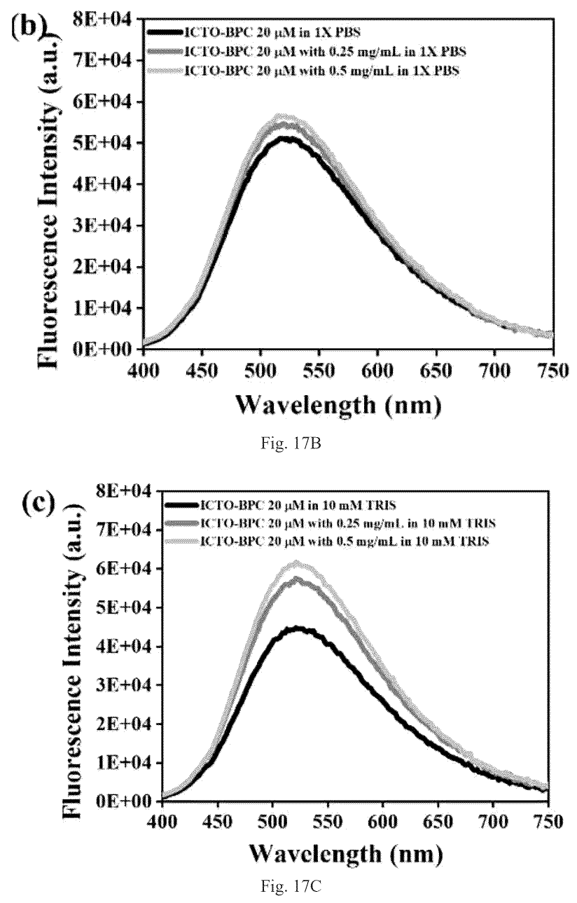

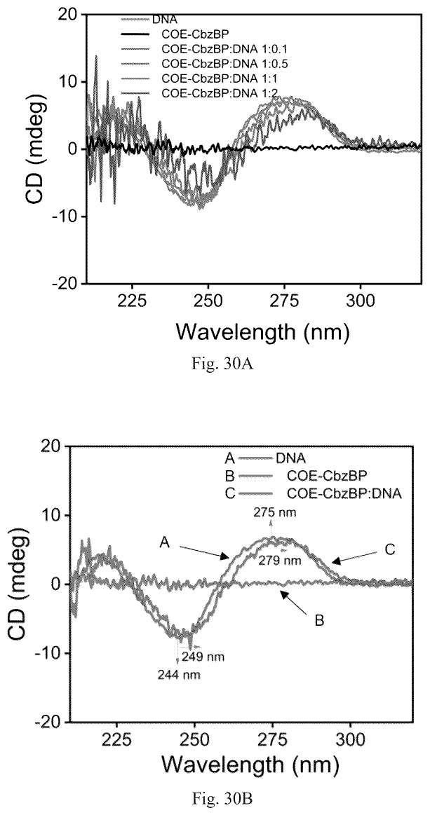

Long-lived radical pairs ideally exhibit low emissive quantum yields (Φ), as radiative recombination channels are a competing mechanism compromising possible qubit behavior. Accordingly, the emissive properties of COE-CbzBP was evaluated in aqueous and biologically relevant environments, see FIG. 23B. In high-dielectric media such as water, COE-CbzBP exhibited a red-shifted emission maximum (~522 nm), which remained unchanged upon association with DNA, indicating negligible solvatochromic effects. Circular dichroism spectroscopy confirmed the COE-CbzBP:DNA association, see FIGS. 30A and 30B. The (was approximately 3% in Tris-HCl buffer, meeting the criterion for low-emissive qubit-like platforms and suggesting minimal alteration of its radiative recombination channels upon association with DNA, see FIGS. 17A-17C and Table 4. In contrast, COE-CbzBP intercalated into lipid bilayers exhibited a blue-shifted emission (λ_{EmMax}=461 nm) and Φ values of 8%, consistent with localized excitations due to residing in a more rigid, less polar microenvironment (FIG. 23B). Measurements in glycerol solutions also yielded blue-shifted profiles (λ_{EmMax}=500 nm) but Φ comparable to aqueous environments (FIGS. 17A-17C and Table 6). These findings reinforce the TICT nature of COE-CbzBP and support its candidacy as a low-emissive, long-lived quantum probe based on radical pairs for biological settings.

TABLE 6
Table summarizing the quantum yield of the
COE-CbzBP in different environments.

| Sample | Quantum Yield |
|---|---|
| COE-CBZBP in MQ | 0.0175 |
| COE-CBZBP in 1X PBS | 0.0374 |
| COE-CBZBP in TRIS | 0.0334 |
| COE-CBZBP in 20%, 40% and 60% | 0.0221, 0.0279 and |
| GLY (Main solvent MQ) | 0.0396, respectively |
| COE-CBZBP with DNA in MQ | 0.0323 |
| COE-CBZBP with DNA in 1X PBS | 0.0416 |
| COE-CBZBP with DNA in TRIS | 0.0453 |
| COE-CBZBP in Liposomes | 0.08 |

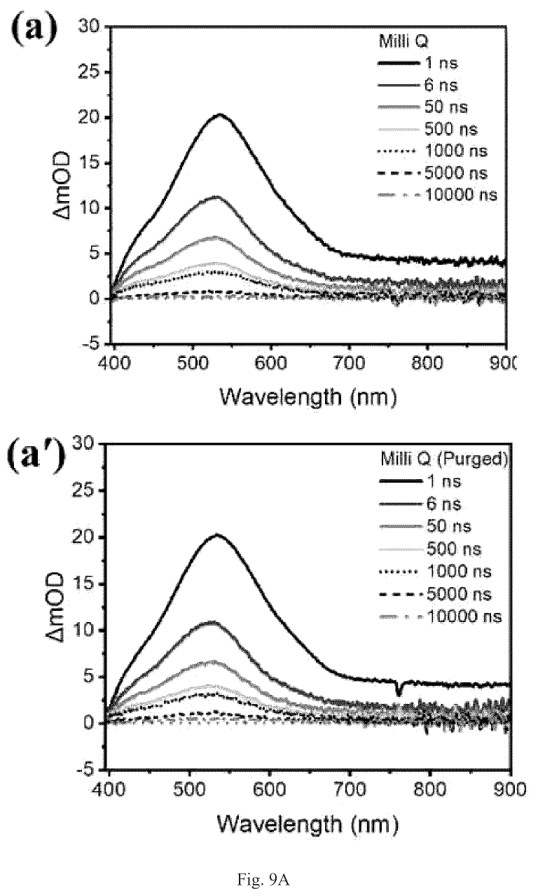

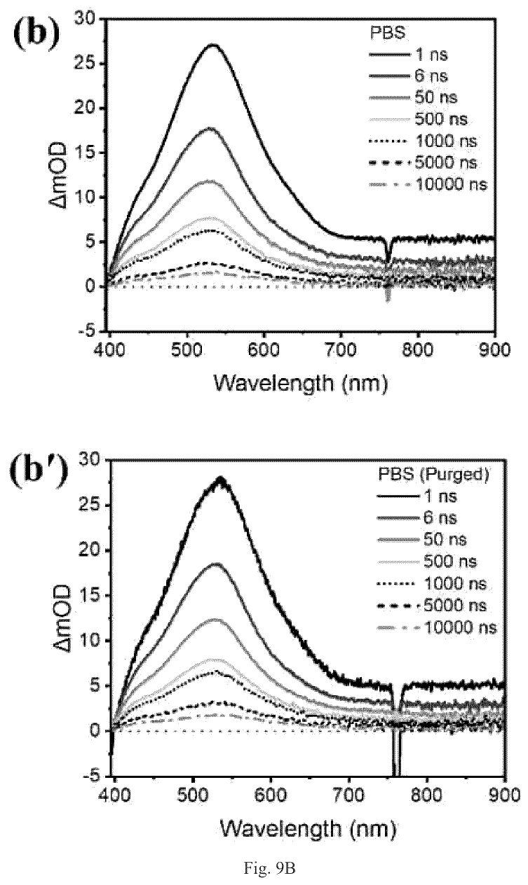

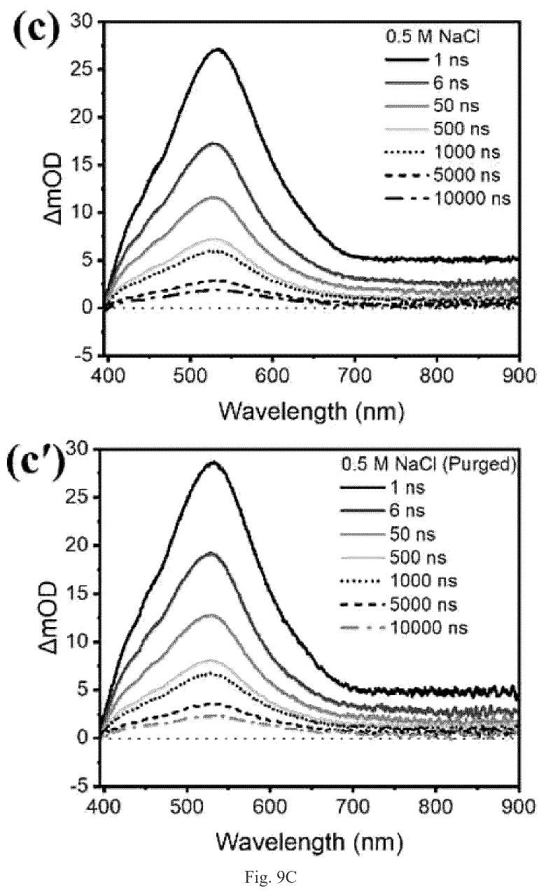

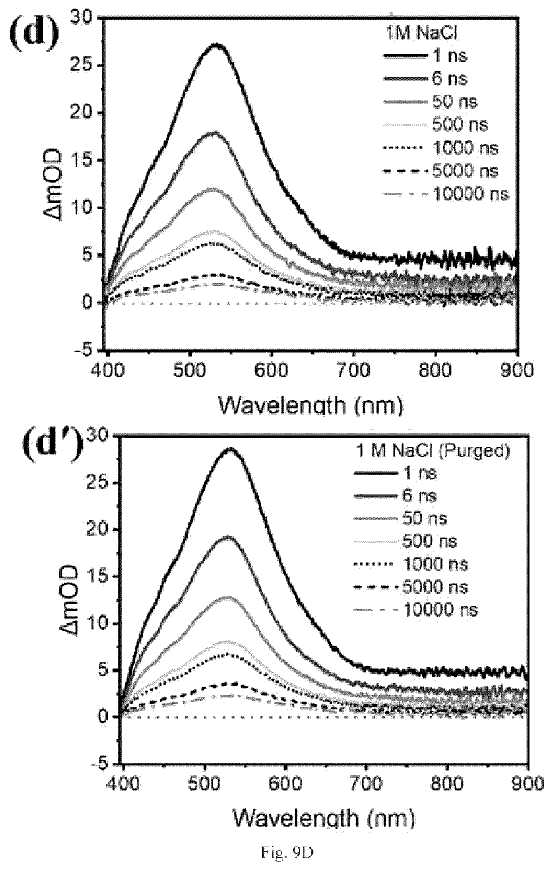

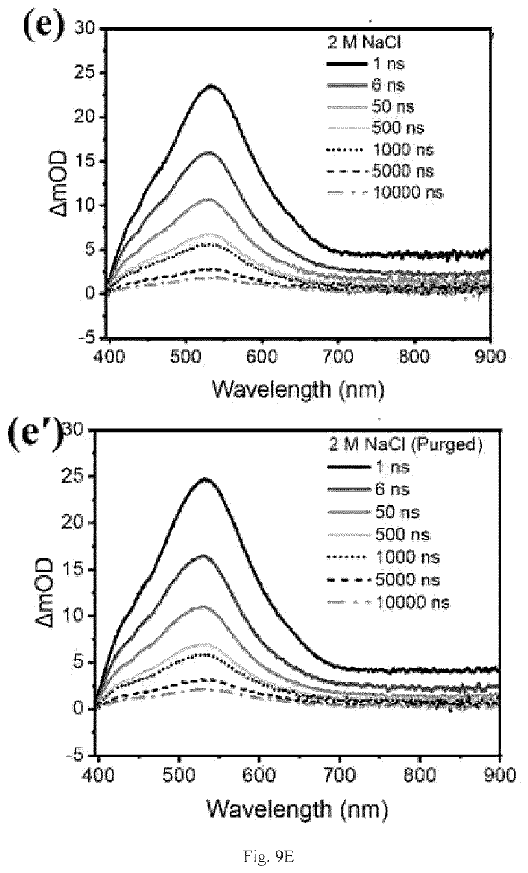

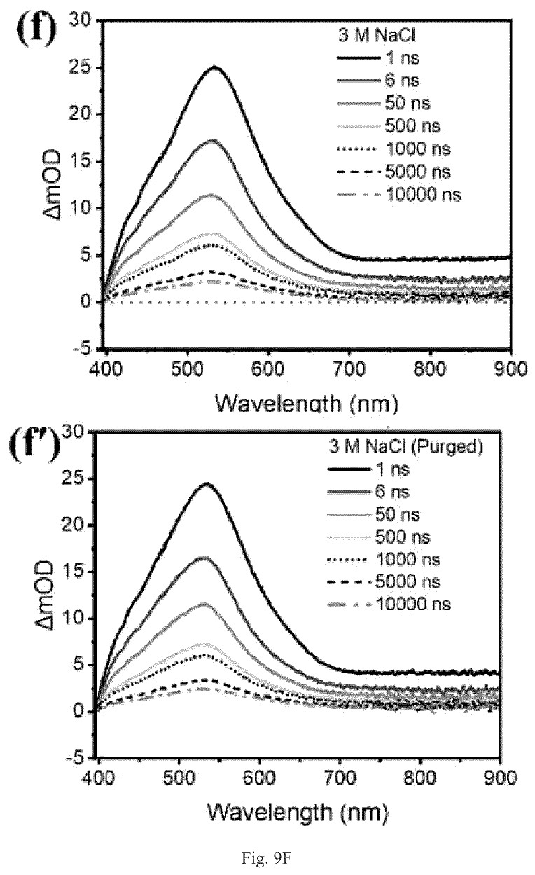

After obtaining sufficient information about the ground state and the emissive properties of ICTO-BPC, an in-depth investigation of the quantum state properties of the ICTO-BPC in various solvent systems using transient absorption spectroscopy. The data obtained from the transient measurements unveiled very intriguing results. The positive, excited state absorption (ESA) transient signal obtained for ICTO-BPC in all the solvents demonstrates the long-lived transient species associated with Tₙ or Dₙ quantum states (FIGS. 9A-9F), primarily promoted by the overlapping singlet-triplet quantum states as a precursor (FIG. 6A). That a sizable ESA was obtained up to at least 10 μs, the timescale provides information about the presence and decay profile of the long-lived quantum states. The data is conducive adequately to highlight that the quantum states assessed in the ICTO-BPC in all the tested solutions are sufficiently long-lived, well within the ρs timescale.

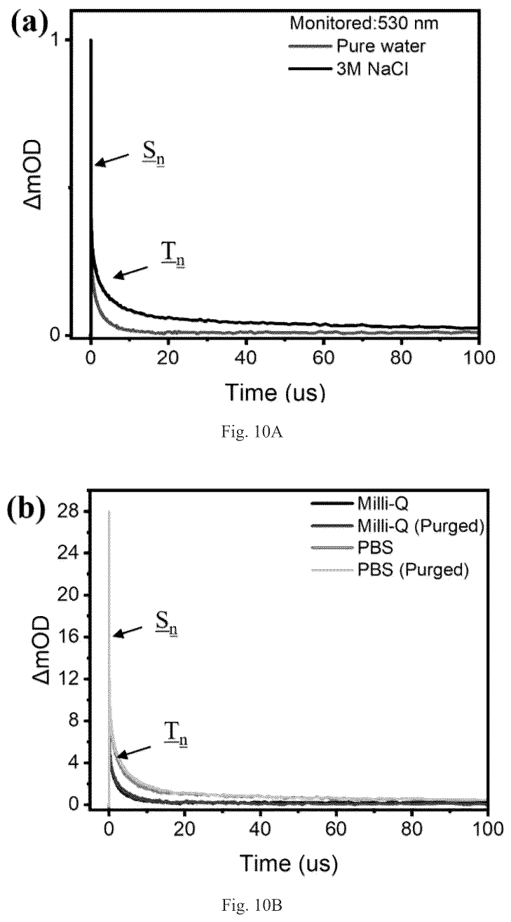

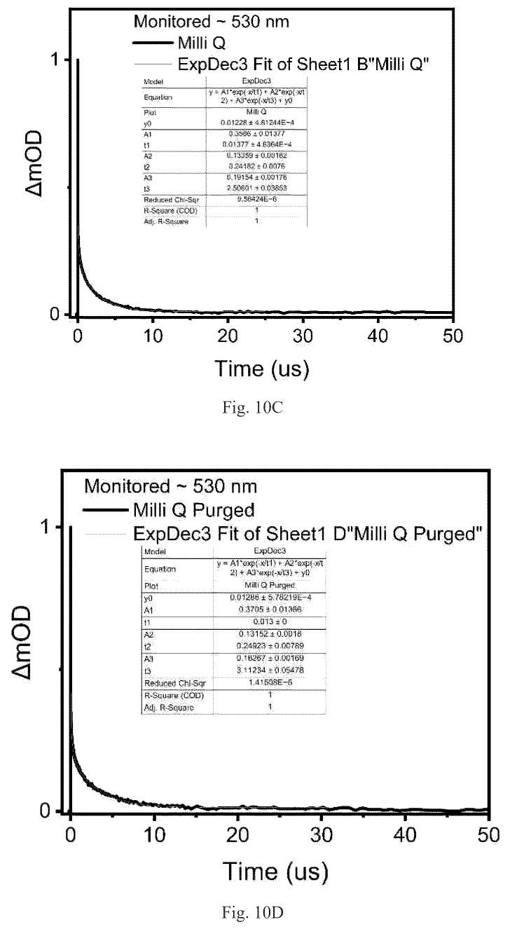

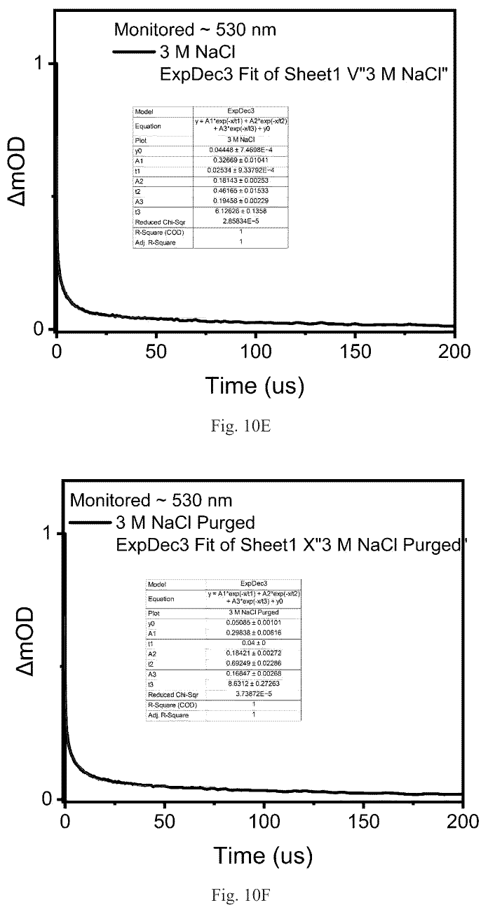

For better understanding, the excited state decay plots for ICTO-BPC were obtained in all the solvents. The data exhibited the existence of multi-component long-lived quantum states in the ρs timescale (FIGS. 10A-10F). The presence of the high quantum density and long-lived quantum states also pointed towards the feasibility of the reverse intersystem crossing (RISC) phenomenon from triplet to singlet excited state (T1-S1), potentially leading to delayed fluorescence property in the compound.

Another interesting piece of information obtained from the transient data was the detecting ketyl radicals, mostly observed as a positive excited state absorption band that spans from 530-700 nm, possible due to benzophenone's triplet-mediated radical generation, which is a doublet (Dₙ) quantum state (FIGS. 9A-9F). In FIGS. 10A-10F, a multi-component decay profile with at least 3 component decay was obtained for ICTO-BPC, demonstrating its multilevel quantum system character by accessing its Sₙ, Tₙ, and Dₙ simultaneously.

Furthermore, to understand the influence of oxygen on these quantum states, the transient absorption measurements in the absence of dissolved oxygen by purging the solutions with argon gas were performed (FIGS. 9A-9F and FIGS. 10A-10F). It was observed that the lifetime of the long-lived components, namely Tₙ, and Dₙ, increased further in the absence of oxygen (FIG. 10B), increasing their quantum coherence at room temperature.

After a detailed investigation and understanding of the quantum state characteristics of the ICTO-BPC in different solvents and in the absence of oxygen, the properties of the excited states under the influence of viscosity were analyzed. Measurement of the quantum state properties under the influence of increasing viscosity demonstrated results in agreement with the quantum states observed in different solvents previously. ICTO-BPC in solutions of varying viscosities retained the multi-component and high-density Tₙ and Dₙ states from the ns to μs timescale at room temperature (FIGS. 11A-11C). This implied the retention of multi-level quantum characteristics in the molecule at room temperature, even in a viscous medium. Analysis done in the absence of oxygen demonstrated a further increase in the lifetime of the multi-component long-lived component, increasing their quantum coherence at room temperature. Therefore, the multi-level quantum character observed for ICTO-BPC in different solutions discussed in the previous section was also obtained when the viscosity of the medium was altered gradually.

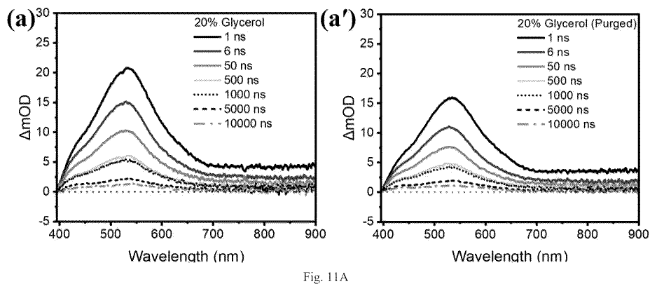

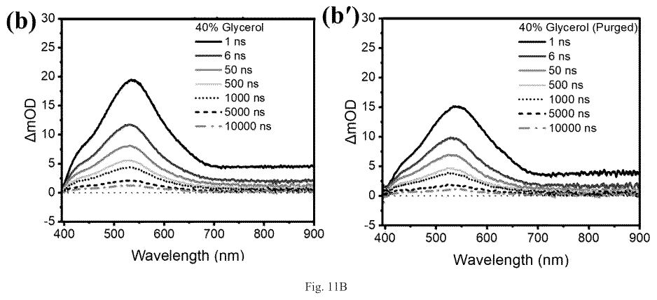

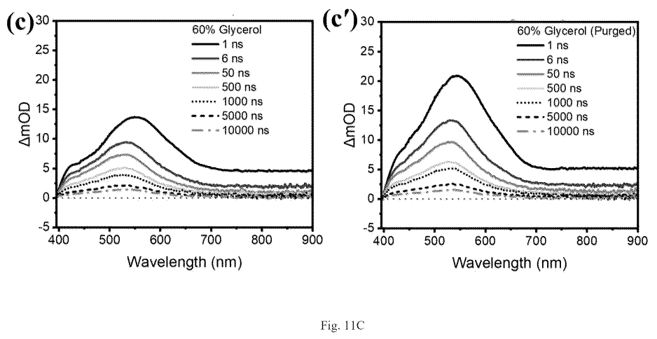

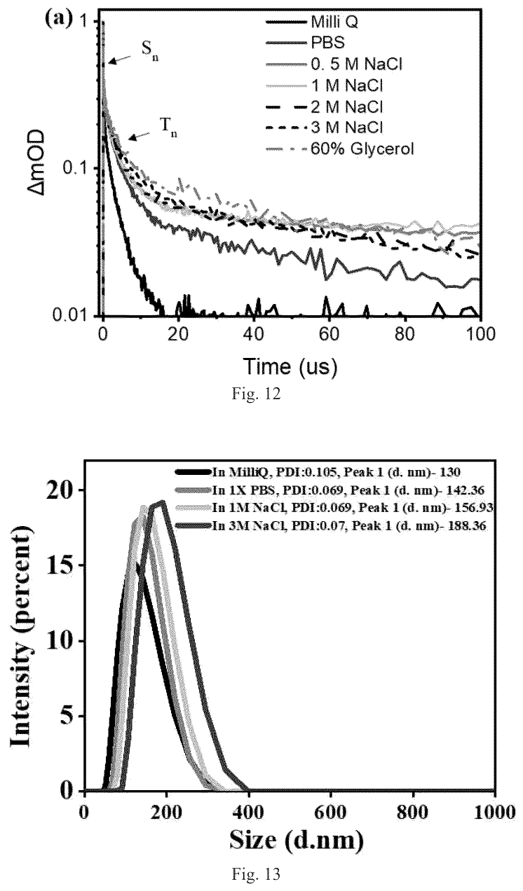

Comparative data plots have been provided in FIG. 12 that show the quantum state properties of ICTO-BPC in solutions of different salt compositions and concentrations, and indicate the increased lifetime of multi-component long-lived quantum states at room temperature upon increasing salt concentration, showing tunable multi-level quantum characteristics in all the different conditions.

ICTO-BPC possesses twisted intramolecular charge transfer (TICT) properties, as demonstrated by the glycerol-water experiment. It also has thermally activated delayed fluorescence (TADF) characteristics, as validated by gated emission measurements and transient absorption measurements. In addition, the ICTO-BPC consists of high T₁ and D₁ density in all the tested solutions with different salt compositions, concentrations, and dielectric constants, suggesting the coexistence of multiple tunable coherent quantum states within the sample at room temperature.

### Example 4

### Investigation of the Photophysical Properties of ICTO-BPC in Liposomes

As ICTO-BPC can spontaneously intercalate into the lipid bilayers in biological systems, the lipid intercalation properties of ICTO-BPC in liposomes were investigated. Liposomes composed of 3:1 ratio of POPC:POPG were used. To that end, initially multilamellar vesicles (MLVs) were prepared with 3:1 ratio of POPC:POPG and 1% ICTO-BPC. The MLVs, when completely dried and free of any organic solvents, were rehydrated using different solvents with different salt compositions and concentrations to understand the implications of these solvents in the intercalation property of the ICTO-BPC and thereby their influence on the photophysical properties. These MLVs were then extruded with 100 nm membranes to obtain liposomes of uniform size distribution. The obtained ICTO-BPC intercalated liposomes were then utilized for investigating various photophysical properties.

To validate the liposome formation, size uniformity, and distribution, the developed liposomes were characterized with the dynamic light scattering (DLS) technique. The DLS data provides information about the hydrodynamic size of any particle in solution. DLS measurements of the liposomes in different solutions indicated monodispersed characteristics as observed by a single peak and polydispersity index (PDI) ranging between 0.06-0.1 (FIG. 13). The hydrodynamic sizes of the different liposomes in the solutions were recorded between 130-188 nm. This study indicated the homogeneous size distribution of the developed liposomes in various solvents.

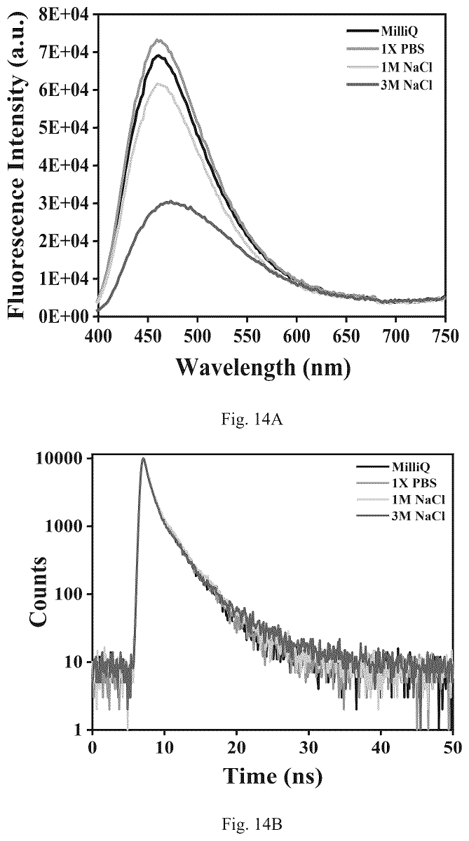

After obtaining liposomes of suitable sizes, the fluorescence emission and fluorescence lifetime properties were examined. The steady-state emission response of the different liposomes demonstrated intense emission peak with peak maxima at 461 nm for MilliQ, 1×PBS and 1 M NaCl, however, the peak was slightly red shifted to 473 nm in 3M NaCl. In terms of emission intensities, ICTO-BPC intercalated liposome in 3M NaCl was less intense when compared with the liposomes in MilliQ, 1×PBS, and 1 M NaCl (FIG. 14A). These differences in the emission wavelength and the emission intensities of the ICTO-BPC intercalated liposomes developed in different solutions correspond to changes in the excited state characteristics of the ICTO-BPC in these solutions.

The fluorescence lifetime studies conducted with the ICTO-BPC intercalated liposomes using the TCSPC technique provided further insights into the excited state properties of the ICTO-BPC in the liposomes (FIG. 14B). The lifetime data displayed a bi-exponential decay with lifetimes ranging between 0.77-0.79 ns. The lifetime of ICTO-BPC in the liposomes was shorter than that of the lifetimes recorded in free solutions.

Detailed insights into the excited state electronic (quantum states) properties of the ICTO-BPC-intercalated liposomes using transient absorption spectroscopy revealed interesting data. The recorded data consistently revealed an important feature: 1) a single-component long-lived transient species was obtained, contrasting the multi-component long-lived transient species present in free solutions (Compare FIGS. 9A-9F, 10A-10F, 11A-11C, 12, and 15A-15C). This reduction from multi-component long-lived quantum states in free solution to a single-component long-lived quantum state in liposomes was attributed to minimizing the Dₙ state density when intercalating into liposomes. The time point measurements indicated that the triplet state is short-lived in comparison to free solutions, as the spectrum recorded at 5 μs showed a minimal band in the spectrum (FIGS. 15A-15C). The spectral data also unveiled critical information about minimizing ketal radicals (Dₙ). The blue-shifter transient band, now closer to 500 nm (in contrast to the band closer to 600 nm recorded in free solutions), has been assigned to triplet states.

### Example 5

Understanding Interactions of ICTO-BPC with Biological Systems

To understand the interactions of ICTO-BPC with other biological systems, the absorption properties of ICTO-BPC using UV-vis spectroscopy in the presence of deoxyribose nucleic acid (obtained from a commercial source) was investigated. The absorption profiles of 20 μM ICTO-BPC in three different solvents (MilliQ, 1×PBS, and 10 mM TRIS) were monitored in the presence and absence of DNA (0.25 and 5 mg/mL). The absorption data revealed an intense absorption band from ~315-460 nm, which clearly red-shifted by 8-10 nm in the presence of DNA w/wo associated increment in the absorbance values (FIGS. 16A-16C). The red-shifted absorption band and the increased absorbance of ICTO-BPC in the presence of DNA demonstrated obvious changes in the ground state electronic properties of the ICTO-BPC in the presence of DNA. Such changes in the ground state electronic character of ICTO-BPC correlate to suitable interactions with DNA.

The steady-state emission study of ICTO-BPC in the presence of DNA in different solvents (MilliQ, 1×PBS, and 10 mM TRIS) was also conducted. The emission profiles of ICTO-BPC in the presence of DNA displayed an intense emission peak with maxima ranging between 522-528 nm (FIGS. 17A-17C). Regarding emission intensity, ICTO-BPC in the presence of DNA in MilliQ and 10 mM TRIS exhibited emission enhancements. In the case of MilliQ, the increase in the emission intensity reached around 2-fold compared to the intensity of ICTO-BPC in the absence of DNA.

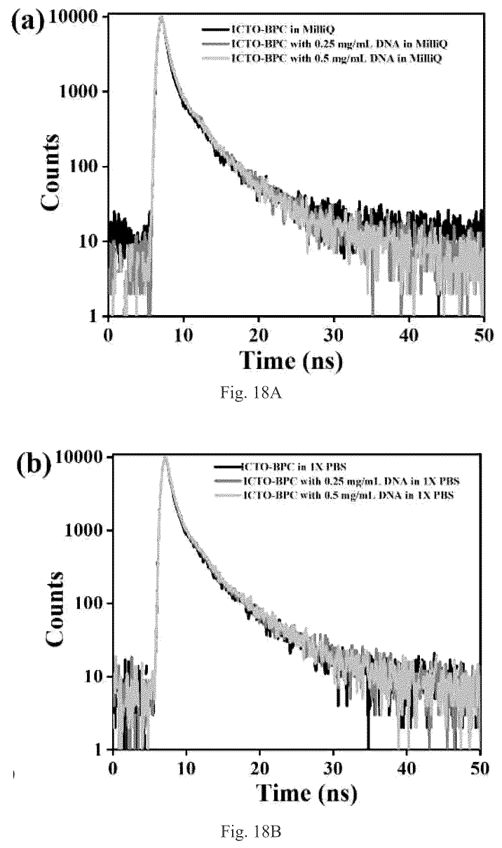

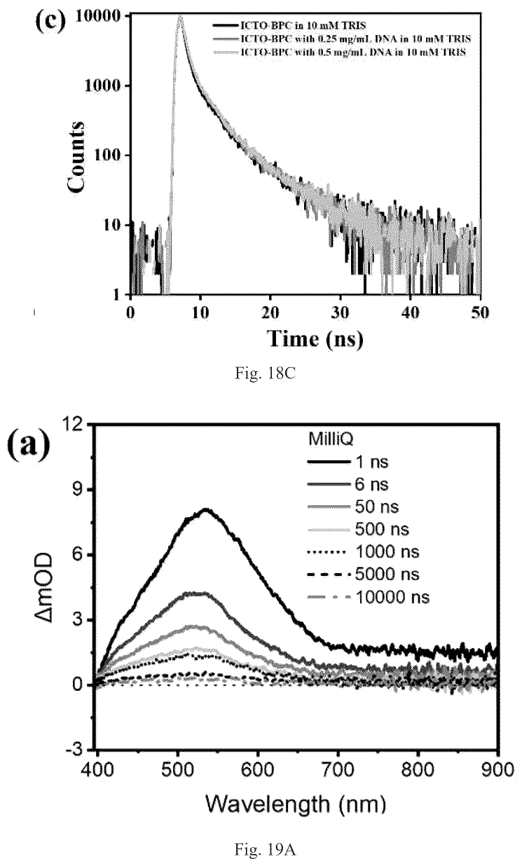

Further investigation of the excited state properties of ICTO-BPC in the presence of DNA was conducted using the TCSPC technique. Using TCSPC, the fluorescence lifetimes of ICTO-BPC were obtained in the presence of DNA in all three solvents used for the previous studies (FIGS. 18A-18C). The lifetime decay data demonstrated bi-exponential decay. Although the calculated lifetimes for the ICTO-BPC in free solution and the presence of DNA in all the tested solvents remained lower than 1 ns, the lifetime values of ICTO-BPC noted in the presence of DNA were slightly higher than that of the free solution. The shorter lifetime values can be attributed to the rapid conversion of the singlet excited state population into other quantum states due to overlapping energies.

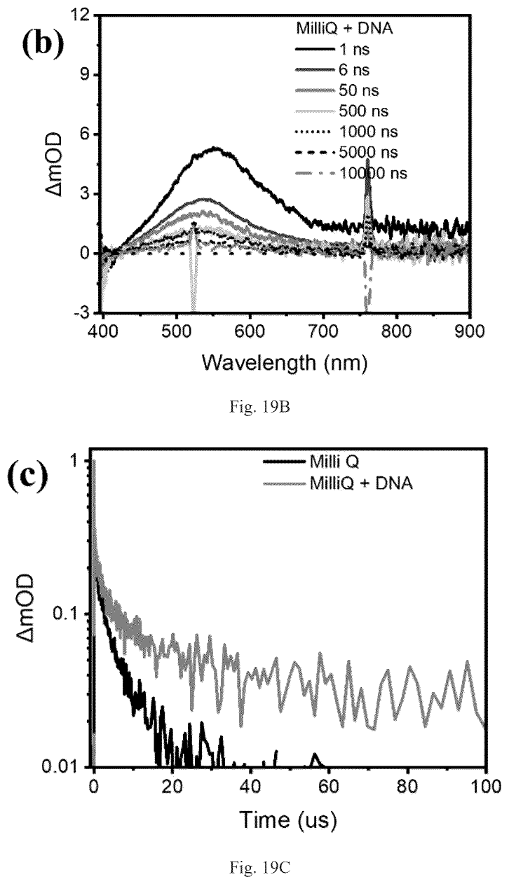

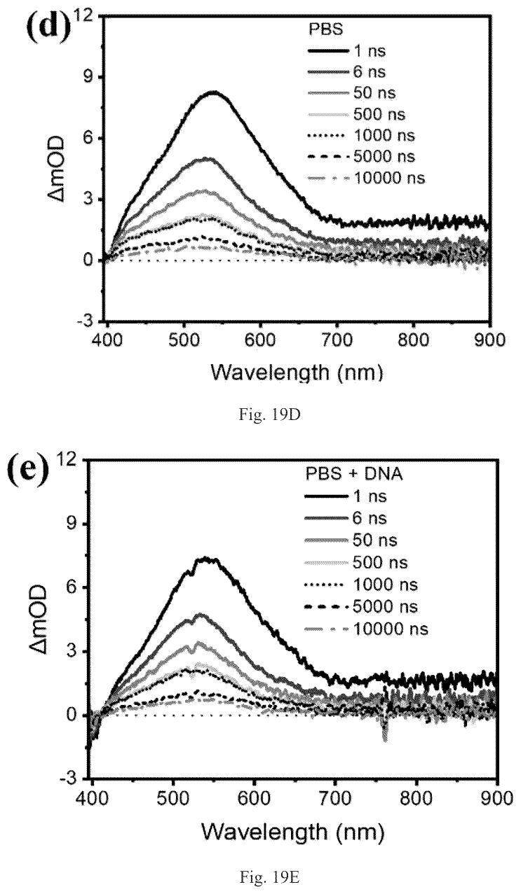

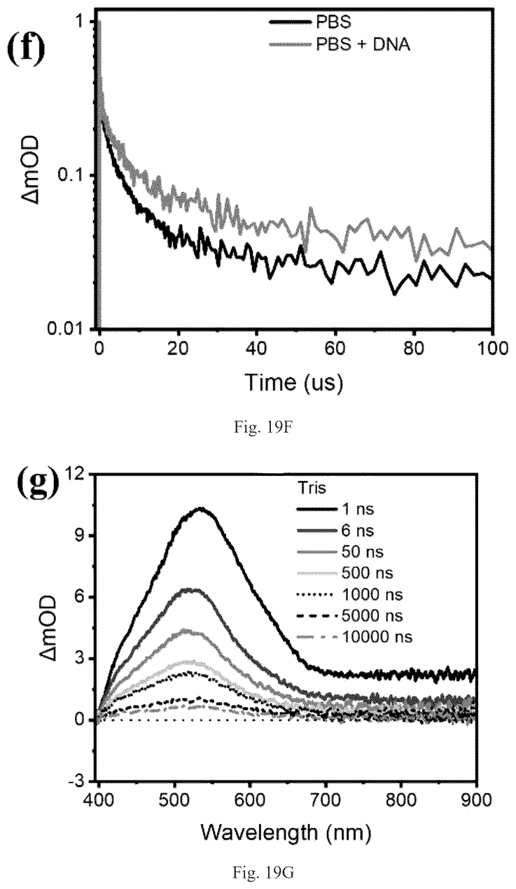

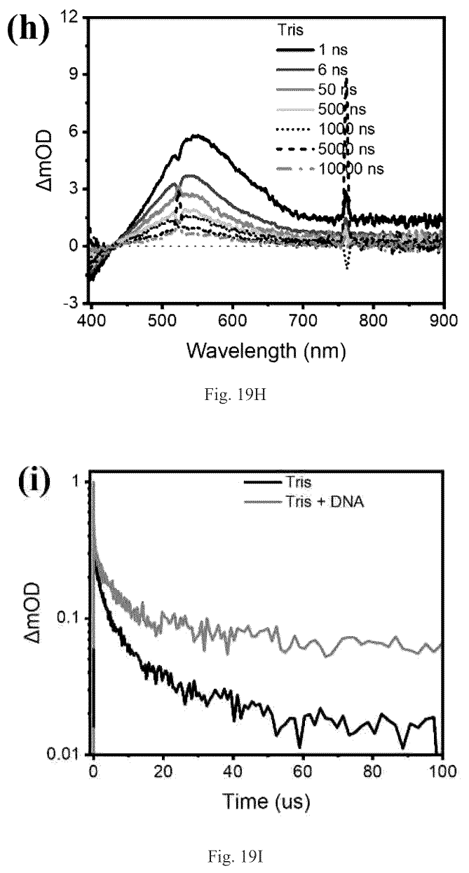

Transient absorption measurements of the ICTO-BPC-DNA systems in different solvents were performed. The positive excited state absorption (ESA) (sometimes called transient species or quantum states) signals recorded in the measurements for ICTO-BPC and the ICTO-BPC with DNA systems indicated the singlet to triplet electronic transitions. Contrary to what was observed in liposomes, an ESA profile consistent with multilevel-quantum characteristics was obtained when interfacing with DNA. For example, monitoring the time point measurements from 1 ns to 10 μs revealed longer lifetimes for the transient species (FIGS. 19A-19I). Moreover, the lifetimes of the multi-component long-lived species were lengthened in the presence of DNA regardless of the solvent tested, indicative of Tₙ and Dₙ coexisting, revealing multi-level quantum phenomena in biological environments (FIGS. 19C, 19F, and 19I).

Another interesting observation noted from the transient data was the wavelength of the obtained peak. As discussed above, the peak position farther than 500 nm or closer to 600 nm is representative of the presence of ketyl radical formed, which has Dₙ character, localized in the benzophenone moiety. Here, the peak position closer to 600 nm suggested the co-existence of the ketyl radical in the ICTO-BPC with the triplet state (FIGS. 19A-19I). The accessibility of the single Sₙ, Tₙ, and the Dₙ in the same system makes it a multi-level quantum system applicable to biological environments, capable of differentiating between DNA and liposome intercalation.

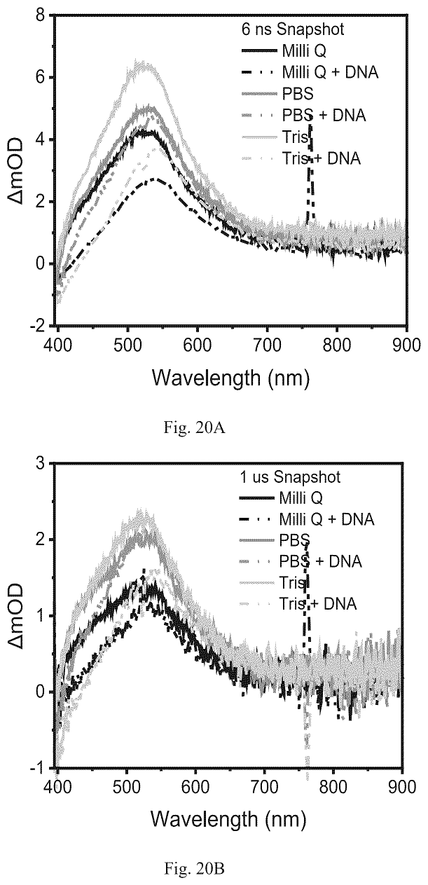

For more precise comprehension, the transient spectra for the changes in the Tₙ and Dₙ quantum states of ICTO-BPC in the absence and presence of DNA, obtained at 6 ns and 1 μs, have been shown in FIGS. 20A and 20B. Note the red-shifted ESA maxima when DNA is present in the solution, indicating a strong interaction between ICTO-BPC and the DNA, which can be assessed optically by monitoring such quantum states.

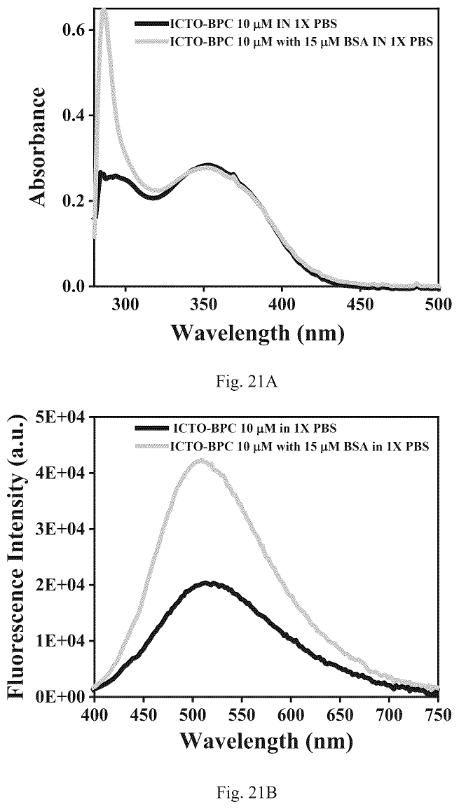

To understand the interactions of ICTO-BPC with other biological systems, the absorption properties of ICTO-BPC using UV-vis spectroscopy in the presence of Bovine Serum albumin (BSA) protein (obtained from a commercial source) were investigated. The absorption profiles of 10 μM ICTO-BPC in 1×PBS were monitored in the presence and absence of 15 uM BSA. The absorption data revealed an intense absorption band from ~315-460 nm (FIG. 21A).

The steady-state emission study of ICTO-BPC in the presence of BSA in 1×PBS was also conducted. The emission profiles of ICTO-BPC in the presence of BSA displayed an intense emission peak with maxima ranging between 522-528 nm (FIG. 21B), two times more intense than in a BSA-free environment.

### Example 6

### Continuous-Wave Electron Paramagnetic Resonance (EPR) Spectroscopy.

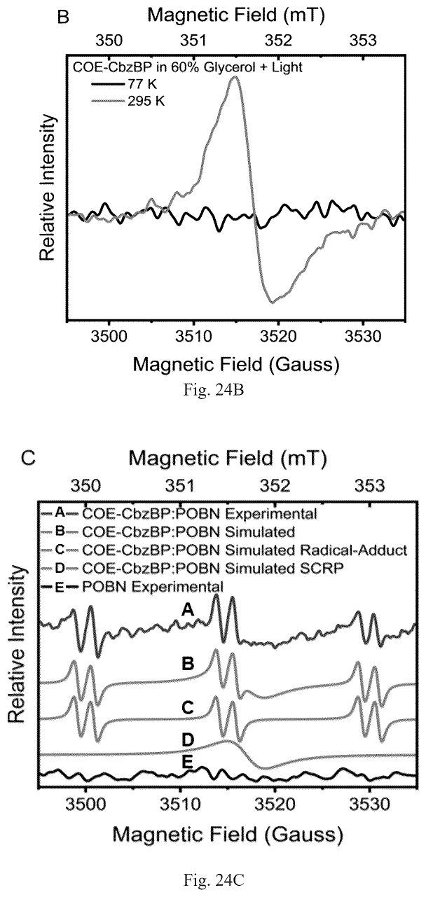

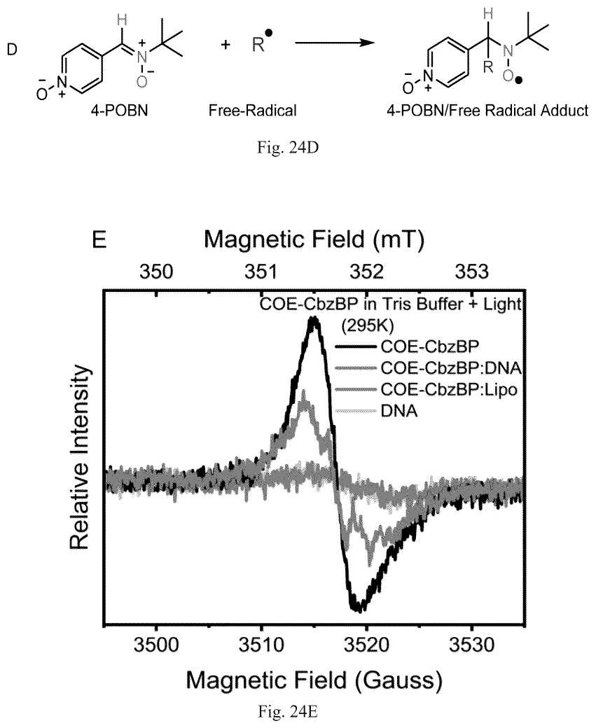

The presence of photogenerated SCRP and its biophysical implementation can be initially assessed using CW-EPR spectroscopy. Measurements were carried out with an X-band (9.8 GHz) under oxygen-free conditions with (light) and without (dark) irradiation. To minimize UV-induced photochemistry in biomolecules, a Corning 7740 optical filter was used to transmit light above 300 nm. As shown in FIG. 24A, a positive singlet EPR signal with g=2.0049 was detected for the COE-CbzBP in aqueous conditions only under irradiation. This spin-polarized signal was completely absent in the dark, consistent with photogeneration of radical pairs and molecular triplets. To gain deeper insight into the origin of such spin-polarized signal, comparative CW-EPR measurements were carried out at cryogenic conditions (77 K), see FIG. 24B. Interestingly, the spin-polarized EPR signal observed at room temperature was absent at 77 K despite clear evidence of molecular triplet formation from the phosphorescence measurements (FIG. 23A). These findings suggest that thermal energy is required to access such a spin-polarized state, a behavior consistent with the presence of an activation barrier for attaining the TICT geometry precursor of efficient singlet-triplet manifold mixing.

To authenticate the formation of photogenerated radical intermediates, radical-specific trapping experiments were performed using 4-POBN (40× to that of COE-CbzBP), a well-known spin-specific sequestrator, see FIG. 24C. Upon irradiation of COE-CbzBP in the presence of 4-POBN, the X-band EPR spectrum acquired at room temperature exhibited a well-resolved triplet-of-doublets hyperfine pattern at g=2.0061, consistent with the formation of a 4-POBN-radical adduct, along with an additional signal at g=2.0049. The triplet signal from the 4-POBN-radical adduct arises from hyperfine coupling with the nitrogen nucleus of POBN with aN=15.12G, while each line is further split into a doublet by the β-hydrogen atom with aH=1.96G. These spectral parameters are consistent with literature-reported values for carbon-centered POBN-adducts formed, confirming the successful capture and characterization of organic radicals under photoirradiated conditions. No signal was obtained for the neat POBN control sample (FIG. 24C) upon irradiation.

Taken together, these observations suggest that the detected EPR signals in the presence of POBN may originate from two possible mechanisms: (1) partial dissociation of the SCRP, releasing free radicals that are then trapped by POBN; or (2) disruption of the SCRP state by POBN itself, thereby exposing the individual radicals to detection, see FIG. 24D for mechanistic insights. The current understanding is that the observed EPR spectra most likely originate from a charge-separated radical pair [Cbz^{·+}-BP^{·-}] with triplet character rather than molecular triplets, stabilized by hyperconjugation, resonance delocalization, and steric hindrance. Similar EPR spectral features have been independently reported for the ketyl and cationic radical of benzophenone and carbazole, respectively. Interestingly, those spin-polarized signals, doublets or triplets, are often challenging to detect at room temperature and usually require pulsed EPR techniques or low-temperature stabilization. It is important to note that the triplet excited state of benzophenone is not likely responsible for the observed signal here, as it is characterized by strong zero-field splitting and anisotropy that requires specialized techniques, such as optical detection of magnetic resonance (ODMR), for spectroscopic observation. Conversely, these results more closely resemble reported persistent, isotropic EPR signals with g-values between 2.003 and 2.005 upon UV irradiation of crystalline benzophenone-urea assemblies. These signals were attributed to ketyl radical pairs stabilized through resonance within a rigid solid-state matrix. In the case of COE-CbzBP, the formation of a thermally accessible spin-polarized SCRP state is attributed to the small ΔE_{ST} value of 0.12 eV, which corresponds to a J_{RP} of 0.06 eV. These parameters enable thermal population of the triplet manifold and facilitate spin-mixing, consistent with design principles for optically addressable qubits.

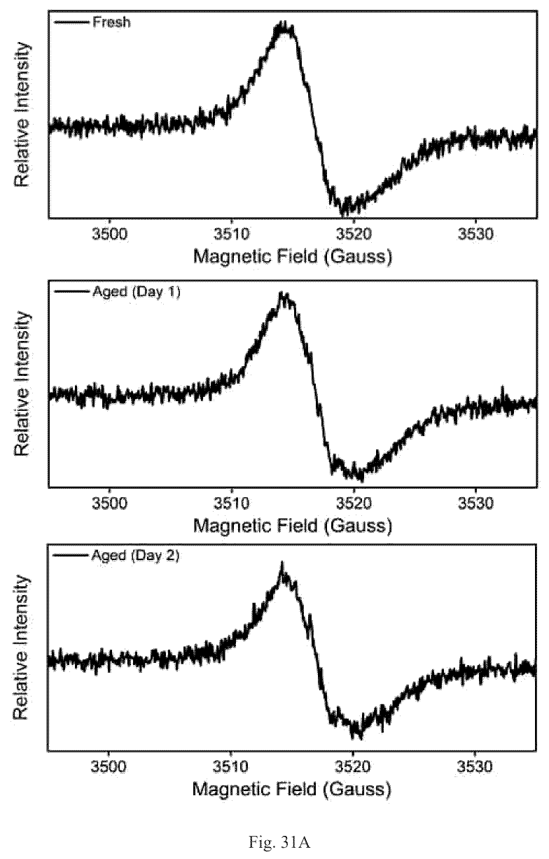

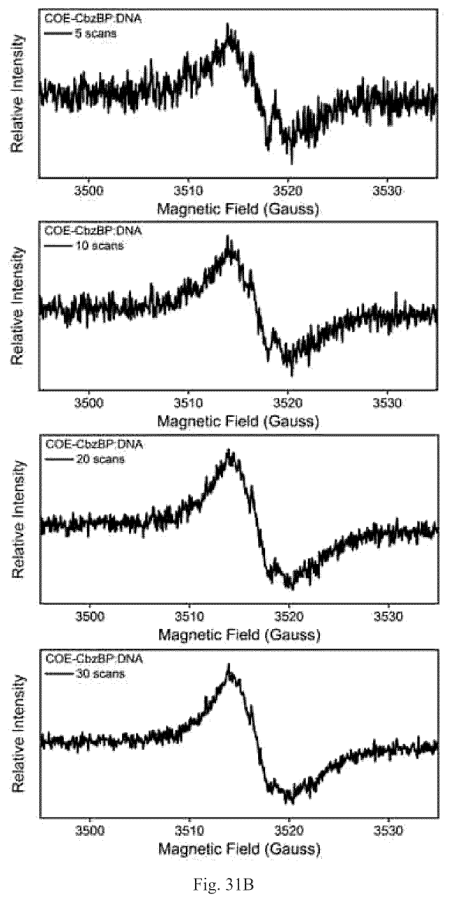

Next, it was evaluated how different biological microenvironments influence the room-temperature spin-polarized EPR signal of the COE-CbzBP, see FIG. 24E. The most consistent results were obtained when the COE-CbzBP:DNA solution was prepared and allowed to interact (aged) overnight (FIGS. 31A and 31B). The doublet signal was more intense during the first scans in EPR, so FIG. 31B shows the scan-dependent EPR characterization of COE-CbzBP:DNA. The signal splitting gradually decreases in intensity with each scan (FIG. 31B). Each scan requires approximately 80 seconds to complete. This behavior is attributed to cumulative microwave-induced heating and vibrational effects that weaken the COE-CbzBP:DNA interactions, leading to partial dissociation of the complex. The released (unbound) COE-CbzBP species then contributes to the overall EPR signal.

It was interesting to note that the photogenerated spin-polarized signal at g=2.0049 of the COE-CbzBP splits upon association with DNA, yielding an apparent doublet (see FIG. 24E). It has been suggested that the electric field of DNA can modulate the change in transition dipole moment of charge-transfer chromophores, leading to binding-mode-dependent changes in their two-photon absorption (TPA) cross-sections, despite similar shifts in transition frequency and permanent dipole moment (Δμ=μₑ−μ_{g}). From a quantum mechanics perspective, the TPA cross-section depends not only on changes in Δμ, but is square-dependent on the transition dipole matrix elements between these states. Recent reports estimate that the static electric field strength within 2 nm of DNA can reach up to 109 volts per meter (V·m⁻¹). Consequently, the evident EPR doublet signal observed for COE-CbzBP upon DNA association is attributed not only to the strong local electric fields of DNA affecting Δμ in the ground state (Stark effect, see FIG. 16) and the transition dipole moment of COE-CbzBP, but also the spin polarization of the photogenerated SCRP by electric field-spin coupling, influencing J_{R} and Ax when in its TICT form, see FIG. 22E.

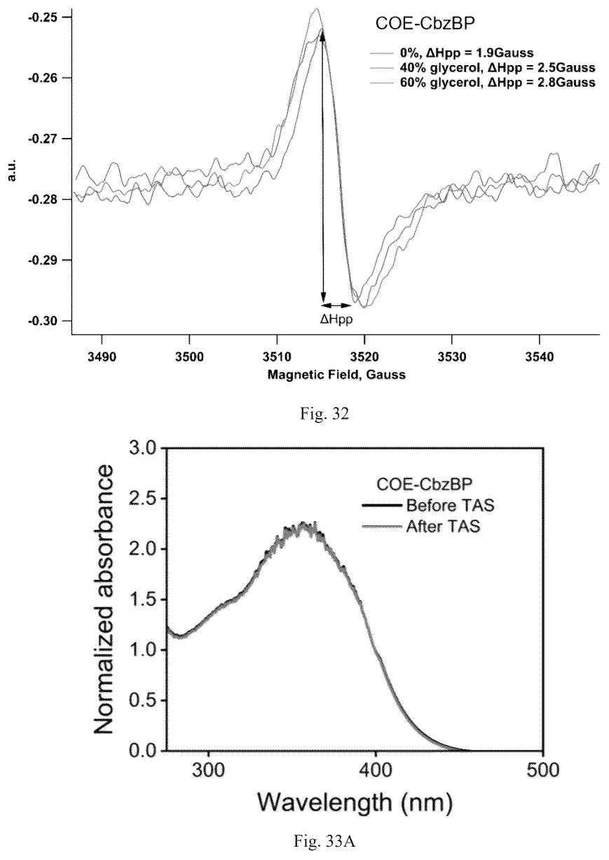

This effect became more apparent when COE-CbzBP was intercalated into lipid bilayers, which, like DNA, can generate static electric fields due to the negatively charged phosphate head groups. Such a spin-polarized EPR signal is not obtained in these biological environments despite favorable ΔG_{CS}, presumably due to the planar zwitterionic configuration that COE-CbzBP adapts in lipid bilayers, see FIG. 24E and FIG. 22G. It is worth noting that no signal was detected when DNA alone or lipids (without COE-CbzBP) were characterized via EPR under any condition. To rule out viscosity effects, experiments were conducted in aqueous glycerol mixtures. Increased glycerol concentration led to spectral line broadening, not spectral splitting. For example, linewidth (ΔHₚₚ) increased from 1.9 G in water to 2.8 G in 60% glycerol (FIG. 32), which might indicate that the spin-lattice and spin-spin relaxation dynamics of the charge-separated radical pair are shortened in glycerol. These results highlight the predominant role of electrostatic, rather than rheological, effects exerted by DNA on the spin dynamics of COE-CbzBP.

### Example 7

### Transient Absorption Spectroscopy.

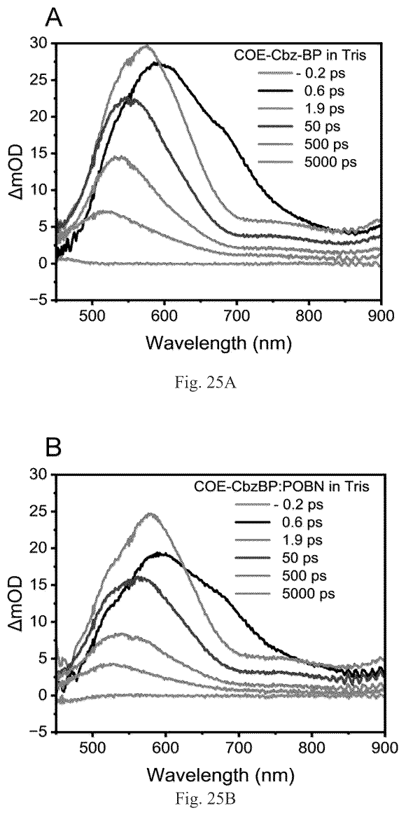

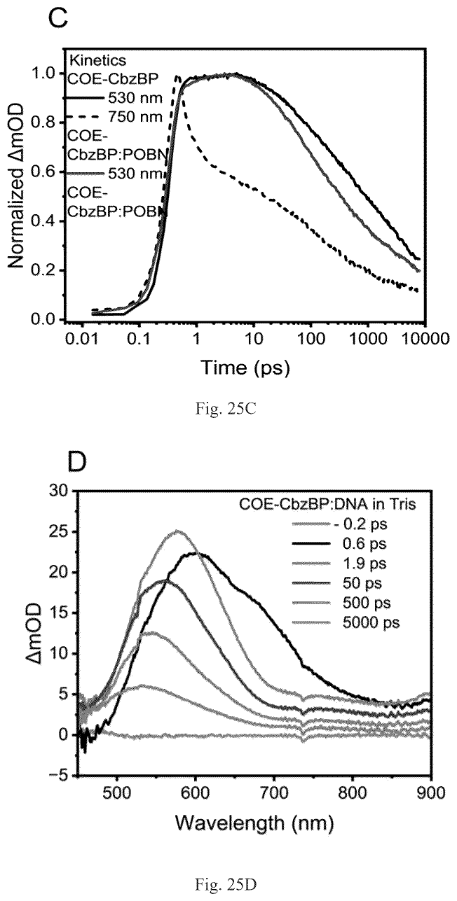

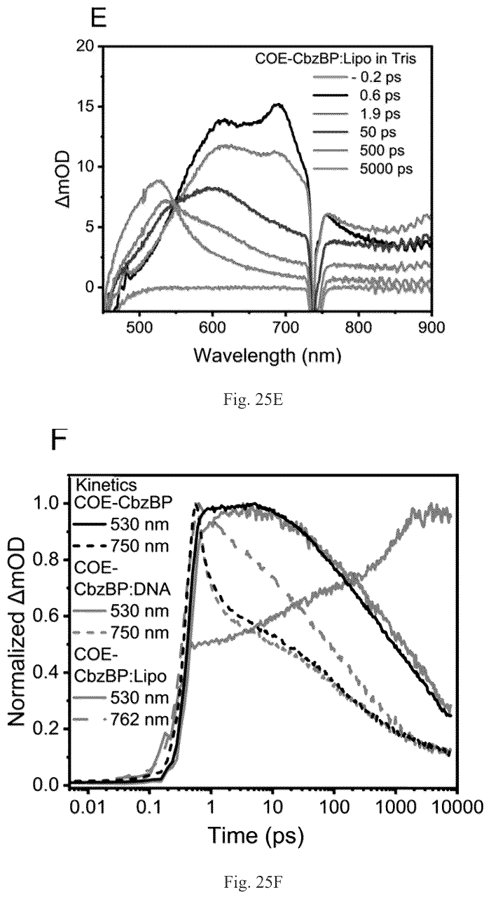

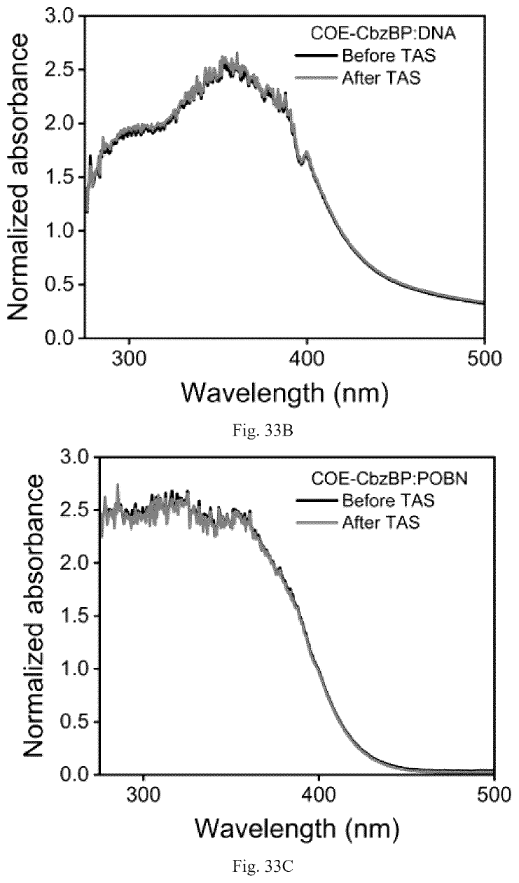

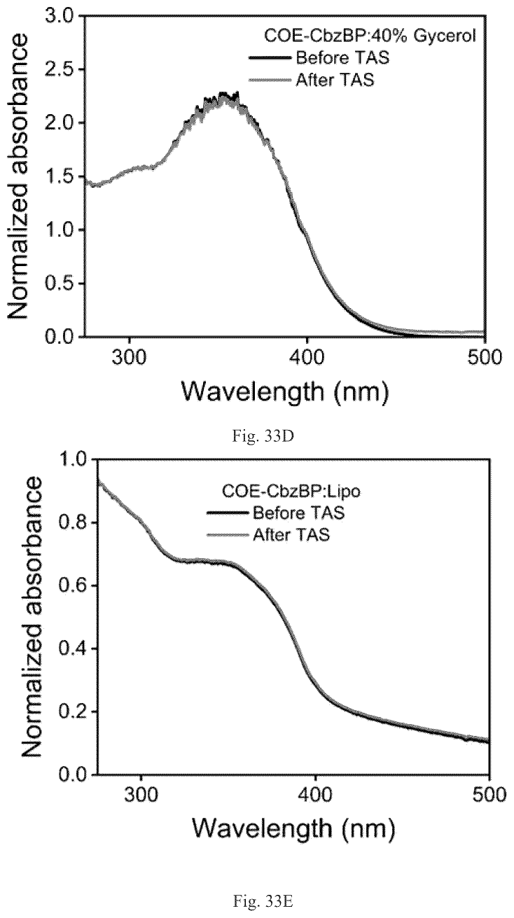

Transient absorption spectroscopy (TAS) experiments, conducted under conditions analogous to those used in CW-EPR, were performed to confirm and investigate the formation and environmental sensitivity of the charge-separated radical pair in COE-CbzBP. It is anticipated that upon excitation, spectral features associated with the [BP^{·-}]* and [Cbz^{·+}]* radical pairs will be present in the excited state, as it has been reported in other organic SCRP. To evaluate the presence of such radical pairs, COE-CbzBP was first characterized from femtosecond (fs) to nanosecond (ns) timescales under oxygen-free conditions at room temperature using 370 nm excitation in aqueous and biologically relevant environments, see FIGS. 25A-25F. Absorption measurements were taken before and after each TAS measurement to confirm that the sample remained stable (i.e., no degradation) during characterization, see FIGS. 33A-33E.

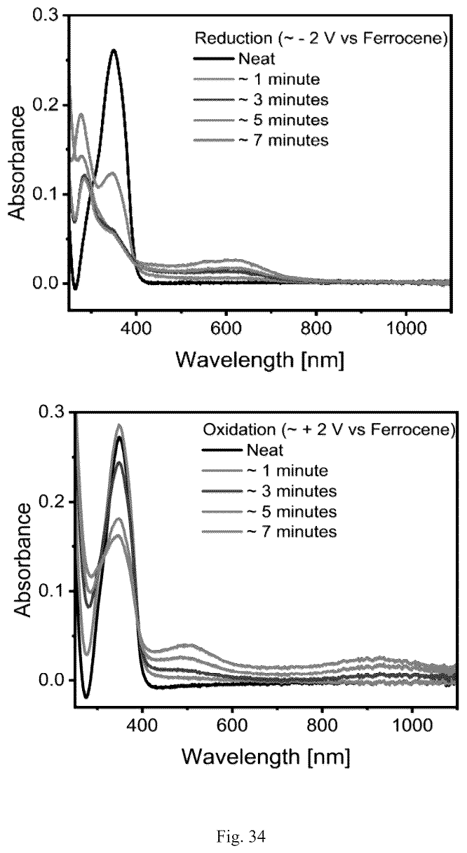

In all cases, a strong excited-state absorption (ESA) band centered between 600-700 nm was observed within 1 μs, corresponding to the localized-excitation formation of ¹[Cbz-BP]*. This ESA rapidly evolved depending on the medium. For example, in Tris-HCl buffer (FIG. 25A), two new ESA bands evolved, one more intense ESA within 450-700 nm and another, less intense, spanning 750-900 nm. A subtle but appreciable risetime (<2 μs) was observed for the high-energy band, which migrates towards 530 nm and decays beyond 5000 μs. Such intense ESA could be attributed to the absorption of [BP^{·-}]* and [Cbz^{·+}]* charge-separated radical pairs, while the low-energy ESA band is consistent with the absorption of [Cbz^{·+}]*. These features match previously reported spectra and timescales of similar TICT scaffolds, including isolated or conjugated benzophenone and carbazole radical ions. In addition, these assignments were corroborated by spectroelectrochemistry, see FIG. 34. A 400-700 nm absorption profile appeared upon reduction of COE-CbzBP, while positive potentials yielded two stronger bands at 400-600 nm and 750-1000 nm. Therefore, the most representative ESA of the radical pair, attributed to ¹[Cbz^{·+}-BP^{·-}]*, is within 400-600 nm. The radical pair nature of COE-CbzBP was further supported by spin-trapping experiments with 4-POBN (40×), which produced similar but quenched and faster ESA decay kinetics (FIGS. 25B-25C), confirming ¹[Cbz^{·+}-BP^{·-}]* formation on the picosecond timescale with a longer-lived component extending beyond the nanosecond regime.

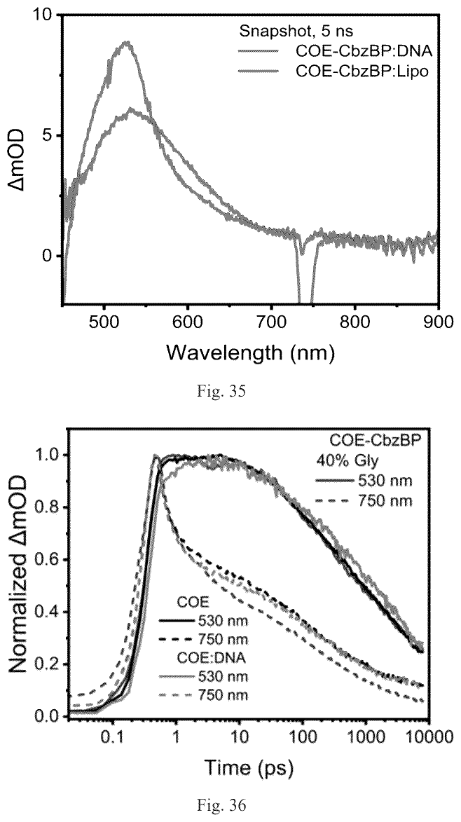

Next, the ¹[Cbz^{·+}-BP^{·-}]* in DNA and lipid bilayer environments was evaluated to contextualize such dynamics further with the results obtained by CW-EPR, see FIGS. 25D-25F. Concerning DNA environments, a lengthening in the ESA profile relative to that in DNA-free conditions was obtained, see FIG. 25F. Interestingly, in liposomes, a blue-shifted ESA band with a more pronounced long-lived risetime was detected (see FIGS. 25E-25F). Prior studies on benzophenone have shown that such blue-shifted ESAs (towards 500 nm) are associated with molecular triplet transitions, whereas red-shifted bands (towards 540 nm) correspond to radical transitions, see FIG. 35. Thus, the red-shifted ESA upon DNA-association is consistent with the presence of radicals, whereas lipid environments favor planar configurations and yield molecular triplet states. Faster ¹[Cbz^{·+}-BP^{·-}]* kinetics than those in aqueous conditions were observed when COE-CbzBP was analyzed in glycerol solutions (FIG. 36). This ESA kinetic behavior correlates with the broader ΔHₚₚ observed in the EPR spectra, indicative of faster spin-lattice and spin-spin relaxation dynamics.

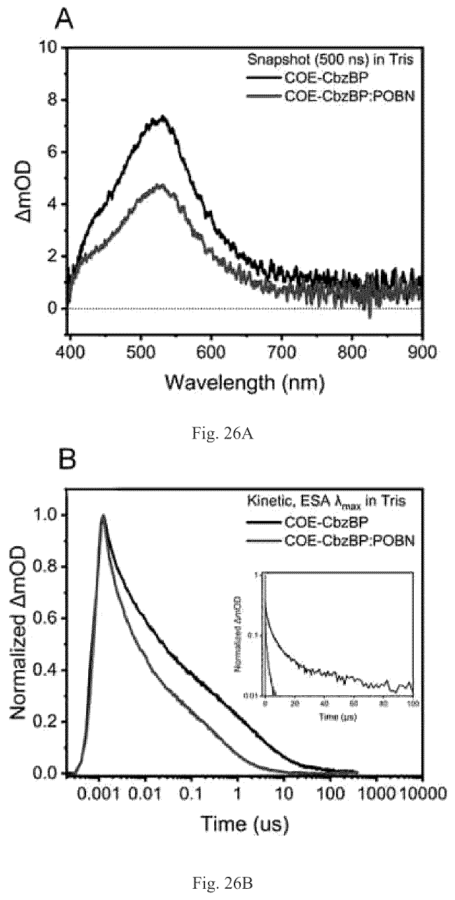

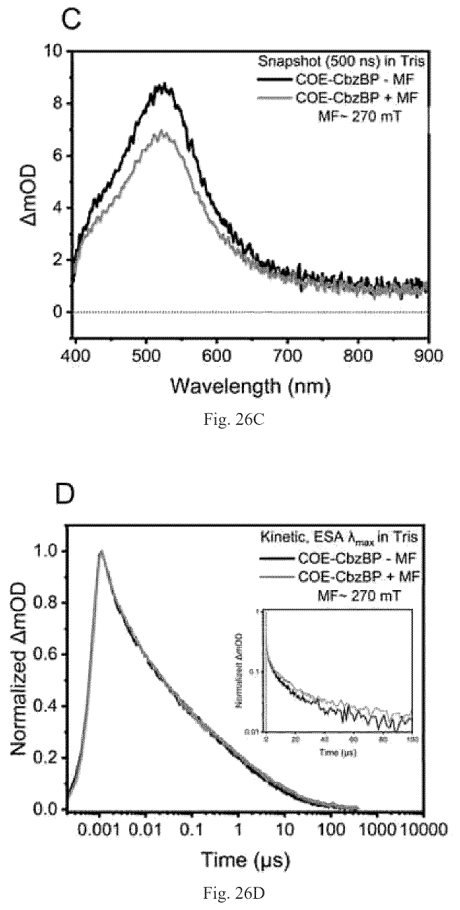

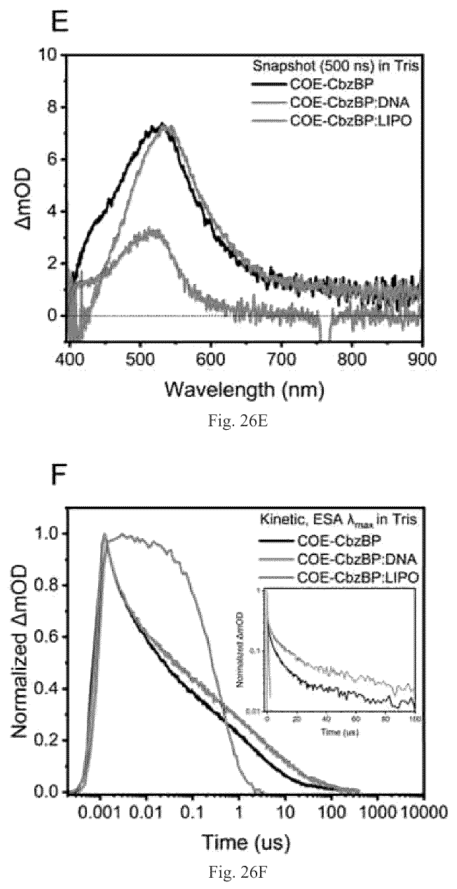

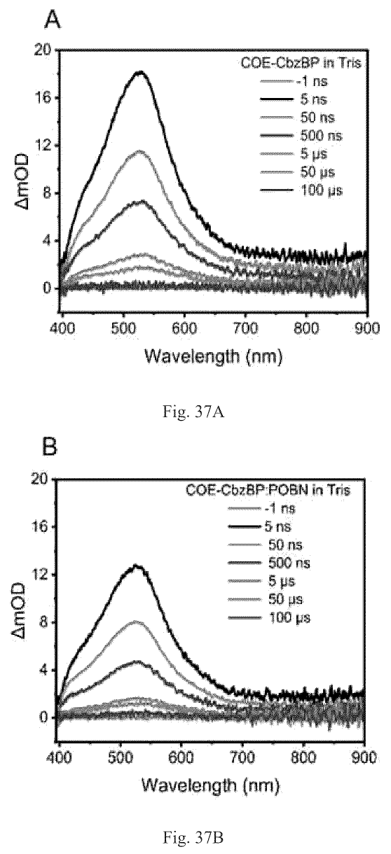

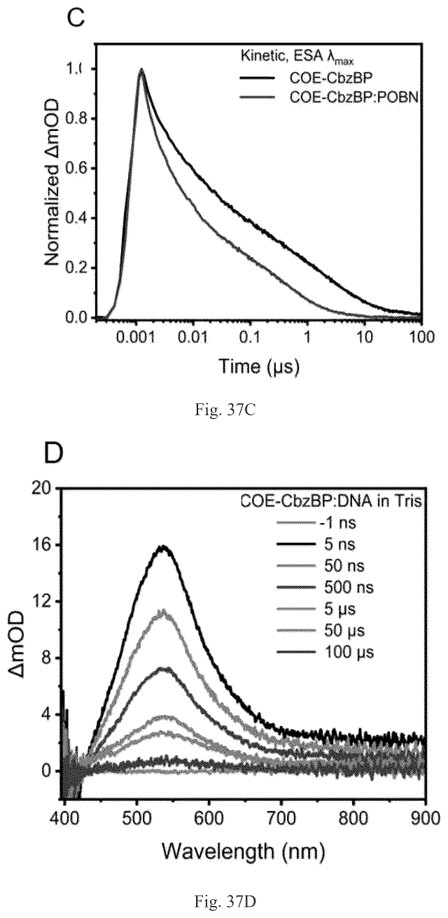

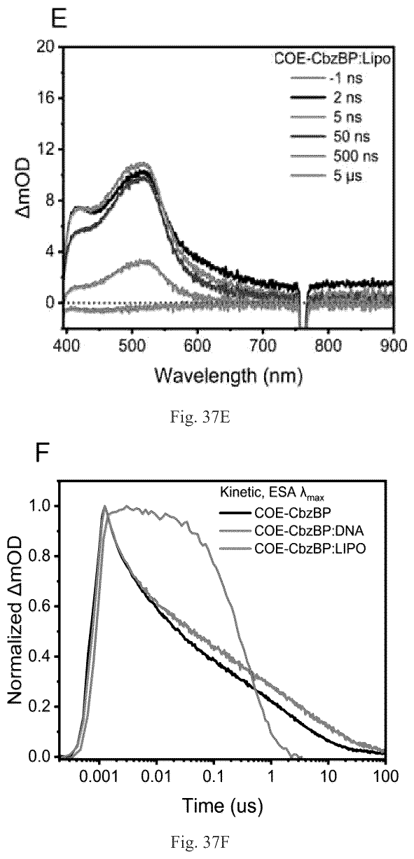

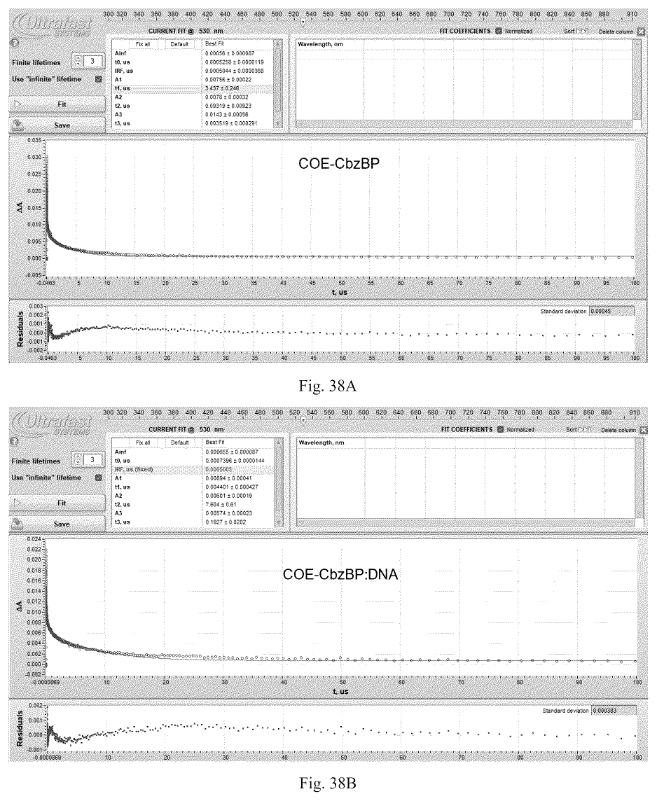

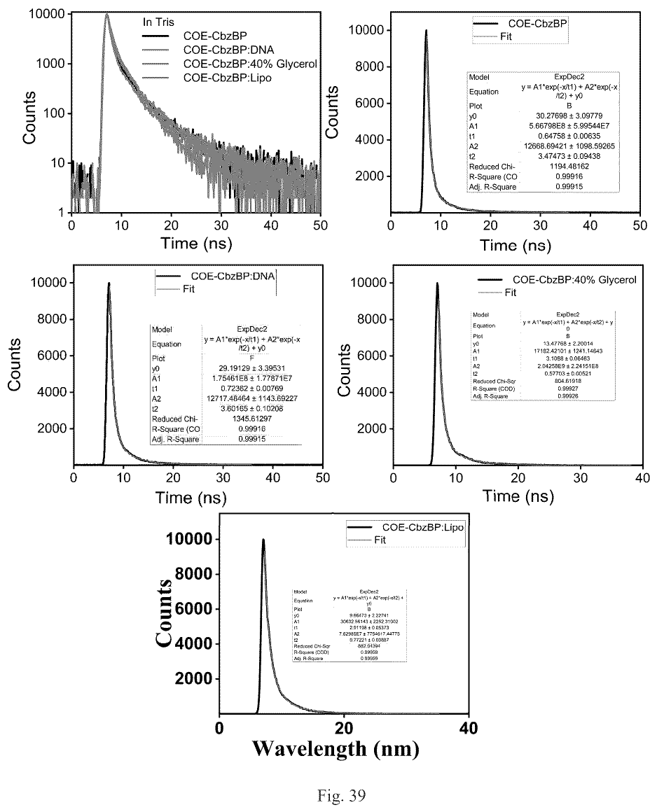

To investigate and resolve the ¹[Cbz^{·+}-BP^{·-}]* decay dynamics, TAS was extended to the nanosecond (ns) and microsecond (s) timescales (FIGS. 26A-26F and Table 7). Snapshots at 500 ns under different conditions are shown in FIG. 4, while their ESA profiles at different timescales are shown in FIGS. 37A-37F. Note that the most noticeable ESA kinetic differences are within these timescales. Under neat aqueous conditions, a multi-component ESA decay was observed, best fit by three exponential components: a fast initial decay in the first 20 ns (τ₁=3.5 ns±0.2 ns), followed by two longer-lived decays spanning the s timescale (T₂=0.093±0.009 s and τ₃=3.4±0.6 μs), see FIGS. 38A and 38B. This first decay component was attributed to the radical-pair intersystem crossing mechanism to form ³[Cbz^{·+}-BP^{·-}]* from ¹[Cbz^{·+}-BP^{·-}]* (rate, k_{RP-ISC}=2.86×10⁸ s⁻¹), competing with ¹[Cbz^{·+}-BP^{·-}]* charge recombination and fluorescence channels to the ground state. This assignment becomes more apparent when one notes that the τ₁ profile and value closely mirror the bi-exponential fluorescence lifetime of COE-CbzBP (τ_{F1}=0.646±0.006 ns and τ_{F2}=3.47±0.09 ns), see FIG. 39. However, given its low Φ (~0.03), the corresponding radiative rates (5.17×10⁷ s⁻¹ and 9.63×10⁶ s⁻¹) are at least an order of magnitude smaller than k_{RP-SC}, further supporting the assignment and explaining the sizable ³[Cbz^{·+}-BP^{·-}]* population.

The τ₂ and τ₃ components are attributed to charge recombination of ³[Cbz^{·+}-BP^{·-}]* via back-electron transfer (rate, ³k_{CRT}=1.08×10⁷ s¹), producing ³[Cbz-BP]*, which subsequently decays to the ground state via non-radiative decay (rate, ³ k_{NR}=3.4×10⁶ s⁻¹), see FIGS. 26B, 38A, and 38B. The τ₂ recombination timescale via back electron transfer of ³[Cbz^{·+}-BP^{·-}]* is consistent with previously reported spin-polarized radical pair lifetimes observed at room temperature in other probes with qubit-like properties. Upon addition of 4-POBN to the COE-CbzBP solution, its ESA intensity and lifetimes in the microsecond range were quenched and shortened through spin-sequestration (FIGS. 26A-26B), consistent with the CW-EPR results. It is important to highlight that a reduction in the ESA intensity and lengthening in the kinetic profile of COE-CbzBP was observed when a permanent magnet was used during its characterization, see FIGS. 26C-26D. It is important to note that both a reduction in ESA intensity and a lengthening of the COE-CbzBP kinetic profile were observed when a permanent magnet was placed near the sample during characterization (FIGS. 26C-26D). The magnet provides ~550 mT at its surface, and the effective magnetic field at the pump-probe interaction region, accounting for the sample-to-magnet standoff distance, was measured to be ~270 mT using a Gaussmeter. This MFE sensitivity is consistent with photogenerated SCRP dominating the excited-state dynamics of COE-CbzBP.

TABLE 7
Summary of the electronic rates of COE-CbzBP in different
microenvironments, obtained from the ns TAS.

| | ¹k_{CS} | ¹k_{F} or ¹k_{CRS} | k_{RP-ISC} | ³k_{CRT} | k_{ISC} | ³k_{NR} |
|---|---|---|---|---|---|---|
| Environment | (s⁻¹) | (s⁻¹) | (s⁻¹) | (s⁻¹) | (s⁻¹) | (s⁻¹) |
| COE-CbzBP | ~5 × 10¹² | 5.17 × 10⁷ | 2.86 × 10⁸ | 1.08 × 10⁷ | n/a | 3.4 × 10⁶ |
| | | 9.63 × 10⁶ | | | | |
| COE- | ~5 × 10¹² | 4.62 × 10⁷ | 2.27 × 10⁸ | 5.21 × 10⁶ | n/a | 1.32 × 10⁵ |
| CbzBP:DNA | | 9.28 × 10⁶ | | | | |
| COE- | ~5 × 10¹² | 5.26 × 10⁷ | 8.745 × 10⁸ | 1.14 × 10⁷ | n/a | 3.06 × 10⁶ |
| CbzBP:Gly | | 9.68 × 10⁶ | | | | |
| COE- | n/a | 1.12 × 10⁸ | n/a | n/a | ~5.0 × 10⁸ | 5.56 × 10⁶ |
| CbzBP:Lipo | | 2.79 × 10⁷ | | | | |

Charge separation (¹k_{CS}); fluorescence rate (k_{F}); Singlet charge recombination via back electron transfer (¹k_{CRS}); Radical pair intersystem crossing (k_{RP-ISC}); Triplet charge recombination via back electron transfer (³k_{CRT}); Intersystem crossing (k_{ISC}); Non-radiative ISC to the ground state(³k_{NR}).

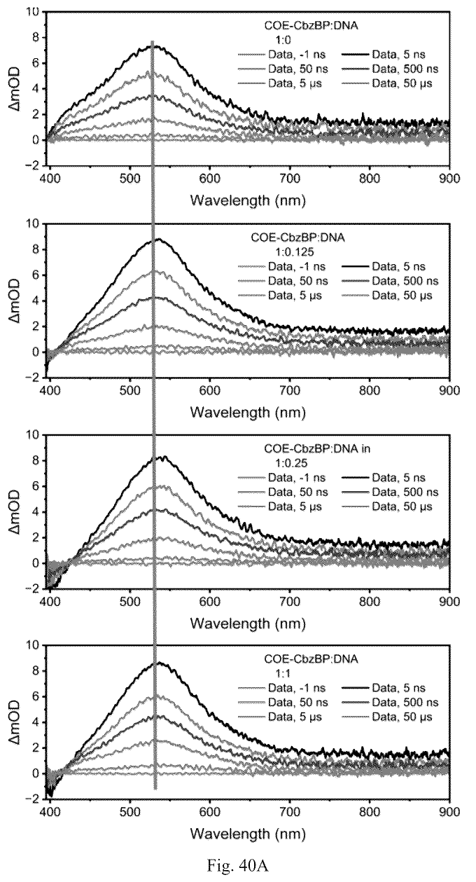

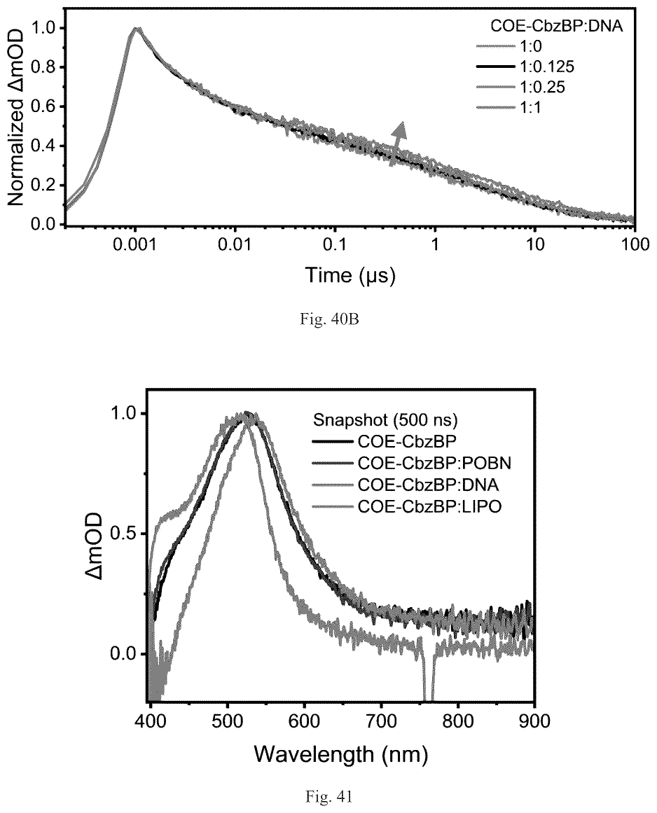

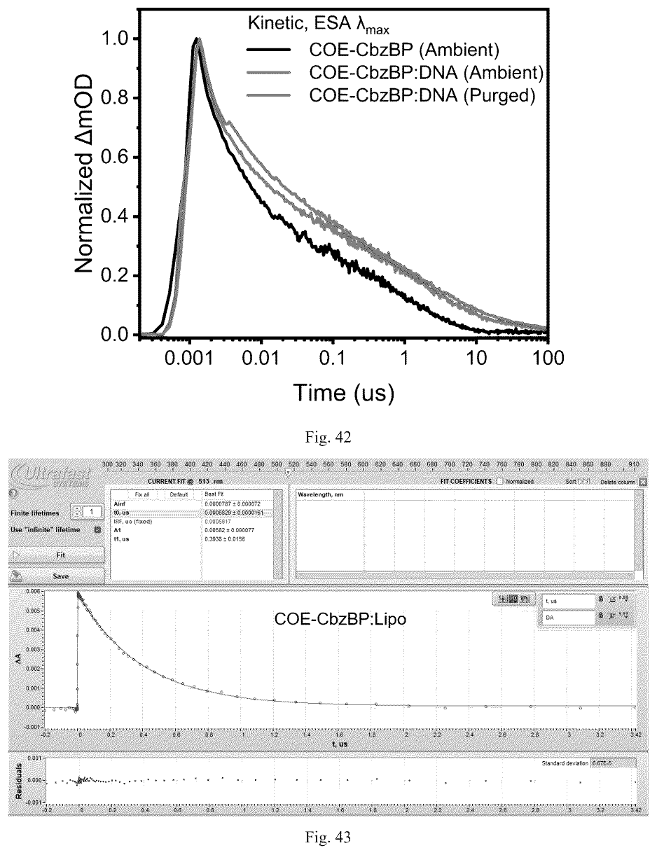

When COE-CbzBP was interfaced with biomolecules, similar ESA bands to those characterized with the fs TAS were observed and kinetically characterized, see FIGS. 26E-26F and FIGS. 37A-37F. Upon titration with DNA (FIGS. 40A and 40B), a systematic red shift (>10 nm) in the ESA assigned to ³[Cbz^{·+}-BP^{·-}]* was observed (see FIG. 26E and FIG. 41), accompanied by a concomitant prolonged multi-component decay (see FIG. 26F and FIGS. 40A and 40B). These results demonstrate that obtaining a redshifted ESA with concomitant long-lived decay is DNA concentration dependent. Under optimized COE-CbzBP:DNA conditions (1:1), shown in FIGS. 26E-26F, the T₁ assigned to RP-ISC remained similar (τ₁=4.4 ns±0.4 ns; rate, k_{RP-ISC}=2.27×10⁸ s⁻¹). Interestingly, lengthened decay components of τ₂=0.192±0.009 μs and τ₃=7.6±0.6 s were observed upon DNA association, see FIGS. 38A and 38B. These are attributed to two distinct mechanisms: (1) the influence of the static electric field of DNA on T₂, which prolongs radical pair lifetime of ³[Cbz^{·+}-BP^{·-}]*, by delaying back-electron transfer to form ³[Cbz-BP]* (rate, ³k_{CRT} of 5.21×10⁶ s⁻¹) via electric field-spin coupling, consistent with its EPR spectral splitting upon DNA association (see, FIG. 24E). (2) Restricted molecular motion upon DNA binding, which slows non-radiative decay from ³[Cbz-BP]* to ¹I[Cbz-BP](rate, ³ k_{NR}=1.32×10⁵ s⁻¹), contributes to T₃. It is noted that a similar multi-component decay was observed even under ambient conditions, including freely diffusive oxygen-containing atmospheres (see, FIG. 42), confirming that DNA biosensing with this system is feasible without requiring inert or controlled environments.

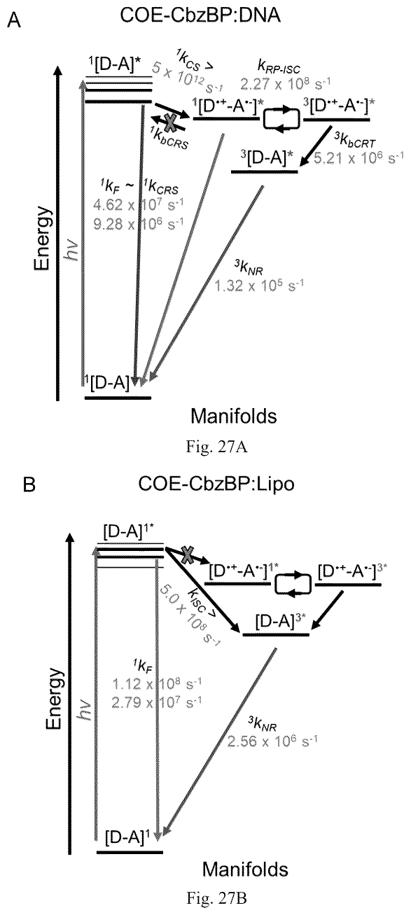

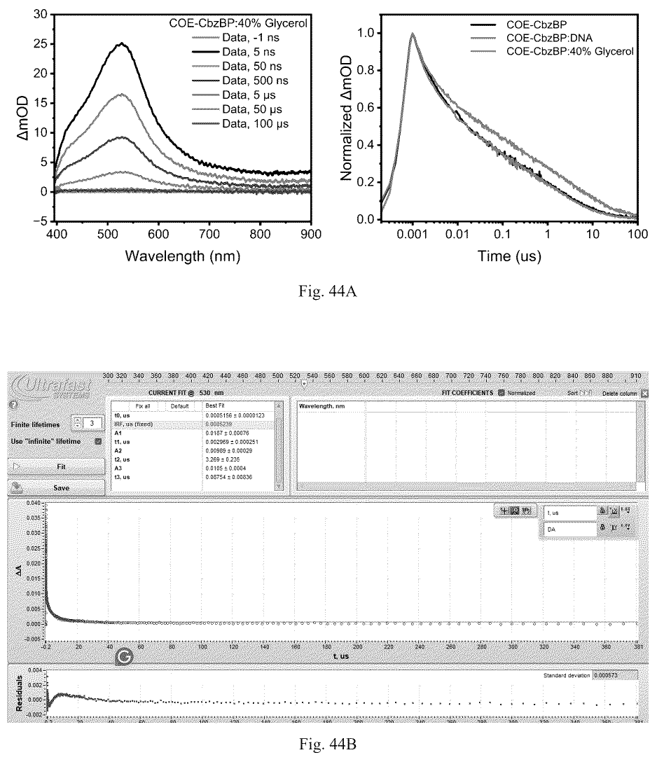

Conversely, when intercalated into lipid bilayers, a blue-shifted ESA (see FIG. 26E and FIG. 41) accompanied by a kinetic rise time in the nanosecond regime was followed by mono-exponential decay (τ=0.39±0.02 s; rate, ³K_{NR}=5.56×10⁶ s⁻¹), see FIG. 26F and FIG. 43. This photophysical profile is consistent with the formation of a molecular triplet ³[Cbz-BP]* due to near-planar zwitterionic geometry, similar to Michler's ketone, see FIG. 22F. As observed in the fs regime, glycerol-rich environments led to faster dynamics compared to glycerol-free conditions (FIGS. 44A and 44B and Table 7), while DNA association and MFE resulted in longer-lived ESA decays than in DNA-free conditions. These results align with the analysis that the spectral splitting observed via CW-EPR arises from electrostatic effects, rather than rheological factors, imparted by the static electric field of DNA on the SCRP photophysics of COE-CbzBP. See FIGS. 27A-27B for detailed energetics of the states and corresponding electronic rates of COE-CbzBP in DNA and liposomes.

### Example 8

The examples provided herein demonstrate a water-soluble, room-temperature photogenerated SCRP sensitive to magnetic-field effects and the electric field of freely diffusive biomolecules by combining molecular design principles from COEs with TADF-like chromophores. The observed qubit-like behavior arises from a minimized ΔE_{ST} of 0.12 eV, J_{RP} of 0.06 eV, and a thermodynamically favored ΔG_{CS}, made possible by introducing a partial break in π-conjugation at the N-C junction between the donor (carbazole) and acceptor (benzophenone). Spectroscopic and electrochemical analyses revealed that this partial break in conjugation promotes TICT geometry in freely diffusive environments. This conformation facilitates thermally accessible charge-separated radical pairs consistent with SCRP formation that is sensitive to the electric fields of DNA and to magnetic fields. Glycerol experiments further support that the electrostatic, rather than rheological, effects of DNA are responsible for perturbing the radical pair photophysics of COE-CbzBP. Supporting this assignment, EPR measurements in glycerol showed spectral broadening instead of the splitting observed with DNA, and TAS revealed faster dynamics, in contrast to the signal lengthening induced by the electric field of DNA. These findings highlight how geometry and local electrostatics govern the electronic and SCRP dynamics of COE-CbzBP, establishing TICT-enabled designs with small ΔE_{ST} as a viable route for creating optically addressable, water-compatible molecular probes with qubit-like properties. These insights open new opportunities for potential quantum biosensing in nucleic acid-rich environments and offer a versatile platform for tuning spin dynamics and excited-state properties in synthetic qubit-like scaffolds across biotic, abiotic, and broader QIS contexts.

1. A compound of formula (I): 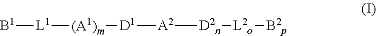
or a salt thereof, wherein
each of A¹ and A² is independently an electron acceptor;
each of D¹ and D² is independently an electron donor;
each of L¹ and L² is independently a π-conjugated linker;
each of B¹ and B² is independently an aryl substituted with one to four C₁-C₁₀ alkyl or C₁-C₁₀ alkoxy, wherein each of the alkyl or alkoxy is substituted with halogen, an ionic group, or a water-solubilizing group;
m is 0 or 1;
n is 0 or 1;
o is 0 or 1; and
p is 0 or 1;
provided that:
(i) m is 0 and n, o, and p are each 1;
(ii) n is 1 and m, o, and p are each 0;
(iii) each of m, n, o, and p are 0; or
(iv) each of m, n, o, and p are 1.

2. The compound of claim 1, wherein m is 0 and n, o, and p are each 1.

3. The compound of claim 1, wherein n is 1 and m, o, and p are each 0.

4. The compound of claim 1, wherein each of m, n, o, and p are 1.

5. The compound of claim 1, wherein each of A¹ and A² is independently of formula (II-E): 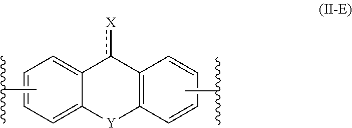
or a salt thereof, wherein
X is O or CR¹R²;
Y is absent, a bond, O, S, NH, Se, Te, C(O), or CR¹R²;
each R¹ is independently H, CN, C(O)OH, or C(O)—C₁-C₆ alkyl; and
each R² is independently H, CN, C(O)OH, or C(O)—C₁-C₆ alkyl.

6. The compound of claim 5, wherein each of A¹ and A² is independently a benzophenone.

7. The compound of claim 1, wherein each of D¹ and D² is independently of formula (III-A), 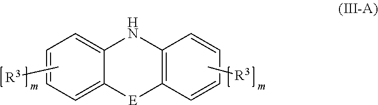
or a salt thereof, wherein
E is absent, a bond, O, S, Se, or Te;
each R³ is independently C₁-C₁₂ alkyl, C₁-C₆ alkoxy, 5- to 6-membered heteroaryl, or aryl, wherein aryl is optionally substituted with C₁-C₁₂ alkyl or C₁-C₆ alkoxy; and
each m is independently 0, 1, 2, 3, or 4.

8. The compound of claim 1, wherein each of D¹ and D² is independently a carbazole.

9. The compound of claim 8, wherein each of D¹ and D² is independently of formula: 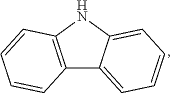 [SMILES: c1ccc2c(c1)[nH]c1ccccc12]
or a salt thereof.

10. The compound of claim 1, wherein each of L¹ and L² is an arylene-alkenylene.

11. The compound of claim 1, wherein each of B¹ and B² is independently a phenyl substituted with three C₁-C₁₀ alkoxy, and wherein each alkoxy is substituted with a water solubilizing group.

12. The compound of claim 11, wherein each water solubilizing group is a quaternary amine.

13. The compound of claim 1, wherein each of B¹ and B² is independently of the formula: 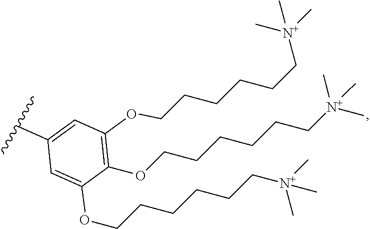 [SMILES: *c1cc(OCCCCCC[N+](C)(C)C)c(OCCCCCC[N+](C)(C)C)c(OCCCCCC[N+](C)(C)C)c1]
or a salt thereof.

14. The compound of claim 1, wherein the compound is of the formula: 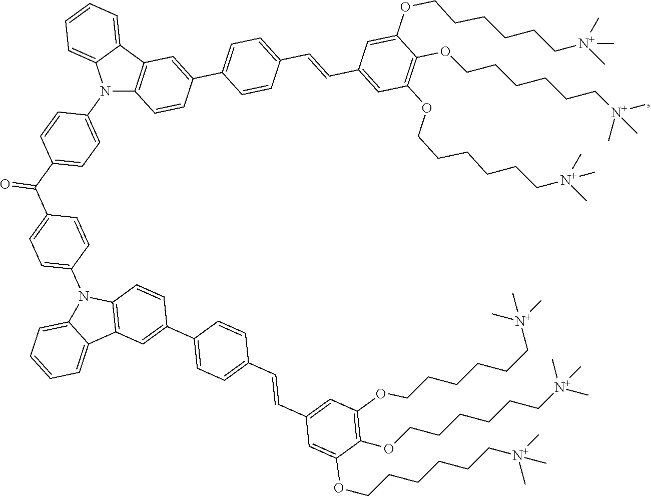 [SMILES: C[N+](C)(C)CCCCCCOc1cc(/C=C/c2ccc(-c3ccc4c(c3)c3ccccc3n4-c3ccc(C(=O)c4ccc(-n5c6ccccc6c6cc(-c7ccc(/C=C/c8cc(OCCCCCC[N+](C)(C)C)c(OCCCCCC[N+](C)(C)C)c(OCCCCCC[N+](C)(C)C)c8)cc7)ccc65)cc4)cc3)cc2)cc(OCCCCCC[N+](C)(C)C)c1OCCCCCC[N+](C)(C)C]
or a salt thereof.

15. The compound of claim 1, wherein the compound has a spin-correlated radical pair (SCRP).

16. A method of imaging a biological sample comprising:
(i) contacting a biological sample with the compound of claim 1;
(ii) exposing the biological sample and the compound to light of a first wavelength; and
(iii) detecting an emission of a second wavelength from the compound in the biological sample.

17. The method of claim 16, wherein the method is performed at about room temperature and under aqueous conditions.

18. The method of claim 17, wherein the biological sample comprises a nucleic acid, and wherein the compound interacts with the nucleic acid to form a twisted intramolecular charge-transfer (TICT) geometry.

19. The method of claim 16, wherein the method further comprises generating a spin-correlated radical pair (SCRP).

20. The method of claim 19, wherein the radical pair comprises an excited singlet state and an excited triplet state, and a singlet-triplet energy gap (ΔE_{ST}) of less than about 0.3 eV.
